(19) United States
(12) Patent Application Publication
(10) Pub. No.: US 2026/0055118 A1
(43) Pub. Date: Feb. 26, 2026

(54) VISIBLE LIGHT SENSITIVE PHOTOREMOVABLE PROTECTING GROUPS, PREPARATION PROCESS THEREOF, PHOTOACTIVATABLE CONJUGATES COMPRISING THEM AND USES THEREOF

(71) Applicant: HUN-REN TERMÉSZETTUDOMÁNYI KUTATÓKÖZPONT, Budapest (HU)

(72) Inventors: Márton BOJTÁR, Mány (HU); Alexandra EGYED, Várpalota (HU); Krisztina NÉMETH, Budapest (HU); Péter KELE, Budapest (HU)

(21) Appl. No.: 19/102,921

(22) PCT Filed: Aug. 8, 2023
(86) PCT No.: PCT/HU2023/050048

§ 371 Date: Feb. 11, 2025

(30) Foreign Application Priority Data
Aug. 11, 2022 (HU) P2200324
Oct. 10, 2022 (HU) P2200405

(51) Int. Cl. C07D 491/147; A61K 31/437; C07C 215/70; C07D 403/10; C07D 403/14; C07D 405/14; C07D 471/04

(52) U.S. Cl. CPC C07D 491/147; A61K 31/437; C07C 215/70; C07D 403/10; C07D 403/14; C07D 405/14; C07D 471/04

(57) ABSTRACT

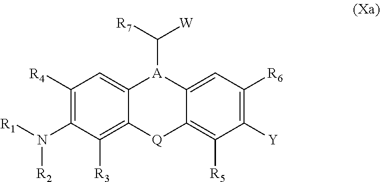

The present invention relates to visible light sensitive photoremovable protecting groups and their parent compounds X including a xanthene, xanthenium or related cores, represented by formula (Xa):(Xa) wherein the substituents are as defined in the description. The invention also relates to a process for the preparation of compounds of formula (Xa). Said compounds are parent compounds of visible light sensitive photoremovable protecting groups. They can be attached to any desired chemically or biologically active agent either via a covalent bond, or a linker. In the resulting conjugates the active agent is rendered inactive (or less active). Irradiation of such photoactivatable conjugates with visible light leads to the release of the agent with restored biological activity. Therefore, invention further relates to photoactivatable conjugates and pharmaceutical compositions containing them, which are suitable for use e.g. in photoactivated therapy.

### FIELD OF THE INVENTION

The present invention relates to visible light sensitive photoremovable protecting groups and their parent compounds X including a xanthene, xanthenium or related cores, which compounds can be attached to any desired chemically or biologically active agent either via a covalent bond, an oxycarbonyl linker or a self-immolative linker. In the resulting conjugates the active agent is rendered inactive (or less active). Irradiation of such photosensitive conjugates with visible light leads to the release of the agent with restored biological activity.

### BACKGROUND OF THE INVENTION

Photolabile or photoremovable protecting groups (PPGs) or photocages (PCs) are light-sensitive chemical moieties that are able to mask the chemical or biological function of substrates (active agents) through covalent linkages rendering them inert or less active¹⁻³. Upon irradiation with a suitable wavelength, the photoremovable protecting group is removed via bond cleavage (photolysis) and the activity of the active agent reinstates⁴⁻⁶. Examples for the use of photocaged compounds include chemical synthesis⁷, caged nucleotides⁸, neurotransmitters⁹, proteins⁵, pharmaceuticals¹⁰, ions¹¹, many other small molecules¹² and biomolecules¹³. Among other external stimuli, the spatial and temporal resolution of light is unsurpassed. Therefore, using photocaged compounds including transiently disabled biologically active agents, the manipulation of chemical/biological systems becomes possible with external light control¹⁴. The most popular photoremovable protecting groups used in biological studies are the o-nitrobenzyl, phenacyl, acridinyl, benzoinyl, coumarinyl groups¹⁵. Unfortunately, a significant limitation of these photocages is that they absorb mostly in the ultraviolet (UV) range, where the limited penetration of UV light into tissues largely restricts their use¹⁶. Furthermore, prolonged exposure of cells or tissues to intense UV light can lead to cellular damage or death. Advantages of visible light irradiation include diminished phototoxicity and deeper optical penetration¹⁴. Recent work featuring redshifted photoremovable protecting groups resulted in π-extended coumarin^{17,18}, BODIPY¹⁹⁻²¹ and cyanine frames²²⁻²⁴ as well as metal complexes^{25,26}. In the red region, modification of the BODIPY scaffold led to highly efficient visible to NIR light activatable cores^{21,27}, however, at the cost of poor water solubility and increased molecular weight. On the other hand, cyanines require the presence of molecular oxygen limiting their use in oxygen-defficient environments, such as solid tumors^{23,28}.

Following the success of photodynamic therapy (PDT), where light is combined with exogenously delivered sensitizers to trigger spatiotemporally controlled generation of reactive oxygen species, a novel concept, termed ‘photoactivated chemotherapy’ (PACT) has emerged²⁹. Compared to clinically approved PDT, PACT is based on the use of photoremovable protecting groups attached transiently to a cytotoxic S chemotherapeutic agent or an inhibitor³⁰. Upon light irradiation, the activity of the pharmaceutically active agent reinstates due to the liberation of the free agent. Since PACT does not rely on molecular oxygen, it would overcome the current limitations of PDT. Especially in malignant solid tumors, where the low oxygen concentration precludes the application of oxygen-dependent photosensitizers²⁹. Most PACT conjugates, however, are based on UV-absorbing photoremovable protecting groups¹⁰ that inherently limit their applications in deeper tissues¹⁶. Therefore, photoremovable protecting groups absorbing in the optical window of tissues (600-900 nm) that are also water soluble and release their cargo units oxygen-independently are urgently needed, especially for phototherapeutic applications such as PACT¹⁴.

An ideal photoremovable protecting group should meet several criteria¹⁵. Accordingly, the PPG and its is target conjugate: 1) should possess acceptable water-solubility within a given concentration range; 2) the target conjugate should be stable in the dark in aqueous media to prevent premature and uncontrolled release of the active agent; 3) to be suitable for clinical use, target conjugates with the photoremovable protecting groups should feature strong one-photon absorption above 550 nm with high molar absorption coefficient and efficient uncaging cross-sections (i.e., the photolysis rate should be fast to have sufficient photochemical quantum yields); 4) the target conjugate should be cell permeable; 5) the photolysis byproducts should be biologically inert and 6) synthesis of the target conjugates should allow late stage attachment of the cargo moiety, that is often a sensitive or hardly accessible drug, therefore it is of utmost importance to minimize any loss of it.

Development of novel PPGs is often inspired by the vast knowledge gained on the field of fluorescent dyes. Accordingly, the collection of visible-light sensitive photocages includes a wide variety of coumarins, BODIPYs, porphyrines or cyanines. The currently available palette, however, lacks one of the most popular scaffolds used exhaustively in fluorescent imaging schemes, i.e., xanthenium dyes such as rhodamines. The state of the art neither offers synthetic access to such compounds nor discloses the key structural details necessitated to their stability.

U.S. Pat. No. 9,957,498 discloses a set of photoremovable protecting groups based on a BODIPY chromophore developed by Winter et al.,²¹ for the release of organic molecules, however, these compounds have limited photolysis rates and water solubility.

US 2002/0016472 discloses protecting groups derived either from a halogenated coumarin or a xanthene group. The protecting groups can be removed by irradiating the group with light, by using e.g., flash photholysis with ultraviolet radiation or pulsed infrared radiation. We note that although xanthene-based protecting groups are envisaged in this document, the only compound synthesized is 9-chloromethylxanthene. The concept of photoremovable protecting groups was not demonstrated with this class of compound since no examples were given beside the sole chloromethyl derivative and the chloride ion can not be considered as leaving group in this respect. Importantly, the described synthetic route to access the scaffold proved to be unsuitable for 1) attachment of a cargo moiety other than a simple chloride 2) rhodol- and rhodamine-based derivatives.

The disclosed PPG-s are not suitable for one-photon absorption above 550 nm (with efficient uncaging cross sections), the longer wavelength (pulsed infrared) activation was based on two-photon absorption that is certainly a useful methodology for uncaging small quantities but unsuitable for phototherapeutic applications due to the ultra-small size of the irradiated focal point (i.e., picoliters in volume).

WO 2021/071876 discloses photolytic compounds and triplet-triplet annihilation mediated photolysis. In this document, the disadvantages of using short wavelength light for photolysis are detailed, and it is mentioned that attempts to use far red light and near infrared light have failed. As a response to these drawbacks, the above document discloses a method of conducting photolysis mediated by triplet-triplet annihilation, which can be used with a pair of a PPG and a photosensitizer, a concept significantly different from the present invention (i.e., multiple components, nanoparticle requirements, etc.). Among the PPGs, anthracene derivatives are mentioned. Said anthracene derivatives are not suitable for phototherapeutic applications by themselves (as PPG moieties in target conjugates) due to their UV-absorbtion (no absorption in the visible range) and poor water solubility. As the inventors in said document presented, their anthracene (and other) derivatives are in fact not uncaging by themselves using visible light irradiation (see Table 12 for an example) and in the biology experiments only the nanoparticles were utilized.

Kln et al.² mentions, among others, protecting groups based on xanthene and pyronine moieties. It is stated that (6-hydroxy-3-oxo-3H-xanthen-9-yl)methyl derivatives release diethyl phosphate or carboxylic acid upon irradiation with visible light, however, in their original report,³¹ they could only isolate a fluorescein derivative in the form of a DDQ (2,3-dichloro-5,6-dicyano-1,4-benzoquinone) complex. Although pyronine analogues are suggested to be used in the field of photoremovable protecting groups, in their later article,³² they admitted that attempts to install a saturated oxymethyl linker onto the C9 position, as well as many alternative synthetic procedures failed, so they decided to employ the said BODIPY compounds as alternative chromophores. Importantly, although Klán et al. mentioned the possibility of xanthenium PPGs, their lack of success clearly indicates that accessing such compounds is not trivial and further modifications are required.

Kitamura et al.³³ describe a rodamine based nitric oxide donor. The disclosed molecules contain a rhodamine moiety, but they cannot be regarded as protecting groups (the document does not even mention photoremovable protecting groups) since the released molecule (nitric oxide) cannot be replaced with any desired active agent.

To conclude, there are some examples in state of the art suggesting the use of xanthene-based groups, however, none of them offers any feasible photoremovable protecting group with xanthete-derived frames, and rather reports unsuccessful or cumbersome synthesis to access the envisioned structures.

Therefore, our aim was to provide photoremovable protecting groups with xanthenium scaffolds, which meet the above-mentioned general criteria for PPGs.

### SUMMARY OF THE INVENTION

We have found that compounds X (represented by formulas (Xa) or (Xd)) can be readily synthesized and isolated, and are suitable precursor compounds of photoremovable protecting groups. The photoactivatable conjugates that can be prepared from compound X have significant advantages over conjugates containing known photoremovable protecting groups. The advantages of the photoactivatable conjugates are attributed to the novel photoremovable protecting groups derived from compound X. These advantages are (1) high solubility in aqueous media (2) one-photon absorbance between 550-700 nm (3) high photolysis rates and consequently high one-photon uncaging cross sections (4) cell permeability (5) simple synthesis enabling late stage attachment of the active agent. As will become evident to those skilled in the art, utilizing the derivatives of compounds X as photoremovable protecting groups attached to various active agents are optimal tools for manipulating biological systems with light as well as treating localized diseases.

Therefore, the present invention relates to compounds represented by formula (Xa),

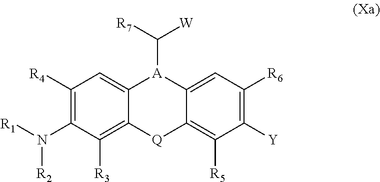

wherein
A is either C⁺ or CH,
   when A is C⁺, the compound comprises a counter anion, which is preferably a pharmaceutically acceptable anion;
R₁ and R₂ are each selected independently from hydrogen, C₁-C₆ alkyl, C₃-C₆ cycloalkyl, C₂-C₆ alkenyl, C₆-C₁₀ aryl; or
R₁ and R₂, together with the nitrogen atom, to which they are attached, form a 3-10-membered heterocycloalkyl ring containing said N atom, and optionally containing a further heteroatom selected from N, O and S;
Y is selected from NR₈R₉ or OR₁₀, wherein
   R₈ and R₉ are each independently selected from hydrogen, C₁-C₆ alkyl, C₃-C₆ cycloalkyl, C₂-C₆ alkenyl and C₆-C₁₀ aryl; or
   R₈ and R₉, together with the nitrogen atom, to which they are attached, form a 3-10-membered heterocycloalkyl ring containing said N atom, and optionally containing a further heteroatom selected from N, O and S;
   R₁₀ is selected from hydrogen and OH-protecting group;
R₃, R₄, R₅, Ra are each selected independently from H, halogen, C₁-C₆ alkyl, C₃-C₆ cycloalkyl or C₃-C₆ cycloalkenyl, or
R₃ and R₂, R₄ and R₁, R₅ and R₉, and/or R₆ and R₈ together with the intervening atoms, may form a 5-7-membered non aromatic, saturated or unsaturated heterocycle containing a N atom, and optionally containing a further heteroatom selected from N, O and S,
R₇ is C₁-C₆ alkyl, C₂-C₆ alkenyl, C₃-C₆ cycloalkyl, C₆-C₁₀ aryl or (C₆-C₁₀ aryl)(C₁-C₆ alkyl);
Q is selected from NH, NR₁₁, O, C(R₁₁)₂, Si(R₁₁)₂, P=O(Ph) or P=O(OPh) or P═O(O⁻), wherein
   each R₁₁ is independently selected from C₁-C₆ alkyl, C₃-C₆ cycloalkyl, C₂-C₆ alkenyl, C₆-C₁₀ aryl and (C₆-C₁₀ aryl)(C₁-C₆ alkyl);
W is either
   OH,
   halogen, or a
   linker precursor moiety,
wherein
   in the meaning of R₁ to R₁₁ each instance of alkyl and alkenyl is optionally substituted with one or more substituent(s) independently selected from the group of halogen, hydroxyl and CF₃, in the meaning of R₁ to R₁₁ each instance of cycloalkyl is optionally substituted with one or more substituent(s) independently selected from the group of C₁-C₄ alkyl, halogen, hydroxyl and CF₃, the 3-10-membered heterocycloalkyl formed by NR₁R₂ or NRsR₉ is optionally substituted with 1-3 substituent(s) independently selected from the group of C₁-C₄ alkyl, halogen, hydroxyl and CF₃, the 5-6-membered non aromatic, saturated or unsaturated heterocycle formed by R₃ and R₂, R₄ and R₁, R₅ and R₉, and/or R₆ and R₈ together with the intervening atoms is optionally substituted with one or more substituent(s) independently selected from the group of C₁-C₄ alkyl, halogen, hydroxyl and CF₃,
   in the meaning of R₁, R₂, R₇-R₉ and R₁₁ each instance of aryl is optionally substituted with one or more substituent(s) independently selected from the group of C₁-C₄ alkyl, halogen and CF₃,
in any protonation states thereof, or salts thereof.

More specifically, in case A represents C⁺ and Y represents OH, the deprotonated forms of the compounds are also included herein. Accordingly, the invention also relates to a compound represented by formula (Xd)

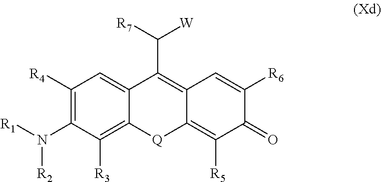

wherein Q, W and R₁ to R₇ are as defined above.

The invention also relates to a process for the preparation of a compound of formula (Xa), including:
a) reacting a compound of formula (I):

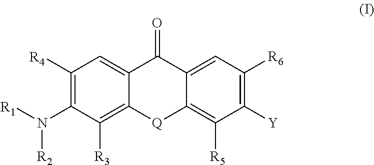

with a Grignard reagent of formula R₇—CH₂MgHal, wherein Hal is halogen, or with a compound of formula R₇CH₂Li, and converting the resulting adduct to an olefin derivative using first a proton source and then subsequent deprotonation using a strong base, to obtain a compound of formula (II):

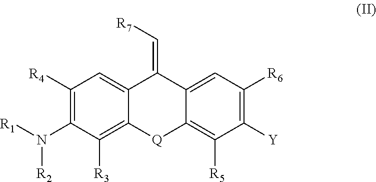

subjecting the compound of formula (II) to hydroboration-oxidation reaction for the anti-Markovnikov addition of a hydroxyl group and a hydrogen to the olefin bond (e.g. using a borane reagent then hydrogen peroxide) to obtain a compound of formula (Xa) wherein A=CH and W═OH:

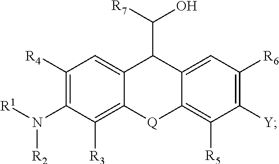

or
b) reacting a compound of formula (I):

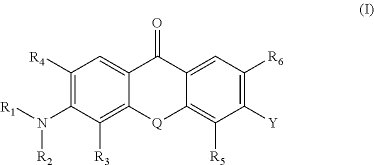

with a deprotonated vinyl ether derivative, e.g. which is performed by treatment of a vinyl ether of formula R₁₄CH—OAlk with a strong base, wherein R₁₄ represents e.g. CH₂═ or CH₃—(CH₂)ₙ—CH═ wherein n is an integer from 0 to 5, wherein said R₁₄ can be considered as a precursor group of R₇ and Alk refers to an alkyl group,
and hydrolysing the resulting vinyl ether appended compound to obtain a compound of formula (III):

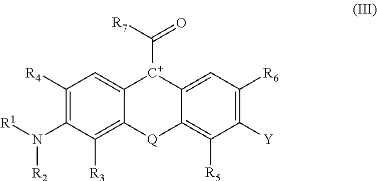

reducing the compound of formula (III), to obtain a compound of formula (Xa) wherein A=CH and W═OH:

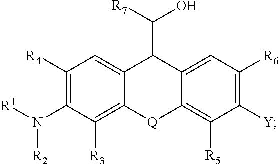

or
c) reacting a compound of formula (I):

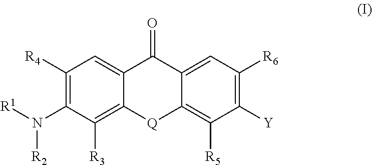

with a Grignard reagent of formula R₇—CH₂MgHal, wherein Hal is halogen, or with a compound of formula R₇CH₂Li, and converting the resulting adduct to an olefin derivative using first a proton source and then subsequent deprotonation using a strong base, to obtain a compound of formula (II):

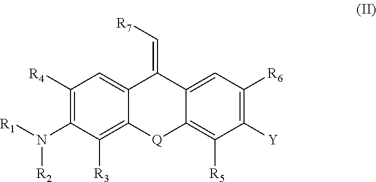

reacting the compound of formula (II) with N-halogenosuccinimide in the presence of water for the addition of HO-Hal to the olefin bond, then eliminating a hydroxide ion, to obtain a compound of formula (Xa) wherein A=C⁺ and the compound comprises a counter anion, designated as “Anion⁻”, and W=halogen:

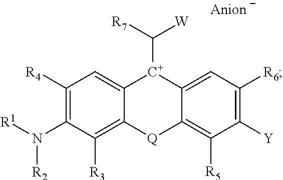

and optionally
d) converting the compounds of formula (Xa) wherein W═OH to compounds of formula (Xa) wherein W=oxycarbonyl linker precursor moiety using carbonic acid-derived activation reagents (such as DSC or 4-nitrophenyl chloroformate)
and optionally
e) converting the compounds of formula (Xa) wherein W=oxycarbonyl linker precursor moiety to compounds of formula (Xa) wherein W=self-immolative linker precursor moiety by reaction with a self-immolative linker precursor compound (e.g. with either N-Boc-N,N′-dimethylethylenediamine in the presence of a base, or 4-hydroxymethylaniline in the presence of a base and 1-hydroxybenzotriazole),
and optionally
f) oxidizing the compounds of formula (Xa) wherein A=CH using chloranil and subsequent anion replacement to compounds of formula (Xa) wherein A=C⁺.

The invention also relates to the use of the compound represented by formula (Xa) or (Xd) as a precursor for a photoremovable protecting group.

Furthermore, the invention relates to photoactivatable conjugates comprising a photoremovable protecting group derived from a compound represented by formula (Xa) or (Xd), or salt thereof.

The photoremovable protecting group derived from a compound represented by formula (Xa) or (Xd) is represented by formulas (Xa′) or (Xd′):

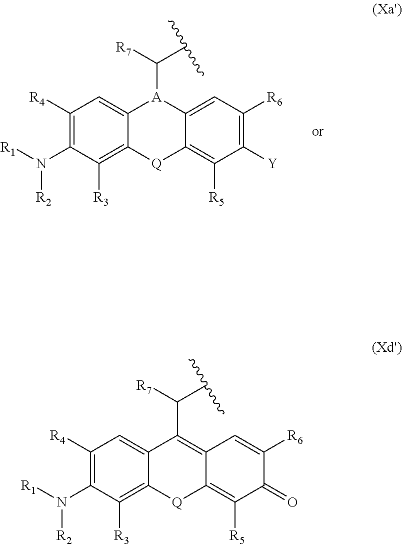

wherein A is C⁺ and the compound comprises a counter anion,
Y, Q, and R₁ to R₇ are as defined above, and
the wavy line ( [symbol] ) represents the attachment point to the rest of the molecule.

The invention also relates to a pharmaceutical composition comprising the photoactivatable conjugate or a pharmaceutically acceptable salt thereof according to the invention, together with one or more pharmaceutically acceptable excipient.

The invention also relates to the photoactivatable conjugate or a pharmaceutically acceptable salt thereof for use in photoactivated therapy.

The invention furthermore relates to a method for photoreleasing an active agent Z from a photoactivatable conjugate of a formula X′-L-Z′, comprising irradiating a biological sample selected from cell cultures or xenograft tumor models containing X′-L-Z′ with a wavelength >550 nm so as to release the active agent Z, wherein X′ is a photoremovable protecting group derived from a compound represented by formula (Xa) or (Xd), L is a covalent bond or a linker, and Z′ is a moiety derived from an active agent.

More particular embodiments of the invention are set out in the dependent claims and in the detailed description.

### Definitions Used in the Description

In the description, the following definitions are used:
chemical moiety: a part of a molecule with a distinct functionality, such as a chromophore, a protecting group, a self-immolative linker precursor group or a photoremovable protecting group. A chemical moiety is usually derived from a distinct chemical entity or a precursor molecule.
chemical derivatization: when a specific functional group in a given molecule is converted to alter the function of the given molecule, it can be considered as chemical derivatization. For example, when two distinct molecules (A and B) are connected via a chemical linkage, the resulting conjugate (A-B) can be considered as a distinct molecule with two chemical moieties, a moiety (A′) that is the result of chemical derivatization of A by B and a moiety (B′) that is the result of chemical derivatization of B by A.
active agent (Z): a chemically or biologically active small molecule or biomolecule that can be rendered inert (or less active) by disabling its key functional group by covalent modification. Such molecule could be, but not restricted to for example, an oligopeptide, protein, enzyme, nucleotide, nucleic acid, carbohydrate, lipid, neurotransmitter, catalyst, fragrance, pharmaceutically active ingredient or drug, anticancer agent, chemotherapeutic agent, small molecule inhibitor, fluorescent dye or fluorogenic dye. When the active agent is attached to an other molecule, such as compound X (via chemical derivatization), it is considered as the cargo unit (Z′) of the resulting conjugate (also known in the literature as payload). In other words, the chemical moiety derived from the active agent is the cargo unit.
compound X: these molecules are the necessary precursors for attachment of the photoremovable protecting group on a desired active agent. They are represented by formulas (Xa) and (Xd). Compound X therefore can be attached or coupled to any desired active agent resulting in the target conjugates. When compound X is attached to the active agent, it is no longer a distinct molecule but a chemical moiety that is considered as the photoremovable protecting group or photocage (X′). Therefore, in the description, X is also referred to as the parent compound of the X′ photoremovable protecting group. The X′ photoremovable protecting groups are represented by formulas (Xa′) and (Xd).
target conjugate or photoactivatable conjugate: attachment of compound X to an active agent results in the target conjugate, which is a distinct molecule. These molecules contain three chemical moieties that are the derivatives of the active agent Z (Z′) and compound X (X′) and a linker (L) between the chemical moieties. The target conjugates therefore consist of a photoremovable protecting group X′, the cargo unit Z′ and a linker. These are the actual photoactivatable conjugates.
connecting chemical moiety or linker (1): the chemical moiety or covalent bond between the cargo unit and the photoremovable protecting group.
oxycarbonyl linker: refers to an —O—(C═O)— or —O—(C═S)— moiety that is often required for the attachment of the cargo unit to a photoremovable protecting group.
oxycarbonyl linker precursor moiety: a carbonic acid-derived group that readily reacts with —OH and —NH₂ groups on the active agent, and forms an oxycarbonyl linker between the resulting photoremovable protecting group and the cargo unit.
self-immolative linker: refers to a covalent chemical assembly, commonly used for therapeutic or bioanalytical aims, wherein a primary reaction initiates internal molecular rearrangements leading to the release of a substrate (e.g., a drug or a probe, in this case, the active agent).³⁴
self-immolative linker precursor moiety: a chemical group that can be converted to a self-immolative linker by the attachment of the cargo unit.

### Abbreviations

Bn benzyl
Boc tert-butoxycarbonyl
DCC N,N′-dicyclohexylcarbodiimide
DCM dichloromethane
DMAP 4-dimethylaminopyridine
DMF N,N-dimethylformamide
DMSO dimethyl sulfoxide
DSC N,N′-disuccinimidyl carbonate
EC50 half maximal effective concentration
equiv. equivalent
Et ethyl-
EtOH ethanol
EtOAc ethyl-acetate
FBS Fetal Bovine Serum
GSH glutathione
HEPES N-(2-hydroxyethyl)piperazine-N′-(2-ethanesulfonic acid)
HOBt 1-hydroxybenzotriazole
HPLC high-performance liquid chromatography
HRMS high-resolution mass spectrometry
KOtBu potassium tert-butoxide
IC50 half maximal inhibitory concentration
LC liquid chromatography
LED light-emitting diode
M mol/dm³
Me methyl-
MeCN acetonitrile
MeOH methanol
MOM methoxymethyl-(protecting group)
MS mass spectrometry
MTT 3-(4,5-dimethylthiazol-2-yl)-2,5-diphenyltetrazolium bromide
NBS N-bromosuccinimide
NMR nuclear magnetic resonance
on overnight
PBS phosphate-buffered saline
Ph phenyl-
Py pyridine
RT room temperature
TEA triethylamine
THF tetrahydrofuran

### BRIEF DESCRIPTION OF THE DRAWINGS

FIG. 1 shows a scheme of the concept of photoremovable protecting groups. First, compound X, the precursor of the photoremovable protecting group is attached to the active agent (i.e., a chemotherapeutic drug) via the connecting chemical moiety, resulting in the photoactivatable target conjugate after the appropriate chemical steps. The active agent is disabled in the conjugate form due to the chemical modification of a key functional group necessary for its biological activity. Upon irradiation with visible light, the active agent is released and its activity reinstates.

FIG. 2a shows the general structure and examples of compounds X described herein (*) with additional analogous exemplary structures wherein A=CH and W═OH

FIG. 2b shows examples of compounds X described herein (*) with additional exemplary structures wherein A=CH and W═OH or halogen precursor moiety

FIG. 2c shows examples of compounds X described herein (*) with additional exemplary structures wherein W=oxycarbonyl linker precursor moiety or self-immolative linker precursor moiety

FIGS. 3a, 3b and 3c show examples of target conjugates that represent the variability of the photoremovable protecting groups, connecting chemical moieties and cargo units that can be prepared from compounds X.

FIG. 4 shows the structure of two photoactivatable conjugates X1′-SN38 and X2′-SN38 consisting of a photoremovable protecting group, a self-immolative linker and a cargo unit derived from a pharmaceutical agent, namely the topoisomerase I inhibitor 7-ethyl-10-hydroxycamptothecin, also known as SN38. Also shown is the mechanism of release and the byproducts of photolysis. The photoactivatable conjugate has low potency towards the target of the drug, topoisomerase 1. Upon irradiation with red light using an LED light source, the active agent is released via light-induced bond cleavage and subsequent self-immolation reaction. The high potency of the active agent SN38 and its activity reinstates resulting in cell death.

FIG. 5 shows the structure and absorption spectra of selected target conjugates.

FIG. 6a shows uncaging experiments of compound 39 as followed by an HPLC-UV-MS instrument. Top: structure of the target conjugate and the scheme of the uncaging process. Left: partial chromatograms of the irradiated samples. A sample of 1 ml of the target conjugate with a concentration of 0.1 mM in water-MeCN 9:1 was irradiated for various time periods using an orange LED light source (^{˜}605 nm). The release of phenylacetic acid (C) was monitored at 195 nm, the disappearance of the starting material (A) was monitored at 570 nm. The photoproduct of the photoremovable protecting group (B) is also apparent. Right: partial chromatograms of the non-irradiated dark samples under similar experimental conditions.

FIG. 6b shows uncaging experiments of compound 41 as followed by an HPLC-UV-MS instrument. Top: structure of the target conjugate and the scheme of the uncaging process. Left: partial chromatograms of the irradiated samples. A sample of 1 ml of the target conjugate with a concentration of 0.1 mM in water-MeCN 9:1 was irradiated for various time periods using a green LED light source (^{˜}550 nm). The release of phenylacetic acid (C) was monitored at 195 nm, the disappearance of the starting material (A) was monitored at 530 nm. The photoproduct of the photoremovable protecting group (B) is also apparent. Right: partial chromatograms of the non-irradiated dark samples under similar experimental conditions.

FIG. 6c shows uncaging experiments of compound 43 as followed by an HPLC-UV-MS instrument. Top: structure of the target conjugate and the scheme of the uncaging process. Left: partial chromatograms of the irradiated samples. A sample of 1 ml of the target conjugate with a concentration of 0.1 mM in water-MeCN 9:1 was irradiated for various time periods using a red LED light source (^{˜}658 nm). The release of phenylacetic acid (C) was monitored at 195 nm, the disappearance of the starting material (A) was monitored at 640 nm. The photoproduct of the photoremovable protecting group (B) is also apparent. Right: partial chromatograms of the non-irradiated dark samples under similar experimental conditions.

FIG. 6d shows uncaging experiments of compound 45 as followed by an HPLC-UV-MS instrument. Top: structure of the target conjugate and the scheme of the uncaging process. Left: partial chromatograms of the irradiated samples. A sample of 1 ml of the target conjugate with a concentration of 0.1 mM in water-MeCN 9:1 was irradiated for various time periods using a red LED light source (^{˜}658 nm). The release of phenylacetic acid (C) was monitored at 195 nm, the disappearance of the starting material (A) was monitored at 660 nm. The photoproduct of the photoremovable protecting group (B) is also apparent. Right: partial chromatograms of the non-irradiated dark samples under similar experimental conditions.

FIG. 6e shows uncaging experiments of compound 49 as followed by an HPLC-UV-MS instrument. Top: structure of the target conjugate and the scheme of the uncaging process. Left: partial chromatograms of the irradiated samples. A sample of 1 ml of the target conjugate with a concentration of 0.1 mM in water-MeCN 9:1 was irradiated for various time periods using a red LED light source (^{˜}658 nm). The release of N-phenylpiperazine (C) was monitored at 254 nm, the disappearance of the starting material (A) was monitored at 640 nm. The photoproduct of the photoremovable protecting group (B) is also apparent. Right: partial chromatograms of the non-irradiated dark samples under similar experimental conditions.

FIG. 6f shows uncaging experiments of compound 51 as followed by an HPLC-UV-MS instrument. Top: structure of the target conjugate and the scheme of the uncaging process. Left: partial chromatograms of the irradiated samples. A sample of 1 ml of the target conjugate with a concentration of 0.1 mM in water-MeCN 9:1 was irradiated for various time periods using a red LED light source (^{˜}658 nm, 50% power). The release of phenol (C) was monitored at 195 nm, the disappearance of the starting material (A) was monitored at 640 nm. The photoproduct of the photoremovable protecting group (B) is also apparent. Right: partial chromatograms of the non-irradiated dark samples under similar experimental conditions. Note that in this case, the dark stability of the target conjugate is reduced.

FIG. 6g shows uncaging experiments of compound X1′-SN38 as followed by an HPLC-UV-MS instrument. Top: structure of the target conjugate and the scheme of the uncaging process. Left: partial chromatograms of the irradiated samples. A sample of 1 ml of the target conjugate with a concentration of 0.1 mM in water-MeCN 9:1 was irradiated for various time periods using an orange LED light source (^{˜}605 nm). The release of the SN38-adduct (C) and SN38 (D) was monitored at 369 nm, the disappearance of the starting material (A) was monitored at 570 nm. The photoproduct of the photoremovable protecting group (B) is also apparent. Right: partial chromatograms of the non-irradiated dark samples under similar experimental conditions.

FIG. 6h shows uncaging experiments of compound X2′-SN38 as followed by an HPLC-UV-MS instrument. Top: structure of the target conjugate and the scheme of the uncaging process. Left: partial chromatograms of the irradiated samples. A sample of 1 ml of the target conjugate with a concentration of 0.1 mM in water-MeCN 9:1 was irradiated for various time periods using a red LED light source (^{˜}658 nm). The release of the SN38-adduct (C) and SN38 (D) was monitored at 369 nm, the disappearance of the starting material (A) was monitored at 640 nm. The photoproduct of the photoremovable protecting group (B) is also apparent. Right: partial chromatograms of the non-irradiated dark samples under similar experimental conditions.

FIG. 7a shows the structure of the target conjugate X2′-SN38 (top) and confocal microscopy images showing the localization of the particular target conjugate in SK-OV-3 cells. The cells were incubated with 1 M compound together with 50 nM MitoTracker Red or LysoTracker Red. The separate channels are shown as well as a trace that provides information on colocalization. From these images, it is evident that compound X2′-SN38 localizes in the lysosomes.

FIG. 7b shows structure of the target conjugate X2′-SN38 (top) and confocal microscopy images showing the high targeting ability of light. The area marked with a square was irradiated using a 638 nm laser light source. The decrease of fluorescent signal solely at the area of irradiation demonstrates the high spatiotemporal control achievable by light. In each case, SK-OV-3 cells were treated with 1 μM of the compound.

FIG. 8a shows the structure of the target conjugate X2′-SN38 (top) and cell viabilities from MTT assay. Cells were treated with different concentrations of the particular target conjugate and illuminated for various time periods. The viabilities are relative to vehicle (DMSO) treatment.

FIG. 8b shows the structure of the target conjugate X1′-SN38 (top) and concentration-dependent viabilities for the EC50 value determination of the particular target conjugate. Cells were irradiated for 5 min by orange light (605 nm). The viabilities are relative to vehicle (DMSO) treatment.

FIG. 8c shows the structure of the target conjugate X2′-SN38 (top) and concentration-dependent viabilities for the EC50 value determination of the particular target conjugate. Cells were irradiated for 60 s by red light (658 nm). The viabilities are relative to vehicle (DMSO) treatment.

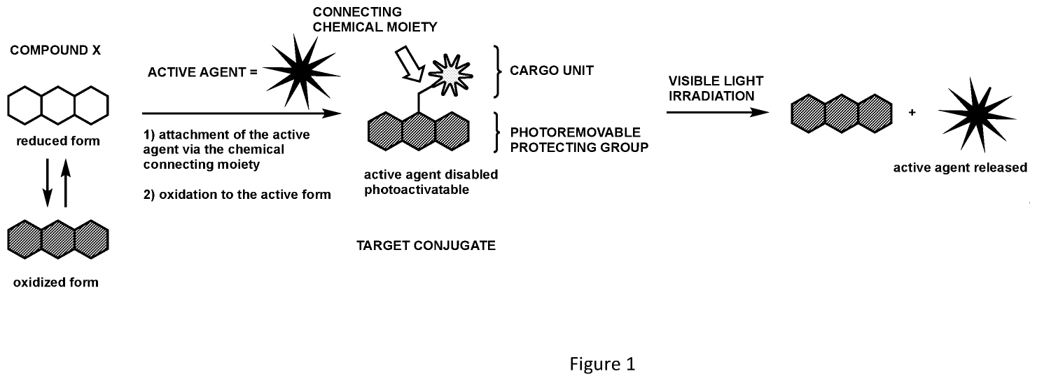

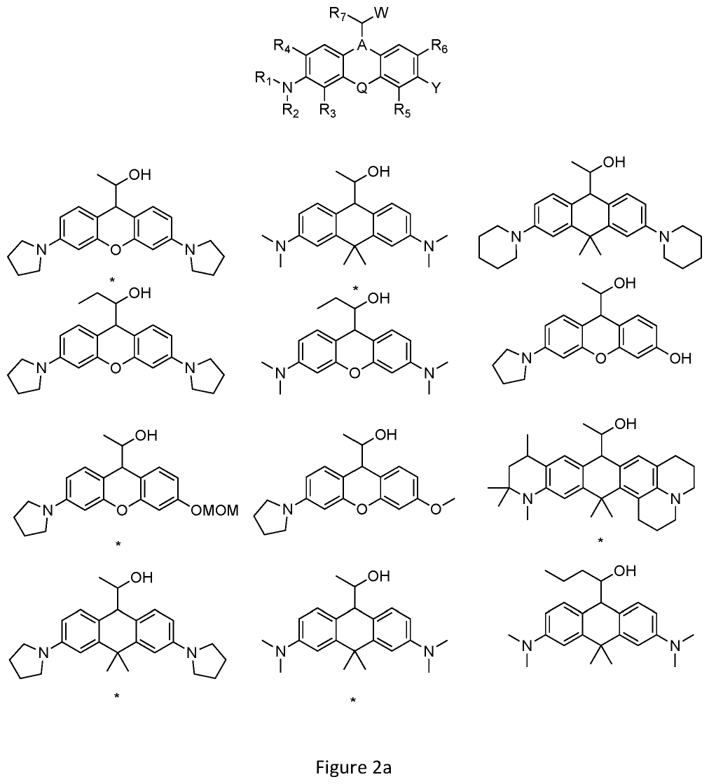

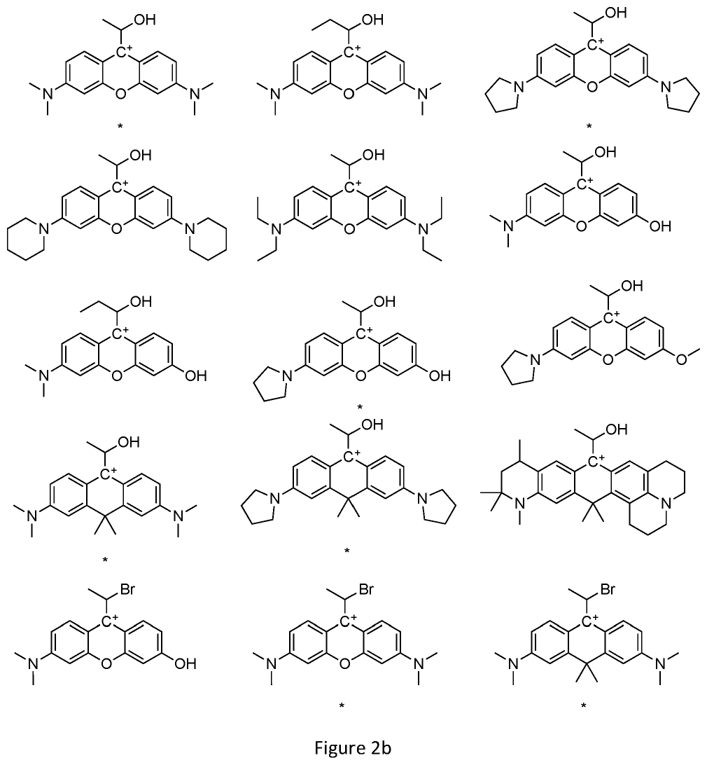

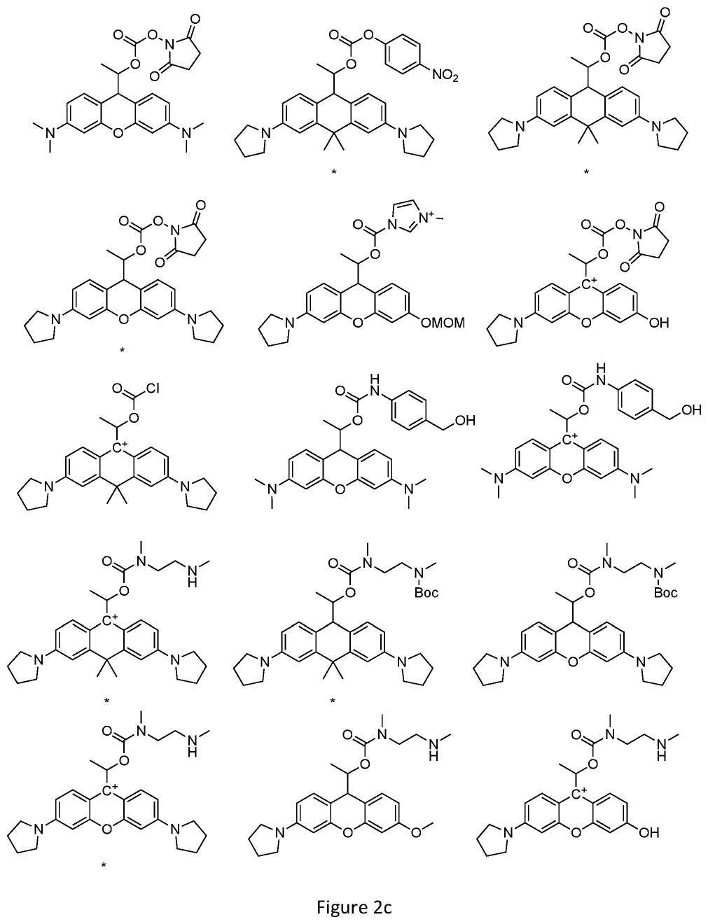

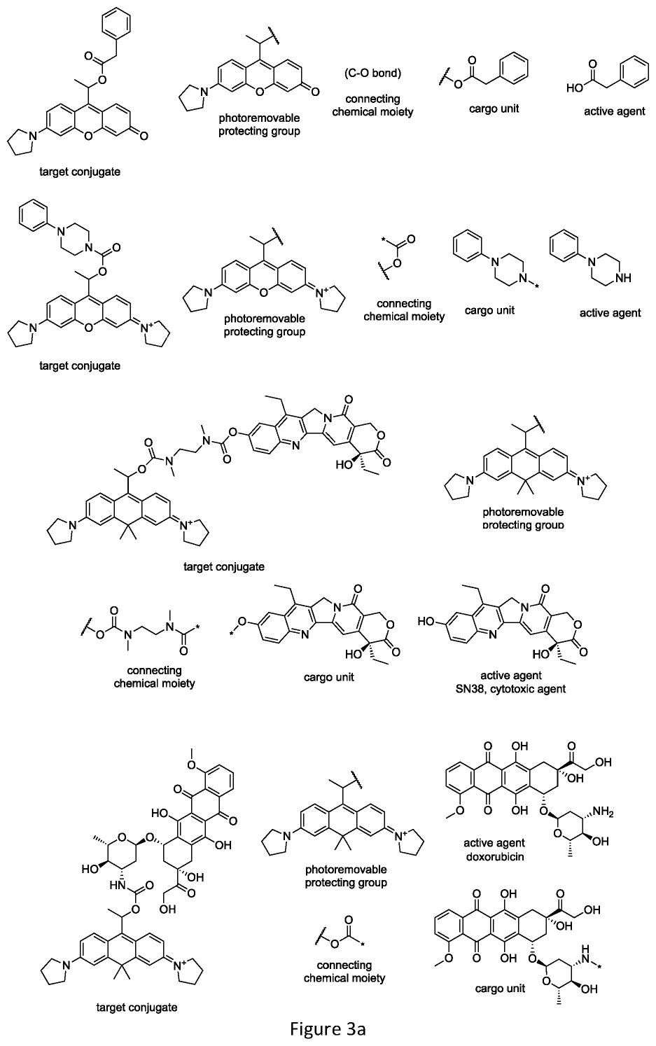

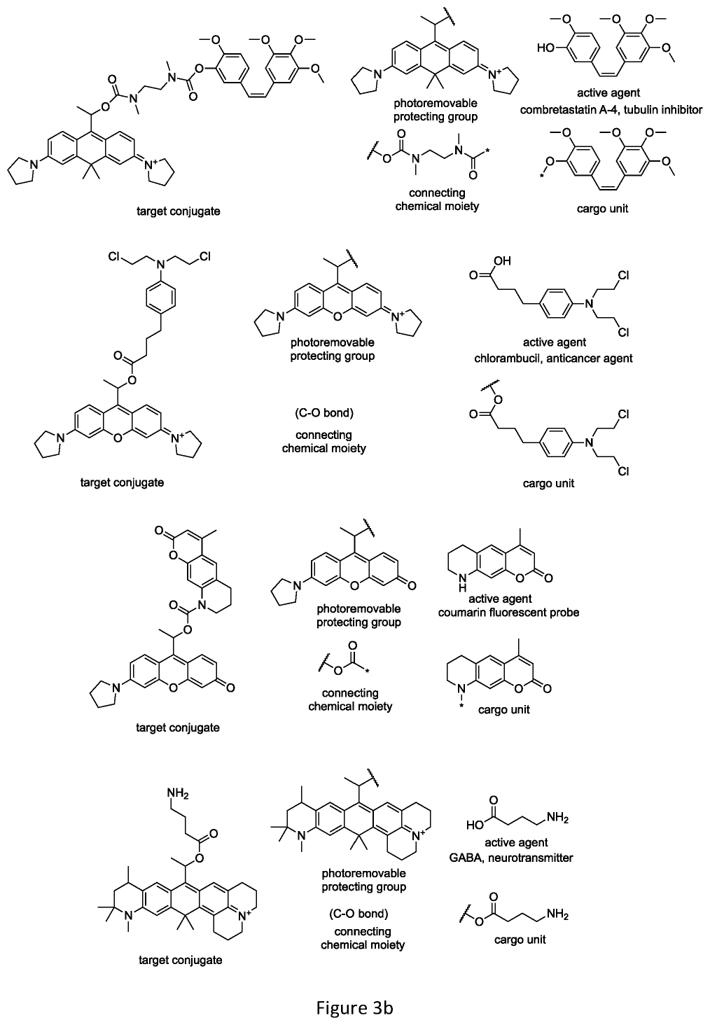

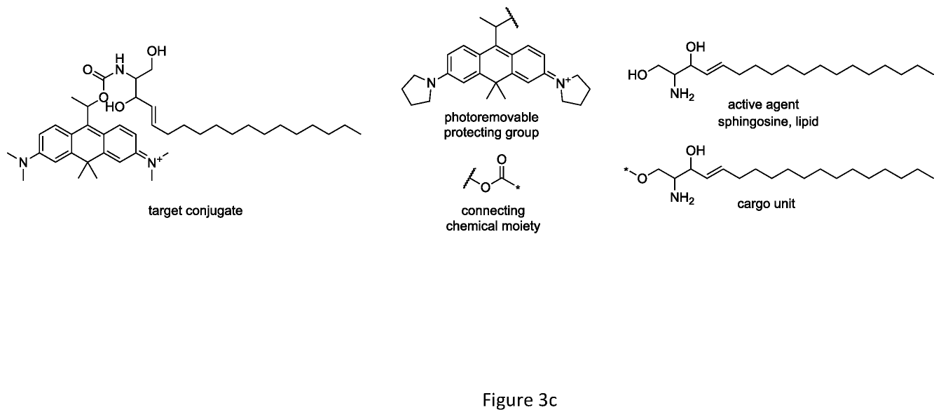

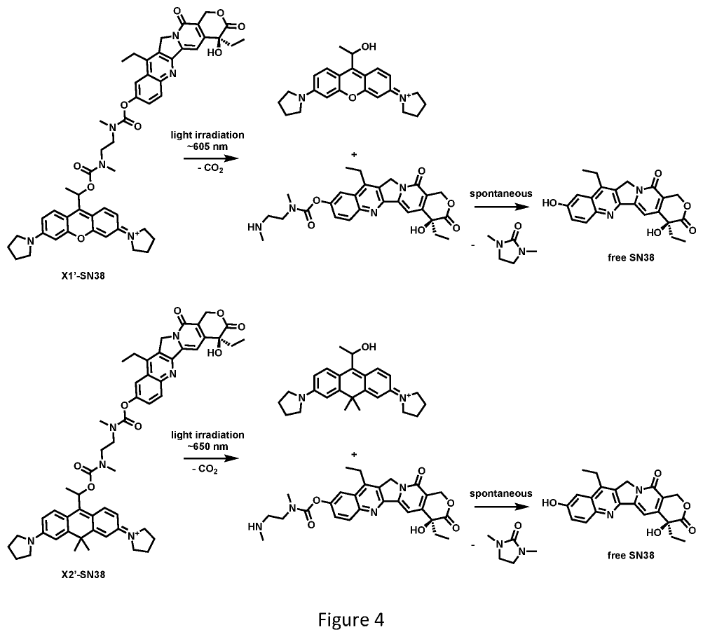

### DETAILED DESCRIPTION OF THE INVENTION

### 1. Structure of the Compounds X

The present invention relates to xanthene or xanthenium-derived compounds (abbreviated as compounds X) that can be attached to any desired active agent resulting in target conjugates or photoactivatable conjugates. Upon attachment of the active agent, the chemical moiety derived from compound X is called a photoremovable protecting group (abbreviated as moiety X′) and the chemical moiety derived from the active agent becomes the cargo unit (abbreviated as moiety Z′). In other words, the target conjugates are composed of the photoremovable protecting group (X′) the cargo unit (Z′) and a connecting chemical moiety or linker (L) necessary to form the specific chemical connection between compound X and the active agent, i.e., the target conjugates can be described as X′-L-Z′. Importantly, when the active agent is in its photoactivatable (or photocaged) form, its chemical and biological activity is disabled or reduced. These target conjugates absorb in the upper visible range (>550 nm) and release the active agent upon irradiation.

### 1.1 General Structure

The compound X is a substituted xanthenium or xanthene-based (or related) compound, represented by the following formula:

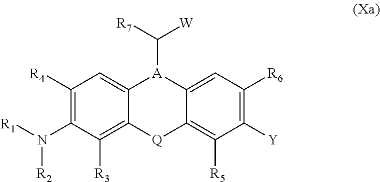

In formula (Xa), A is either C⁺ or CH, and when A is C⁺, the compound comprises a counter anion.

Importantly, the structures wherein A=C⁺ and CH can be interconverted via oxidation or reduction using standard methods known for those skilled in the art:

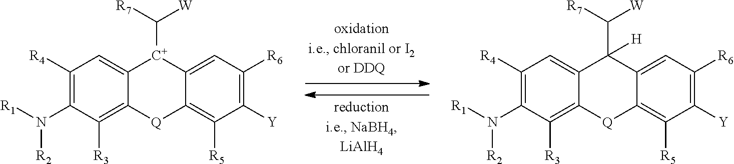

In those embodiments, wherein A=C⁺, the formulas can also be represented as other resonance structures that are equivalents of the original formula (i.e. the positive charge can be located on the N atom or on Y):

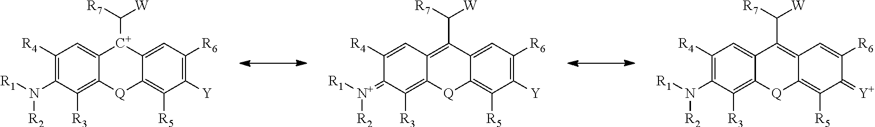

Resonance, also called mesomerism, is a way of describing bonding in certain molecules or polyatomic ions by the combination of several contributing formulas also known as resonance structures. Since in some embodiments of compound X (and its derivatives), the structure cannot be described as a simple Lewis structure due to the delocalization of the electrons, the resonance structures should also be considered. Although in formula (Xa) a single resonance structure is depicted, it is understood that several resonance structures are possible, as shown above, and in fact, the electron system is a combination of these resonance structures.

The distinction of embodiments wherein A=CH (xanthene-derivatives) and A=C⁺ (xanthenium-derivatives) is mainly due to the synthetic access to the photoactivatable conjugates. The synthesis of compounds X requires an intermediate step wherein A=CH, however, such xanthene compounds do not absorb visible light due to the lack of the chromophore moiety. Compounds X wherein A=CH can be either oxidized to compounds X wherein A=C⁺ (xanthenium), forming the visible light absorbing choromophore before attachment to the active agent, or, preferably, can be attached as such to the active agent (or self-immolative linker precursors) forming xanthene-derivatives. In this case the conjugates themselves can be converted to the visible light absorbing xanthenium derivatives, i.e. to the photoactivatable form, via oxidation. In some embodiments of compounds X, wherein A=C⁺ and W═OH it was observed that the stability was insufficient for the formation of the photoactivatable conjugates, therefore compounds X wherein A=CH and W═OH were used for the attachment of the active agent, and the resulting conjugate was oxidized thereafter to the photoactivatable form.

Furthermore, the invention encompasses all protonation states of the compounds X, for example, their deprotonated forms.

More specifically, in those embodiments, wherein A=C⁺ and Y═OH, the deprotonated forms of the original formula, represented by formula (Xd), should be also considered as embodiments of this invention:

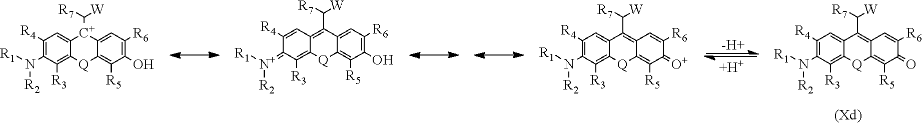

The protonation-deprotonation naturally occurs in aqueous media, and leads to an equilibrium (depending on, among other things, the pH of the environment) between the protonated and deprotonated forms. In case of deprotonation, the counter ion (originally neutralizing the positive charge in X) can be regarded as the counter ion of the leaving proton (i.e. oxonium ion in aqueous media), it is no longer part of compound represented by formula (Xa). The compounds can be isolated from a solution, e.g. from a reaction mixture in either the salt form or in deprotonated form, depending on various factors, especially the pH.

As formulas (Xa) and (Xd) are closely related, in the description the compounds represented by them are commonly referred to as compounds X.

In connection with the above formula, the term “alkyl” means a straight or branched-chain alkyl group containing from 1 to 6, preferably 1 to 4 carbon atom(s), such as methyl, ethyl, propyl, isopropyl, butyl, sec-butyl, t-butyl and pentyl, etc. The alkyl group may be substituted by one or more, preferably by 1 to 3 substituent(s) independently selected from the group of halogen, hydroxyl and CF₃.

The term “alkenyl” means a straight or branched-chain hydrocarbon group containing one or more double bond, and from 2 to 6, preferably 2 to 4 carbon atom(s), such as, vinyl, allyl, 1-butenyl, 2-butenyl, 3-butenyl, 1-pentenyl etc. The alkenyl group may be substituted by one or more, preferably by 1 to 3 substituent(s) independently selected from the group of halogen, hydroxyl and CF₃. Preferably, the alkenyl group is attached via a saturated carbon atom to the rest of the molecule, i.e. it may be for example a 2-allyl, 2-butenyl, 3-butenyl, 4-pentenyl etc.

The term “cycloalkyl” means a monocyclic, saturated hydrocarbon group. Examples include, but are not limited to, cyclopropyl, cyclobutyl, cyclopentyl, cyclohexyl. The cycloalkyl group may be substituted by one or more, preferably by 1 to 3 substituent(s) independently selected from the group of C₁-C₄ alkyl, halogen, hydroxyl and CF₃.

The term “heterocycloalkyl” means a saturated monocyclic group having at least one heteroatom selected from O, N or S, preferably from O and N. Examples of heterocycloalkyl groups include (but are not limited to): azetidinyl, pyrrolidinyl, piperidinyl, piperazinyl, morpholinyl, thiomorpholinyl, azepanyl, diazepanyl, acezanyl group. The heterocycloalkyl groups comprise 3 to 10, preferably 4 to 6 ring members, and may be substituted by one or more, preferably by 1 to 3 substituent(s) independently selected from the group of C₁-C₄ alkyl, halogen, hydroxyl and CF₃.

The term “non aromatic, saturated or unsaturated heterocycle” encompasses the above mentioned heterocycloalkyl groups, as well as unsaturated but non aromatic rings, for example a dihydropyridine ring. The non aromatic, saturated or unsaturated heterocycle comprises a N atom, and optionally contains a further heteroatom selected from N, O and S, and preferably comprises 5 to 7 ring members. In a preferred embodiment, the heterocycle comprises only one N as heteroatom. The heterocycle may be substituted by one or more, preferably by 1 to 5 substituent(s) independently selected from the group of C₁-C₄ alkyl, halogen, hydroxyl and CF₃.

When the heterocycle is formed by any one of the substituent pairs R₃ and R₂, R₄ and R₁, R₅ and R₈, and/or R₆ and R₉, said heterocycle is condensed with the xanthene (or xanthenium) skeleton, and in case two adjacent rings are formed, with each other.

The term “aryl” refers to six to ten-membered aromatic monocyclic or bicyclic hydrocarbon rings, which may be attached via one of the ring carbon atoms. The aryl group may be substituted by one or more, preferably by 1 to 3 substituent(s) independently selected from the group of C₁-C₄ alkyl, halogen and CF₃. Specific examples include, but are not limited to, phenyl, tolyl, xylyl, trimethylphenyl, and naphthyl.

In connection with the above definitions, a preferred halogen substituent is fluorine.

Reference to a compound X herein is understood to include reference to salts thereof, unless otherwise indicated. The term “salt(s)”, as employed herein, denotes salts formed with inorganic and/or organic acids. Pharmaceutically acceptable (i.e., non-toxic, physiologically acceptable) salts are preferred, although other salts are also useful. Said pharmaceutically acceptable salts may be prepared from inorganic and/or organic acids. For example, inorganic acids include hydrochloric acid, hydrobromic acid, hydroiodic acid, sulfuric acid, nitric acid, phosphoric acid, and the like. Organic acids include acetic acid, trifluoroacetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. A preferred salt is an iodide or trifluoroacetate salt.

The term “counter anion” refers to an inorganic or organic anion, which balances the charge of the cationic compound. Pharmaceutically acceptable anions are preferred. Examples of such anions include the following: Cl⁻, Br⁻, I⁻, AcO⁻, CF₃COO⁻, BF₄⁻, PF₆⁻, sulfate, phosphate, methanesulfonate, formate, citrate, fumarate, malate, tartate, succinate, and salicylate. A preferred anion is I⁻.

As used herein, the term “OH-protecting group” or “hydroxyl protecting group” refers to readily cleavable groups bonded to hydroxyl groups. The hydoxyl protecting group can be an acid-labile protecting group, a base-labile protecting group, or a protecting group that is removable under neutral conditions. The nature of the hydroxyl protecting groups is not critical so long as the derivatized hydroxyl group is stable. Examples of OH-protecting groups include C₁-C₆ alkoxymethyl, C₁-C₆ alkyl, C₃-C₆ cycloalkyl, C₂-C₆ alkenyl, aryl, arylmethyl, acetyl, benzoyl, tetrahydropyranyl, tetrahydrofuranyl and tetrahydrothiofuranyl. Further examples of a hydroxyl protecting group are silyl groups, which can be substituted with alkyl (trialkylsilyl), with an aryl (triarylsilyl) or a combination thereof (e.g., dialkylphenylsilyl). Specific examples include, but are not limited to, trimethylsilyl (TMS), triethylsilyl (TES), triisopropylsilyl (TIPS), t-butyldimethylsilyl (TBDMS), t-butyldiphenylsilyl (TBDPS).

Other suitable protecting groups for OH are well known to those of skill in the art (see Wuts, P G M and Greene, TW (2006) “Greene's Protective Groups in Organic Synthesis”, 4th Edition, John Wiley & Sons, Inc., Hoboken, NJ, USA).

Preferred hydroxyl protecting groups are C₁-C₆ alkoxymethyl, C₁-C₆ alkyl, C₃-C₆ cycloalkyl, C₂-C₆ alkenyl, aryl, arylmethyl, acetyl, benzoyl, tetrahydropyranyl, tetrahydrofuranyl, tetrahydrothiofuranyl, and C₁-C₆ alkyl or aryl-substituted silyl groups. More preferred hydroxyl protecting groups are C₁-C₆ alkoxymethyl, C₁-C₆ alkyl, and C₁-C₆ alkyl- or aryl-substituted silyl groups.

In formula (Xa) or (Xd), the substituents are as defined above under the “Summary of the invention”.

In formula (Xa) (and, if applicable, in formula (Xd)), the preferred substituents are as follows.

Q is preferably selected from O and C(R₁₁)₂, wherein R₁₁ is selected from C₁-C₆ alkyl groups. More preferably Q is C(R₁₁)₂ wherein R₁₁ is C₁-C₆ alkyl, for example methyl or ethyl.

R₇ is preferably hydrogen, C₁-C₆ alkyl, C₂-C₆ alkenyl, C₃-C₆ cycloalkyl or (C₆-C₁₀ aryl)(C₁-C₆ alkyl).

In one embodiment, R₇ is C₁-C₆ alkyl, C₂-C₆ alkenyl or C₃-C₆ cycloalkyl.

In another embodiment, R₇ is C₁-C₆ alkyl, C₃-C₆ cycloalkyl or (C₆-C₁₀ aryl)(C₁-C₆ alkyl).

In a further embodiment, R₇ is C₁-C₆ alkyl or C₃-C₆ cycloalkyl.

In yet a further embodiment, R₇ is C₁-C₆ alkyl or C₂-C₆ alkenyl.

In more preferred embodiments, R₇ is C₁-C₆ alkyl.

Preferably, R₁ and R₂ are each selected independently from hydrogen and C₁-C₆ alkyl; or R₁ and R₂, together with the nitrogen atom, to which they are attached, form a 4-6-membered heterocycloalkyl ring containing said N atom, and optionally containing a further heteroatom selected from N and O.

In one embodiment, Y is NR₈R₉, wherein preferably R₈ and R₉ are each independently selected from hydrogen and C₁-C₆ alkyl; or

R₈ and R₉, together with the nitrogen atom, to which they are attached, form a 4-6-membered heterocycloalkyl ring containing said N atom, and optionally containing a further heteroatom selected from N and O.

In another embodiment, Y is OR₁₀, wherein R₁₀ is selected from hydrogen and OH-protecting group.

Preferably, R₃, R₄, R₅, R₆ are each selected independently from H and C₁-C₆ alkyl.

The substituents R₃ and R₂, R₄ and R₁, and in case Y is NRsR₉, R₅ and R₉, and/or R₆ and R₈ together with the intervening atoms, may form a 5 to 7, preferably 6-membered non aromatic, saturated or unsaturated heterocycle containing a N atom and optionally containing a further heteroatom preferably selected from N and O, said heterocycle being optionally substituted with 1-6 C₁-C₄ alkyl. Most preferred heterocycles are piperidine and dihydropyridine rings that are optionally substituted.

Preferably, R₁₀ is selected from hydrogen, C₁-C₆ alkoxymethyl, C₁-C₆ alkyl, C₃-C₆ cycloalkyl, C₂-C₆ alkenyl, aryl, arylmethyl, acetyl, benzoyl, tetrahydropyranyl, tetrahydrofuranyl, tetrahydrothiofuranyl, and C₁-C₆ alkyl or aryl-substituted silyl groups. More preferably, R₁₀ is selected from hydrogen, C₁-C₆ alkoxymethyl, C₁-C₆ alkyl, and C₁-C₆ alkyl- or aryl-substituted silyl groups.

In one embodiment, W is selected from
OH, and
halogen, preferably bromine.

In another embodiment, W is selected from
an oxycarbonyl linker precursor moiety, preferably selected from:

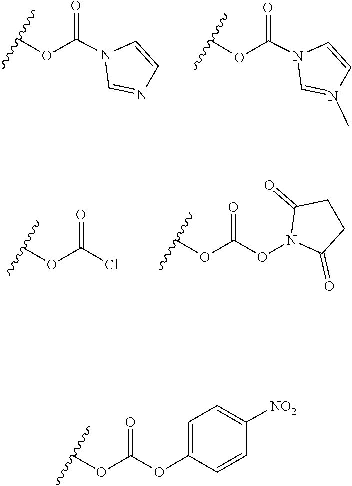

or a self-immolative linker precursor moiety, preferably selected from:

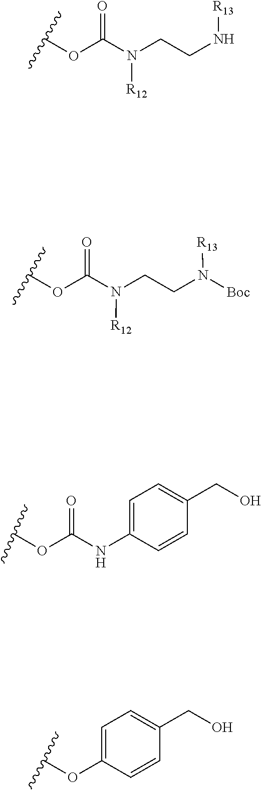

wherein R₁₂ and R₁₃ are independently selected from hydrogen, C₁-C₆ alkyl, C₃-C₆ cycloalkyl, C₂-C₆ alkenyl; or R₁₂ or R₁₃, together with the nitrogen atom to which they are attached and with one or both of the carbon atoms of the ethylene moiety, form a 4-6 membered heterocycloalkyl containing said N atom, and optionally containing a further heteroatom selected from N, O and S, and the wavy line ( [symbol] ) represents the attachment point to the rest of the molecule. More preferably, R₁₂ and R₁₃ are independently selected from hydrogen, C₁-C₆ alkyl groups; or R₁₂ or R₁₃, together with the nitrogen atom to which they are attached and with one or both of the carbon atoms of the ethylene moiety, form a 5-membered heterocycloalkyl containing said N atom.

We note that compounds represented by formula (Xa) or (Xd) wherein W represents a linker precursor group can be conveniently prepared from compounds represented by formula (Xa) or (Xd) wherein W represents an OH group or halogen.

Preferably, when A is C⁺, the counter anion is selected from Cl⁻, Br⁻, I⁻, HCOO⁻, AcO⁻, CF₃COO⁻, BF₄⁻, PF₆⁻, methanesulfonate, sulfate, phosphate or citrate.

In the following, some preferred sub-structures of the compounds X are described. These are also subject matters of the invention.

1.2 Rhodamine-Like Structures (Y═NR₈R₉)

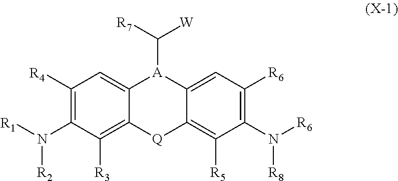

wherein A, Q, W, and R₁ to R₉ are as defined above.

### 1.3 Rhodol-Like Structures (Y═OR₁₀)

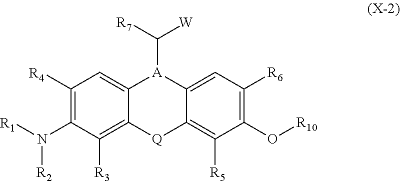

wherein A, Q, W, and R₁ to R₇ and R₁₀ are as defined above.

Preferred meanings of R₁₀ include hydrogen, C₁-C₆ alkoxyalkyl or C₁-C₆ alkyl, more preferably alkoxymethyl.

In those embodiments, wherein A=C⁺ and R₁₀=hydrogen, the deprotonated forms of the original formula are also included herein and should be also considered as embodiments of this invention.

1.4 O-rhodamine-like structures (Y═NR₈R₉, Q=O)

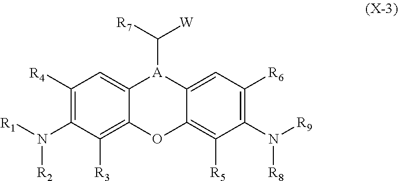

wherein A, W, and R₁ to R₉ are as defined above.

### 1.5 O-Rhodol-Like Structures (Y═OR₁₀, Q=O)

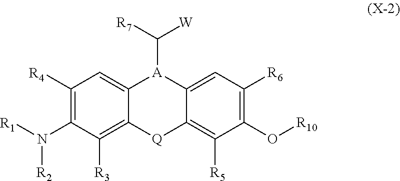

wherein A, W and R1 to R₇ and R₁₀ are as defined above.

Preferred meanings of R₁₀ include hydrogen, C₁-C₆ alkoxyalkyl or C₁-C₆ alkyl, more preferably alkoxymethyl.

In those embodiments, wherein A=C⁺ and R₁₀=hydrogen, the deprotonated forms of the original formula are also included herein and should be also considered as embodiments of this invention.

1.6 Carborhodamine-Like Structures (Y═NR₈R₉, Q=CR₂)

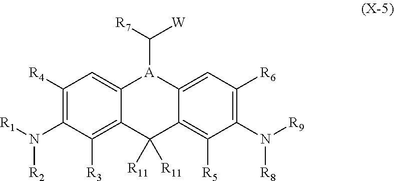

wherein A, W and R₁ to R₉ and R₁₁ are as defined above.

Preferred meanings of R₁₁ include C₁-C₆ alkyl, more preferably methyl.

### 1.7 Specific Structures for Compounds Represented by Formula (Xa) (A=CH, W═OH)

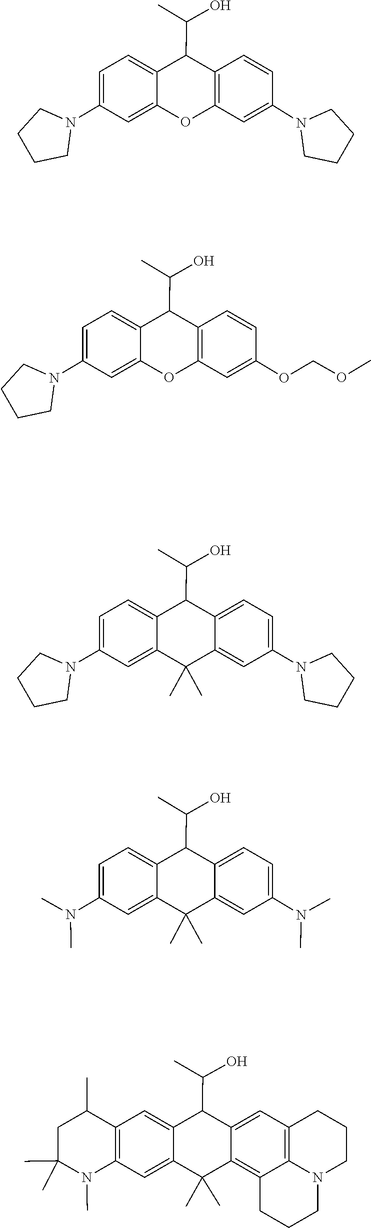

### 1.8 Specific Structures for Compounds Represented by Formula (Xa) (A=C+, W═OH)

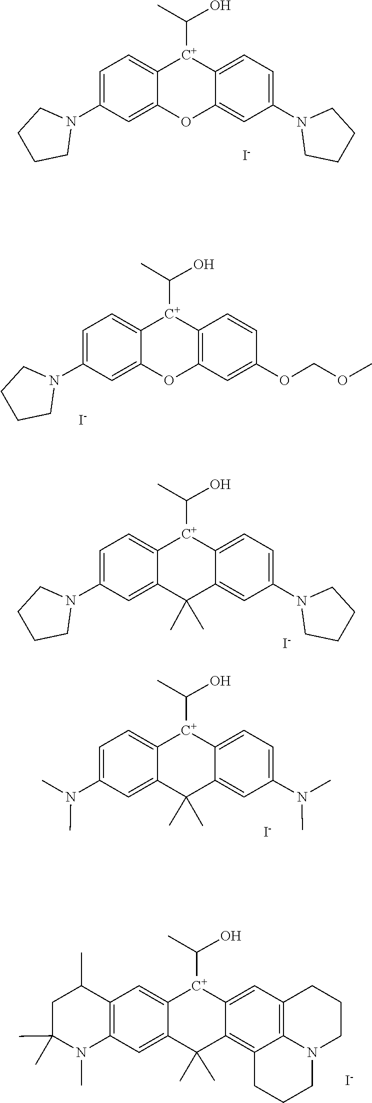

### 1.9 Specific Structures for Compounds Represented by Formula (Xa) (A=CH, W=Linker Precursor Moiety)

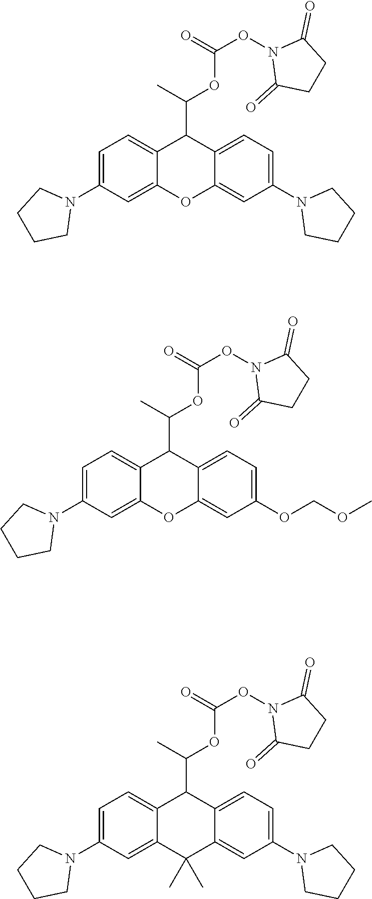 [SMILES: CC(OC(=O)ON1C(=O)CCC1=O)C1c2ccc(N3CCCC3)cc2Oc2cc(N3CCCC3)ccc21 ; COCOc1ccc2c(c1)Oc1cc(N3CCCC3)ccc1C2C(C)OC(=O)ON1C(=O)CCC1=O ; CC(OC(=O)ON1C(=O)CCC1=O)C1c2ccc(N3CCCC3)cc2C(C)(C)c2cc(N3CCCC3)ccc21]

### 1.10 Specific Examples for Target Conjugates: X′-SN38

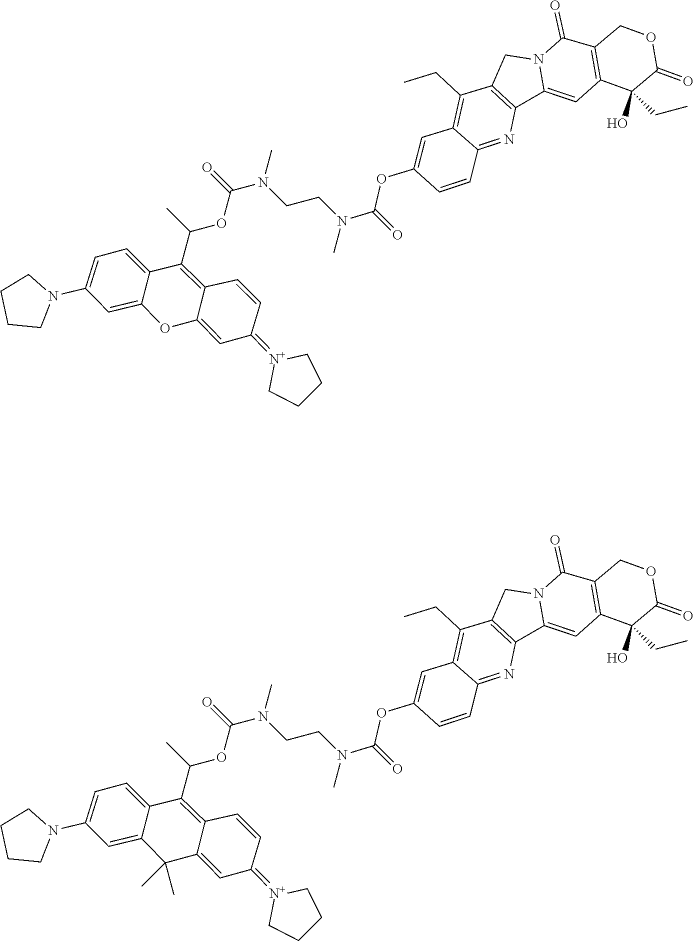 [SMILES: CCc1c2c(nc3ccc(OC(=O)N(C)CCN(C)C(=O)OC(C)c4c5ccc(=[N+]6CCCC6)cc-5oc5cc(N6CCCC6)ccc45)cc13)-c1cc3c(c(=O)n1C2)COC(=O)[C@]3(O)CC ; CCc1c2c(nc3ccc(OC(=O)N(C)CCN(C)C(=O)OC(C)C4=C5C=CC(=[N+]6CCCC6)C=C5C(C)(C)c5cc(N6CCCC6)ccc54)cc13)-c1cc3c(c(=O)n1C2)COC(=O)[C@]3(O)CC]

### 2. Connecting Chemical Moieties Between the Photoremovable Protecting Groups and Cargo Units (Moiety L from X′-L-Z′)

The connecting chemical moiety or linker (L) that connects the cargo unit and the photoremovable protecting group is largely dependent on the selected functional group on the active agent that is to be blocked via the attachment of compound X. Usually, the functional group (i.e., NH₂, COOH, OH, etc.) on the active agent that is selected to be caged by photoremovable protecting group X′ is an essential element that interacts with the target of the agent (i.e., an H-donor group in the binding pocket of the target protein of a drug). In the present invention, the variability of functional groups that can be blocked by the attachment of compound X is demonstrated by exemplary target conjugates featuring different linkers and cargo units (in other words, caged functional groups). The formation of the target conjugates from compound X is also disclosed herein.

In the target conjugates, the photoremovable protecting group X′ derived from compound X has the one of the following formulas:

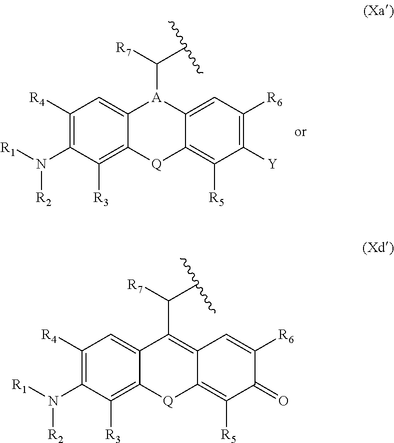

wherein the wavy line ( [symbol] ) represents the attachment point to L,
A=C⁺
Q, Y and R₁ to R₇ are as defined above.

We note that in the target conjugates, the photoremovable protecting group is also a chromophore (having absorption bands in the visible range), therefore in the above formula (Xa′) A=C⁺, meaning that (Xa′) derived from compound represented by formula (Xa) is in its xanthenium form or oxidized state. As formula (Xd) is the deprotonated form of the oxidized form of compounds represented by formula (Xa) wherein Y═OH, the group derived therefrom, represented by formula (Xd′), is also in the oxidized state, i.e. in the photoactivatable form.

### 2.1 Covalent Bond

In one embodiment, the connecting chemical moiety (L) is a covalent bond that connects the photoremovable protecting group (X′) and the cargo unit (Z′) by the replacement of moiety W in compound X with the cargo unit. Such target conjugates can be prepared via direct or indirect chemical methods of connecting a compound X wherein W is OH or halogen, with the active agent, followed by an oxidation step in case of A=CH.

Upon absorbing visible light, this connecting covalent bond is cleaved. Upon cleavage, in this embodiment the active agent is released directly.

In this embodiment, the functional groups on the active agent that may be connected to compound X via a covalent bond include, but are not restricted to hydroxyl groups (ethers in the target conjugates), primary, secondary and tertiary amino groups (secondary, tertiary and quaternary amines or ammoniums in the target conjugates), thiol groups (thioethers in the target conjugates), carboxyl or carboxylate groups (esters in the target conjugates), pyridyl groups (pyridiniums in the target conjugates), phosphate groups (phosphate esters in the target conjugates).

The replacement of moiety W in the general formula of compound X with the cargo unit and its subsequent photolysis can be better understood from the following scheme, wherein the wavy line ( [symbol] ) represents the attachment point of the cargo unit to the photoremovable protecting group:

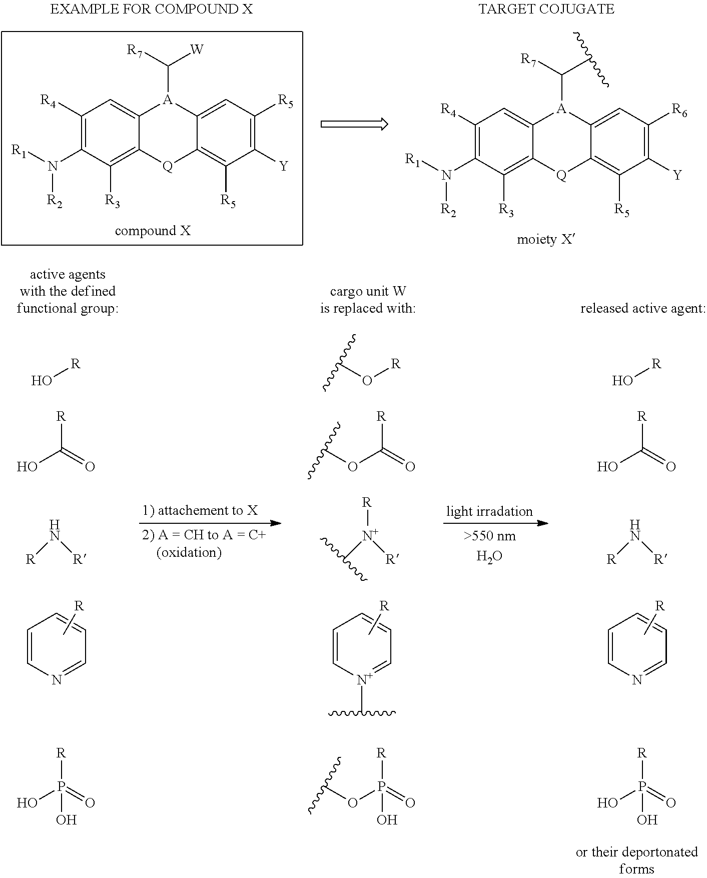

The following examples illustrate the formation of target conjugates (using model compounds with different functional groups) and their subsequent photolysis wherein the connecting chemical moiety is a chemical bond:

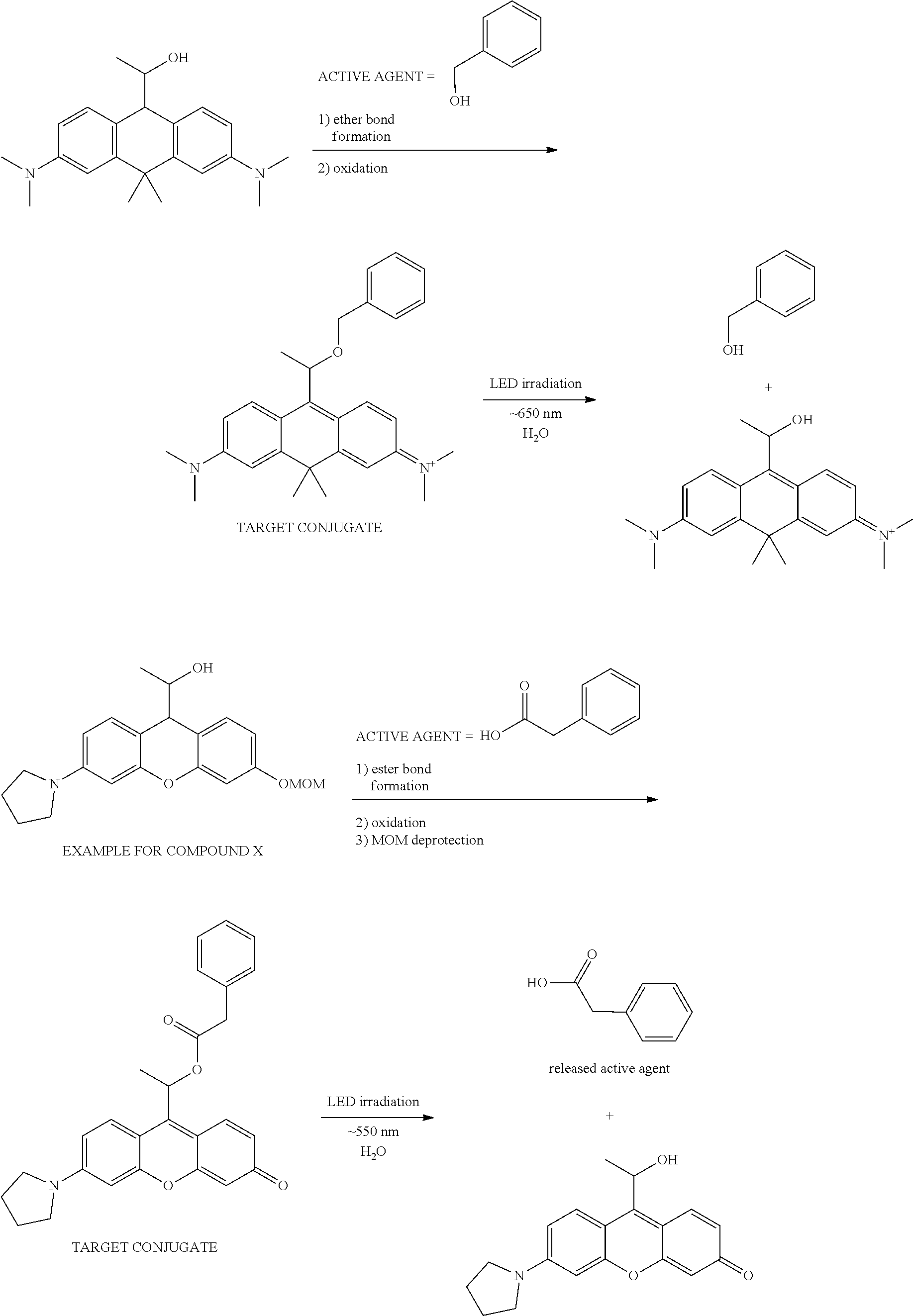

### 2.2 Oxycarbonyl Linker

In another embodiment, the connecting chemical moiety between the photoremovable protecting group 5 (X′) and the cargo unit (Z′) in the target conjugates can be an oxycarbonyl linker, such as —O—(C═O)— or —O—(C═S)—, wherein the oxygen is connected to X′ and the carbonyl/thiocarbonyl carbon atom is connected to either an oxygen or a nitrogen atom of Z′. If the connecting atom on Z′=oxygen (originating from a hydroxyl functional group of Z), the resulting chemical moiety is called a carbonate with the formula —O—(C═O)—O—, or thiocarbonate with the formula —O—(C═S)—O—. If the connecting atom on Z′=nitrogen (originating from an amino functional group of Z), the resulting chemical moiety is called a carbamate with the formula —O—(C═O)—N—, or thiocarbamate with the formula —O—(C═S)—N—.

These compounds can be prepared by general chemical methods capable of connecting compound X (wherein W═OH) with the active agent through such moieties. These methods include the following steps.

First, the activation of compound X wherein W═OH with phosgene, thiophosgene, triphosgene, N,N′-carbonyldiimidazole, N,N′-dissucinimidyl carbonate, 4-nitrophenyl chloroformate, etc. This results in compounds wherein moiety W is an oxycarbonyl linker precursor.

The second step is the reaction of the resulting activated species with the active agent or its protected form. This is followed by an oxidation step in case of A=CH in the first step.

Alternatively, the active agent can also be activated with phosgene, thiophosgene, triphosgene, N,N′-carbonyldiimidazole, N,N′-dissucinimidyl carbonate, 4-nitrophenyl chloroformate, etc. The resulting activated compound (a derivative of the active agent) reacts with compound X wherein W═OH forming the target conjugate containing an oxycarbonyl linker. This is followed by an oxidation step in case of A=CH in compound X.

In either case, the resulting target conjugate releases an unstable derivative of the active agent in its s anionic form (i.e., —(C═O)—O⁻ adducts of the cargo units) upon absorbing visible light, that rapidly releases a molecule of CO₂ and the free active agent with restored activity.

The formation of target conjugates wherein the connecting chemical moiety is an oxycarbonyl linker and their subsequent photolysis can be better understood from the following scheme wherein the wavy line ( [symbol] ) represents the attachment point of the linker to the photoremovable protecting group (X′) and the asterisk ( [symbol] ) represents the attachment point of the cargo unit to the linker:

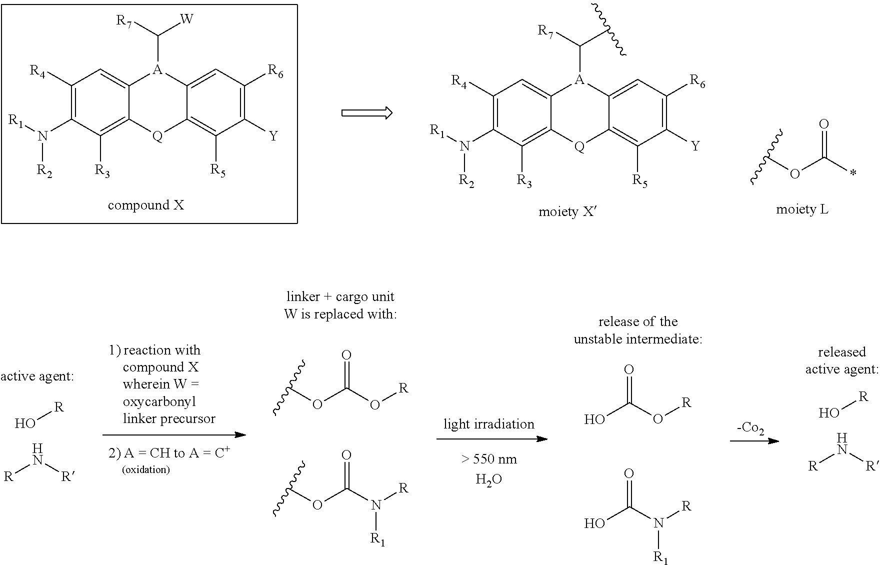

The following examples illustrate the connection formation between compounds X and active agents (using model compounds with different functional groups) and the subsequent photolysis of the resulting target conjugates wherein the connecting chemical moiety is an oxycarbonyl linker:

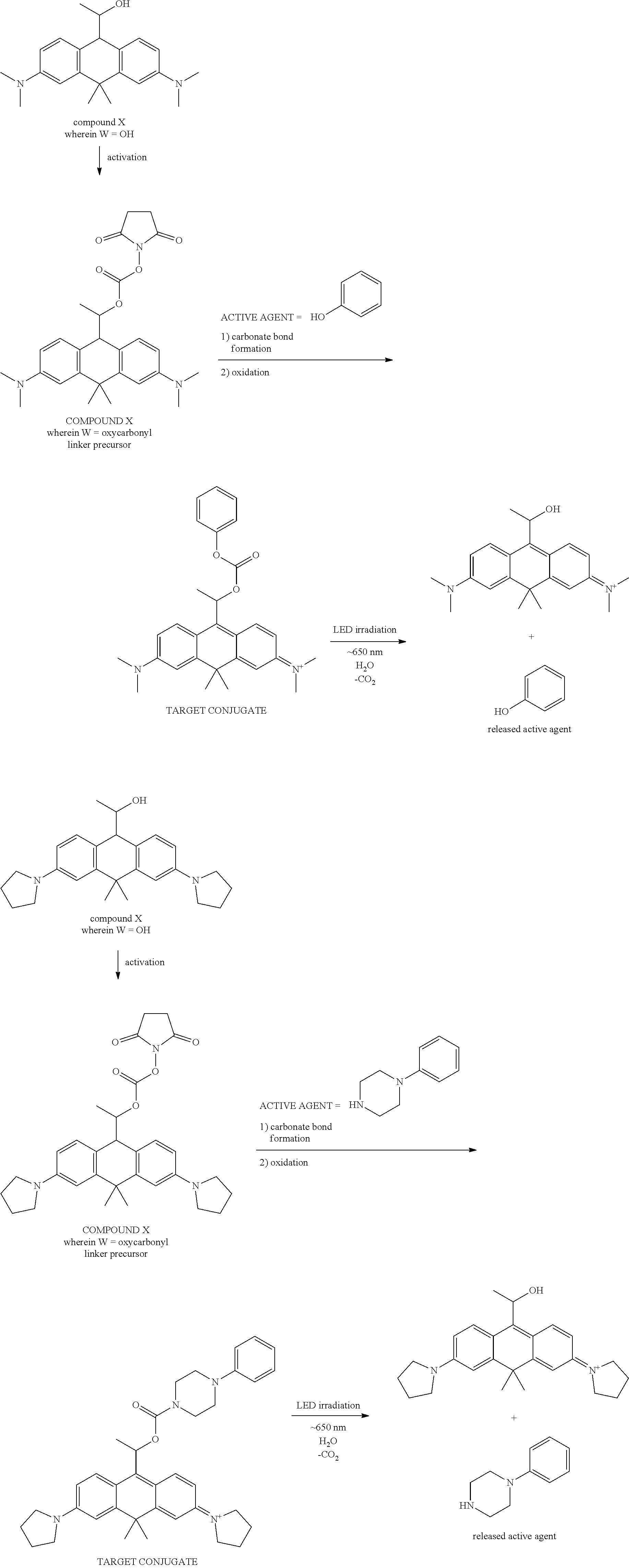

### 2.3 Self-Immolative Linker

In a further embodiment, the connecting chemical moiety (L) between the photoremovable protecting group (X′) and the cargo unit (Z′) in the target conjugates can be a self-immolative linker, which, may have, for example one of the following formulas:

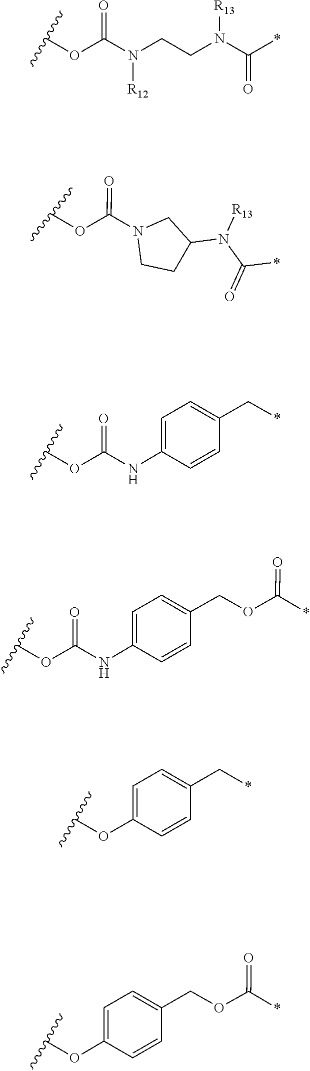

wherein
the wavy line ( [symbol] ) represents the attachment point to the photoremovable protecting group X′;
the asterisk ( [symbol] ) represents the attachment point to the cargo unit Z′;
R₁₂ and R₁₃ are as defined above.

These target conjugate compounds can be prepared via several steps.

First, a compound X, wherein W is OH or halogen, is converted to a compound X, wherein W is a self-immolative linker precursor moiety. Exemplary compounds that are useful for formation of a self-immolative linker (or its precursor moiety) are ethylenediamine derivatives, for example N,N′-dimethylethylenediamine, N,N′-diethylethylenediamine, N-Boc-N,N′-dimethylethylenediamine, hydroxymethylphenol derivatives or hydroxymethylaniline derivatives, for example 4-hydroxymethylphenol, 2-hydroxymethylphenol, 4-hydroxymethylaniline, 2-hydroxymethylaniline, 4-hydroxymethyl-N-methylaniline. These compounds can be attached to X (wherein X═OH) via direct covalent bond formation (as described in section 2.1) or via an oxycarbonyl linker (as described in section 2.2).

An exemplary structure of self-immolative linker precursor moieties that are attached through direct covalent bond to X′ is:

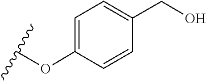 [SMILES: *Oc1ccc(CO)cc1]

Exemplary structures of self-immolative linker precursor moieties that are attached through an oxycarbonyl linker to X′ are:

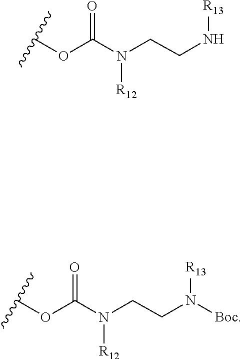

Next, the obtained compound X wherein W is a self-immolative linker precursor moiety can be connected to the cargo unit through either direct covalent bond formation (as described in section 2.1) or via an oxycarbonyl linker formation (similarly as described in section 2.2, wherein either the free end of the linker precursor moiety or the selected functional group of the active agent can be activated for the oxycarbonyl linker formation).

Exemplary structure of self-immolative linkers that are attached through direct covalent bond to the cargo unit Z′ are:

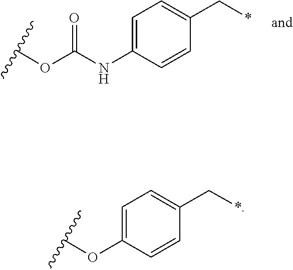

Exemplary structure of self-immolative linkers that are attached through an oxycarbonyl linker to the cargo unit Z′ are:

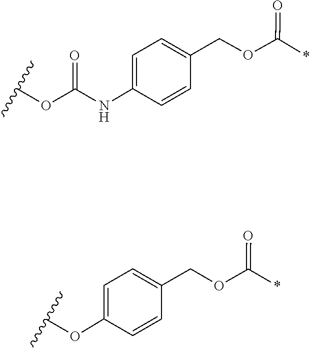 [SMILES: *OC(=O)Nc1ccc(COC(*)=O)cc1 ; *Oc1ccc(COC(*)=O)cc1]

Alternatively, the linker precursor moiety can be attached to the active agent in the first step, and thereafter the resulting structure can be attached to X (wherein X═OH). Direct covalent bond formation or oxycarbonyl linker formation can be applied in each step, similarly as described above.

Finally, an oxidation step is carried out in case of A=CH in compound X.

The resulting target conjugate, upon absorbing visible light, releases a derivative of the active agent that also contains a moiety derived from the self-immolative linker as an unstable chemical moiety. This drives a reaction cascade that transforms that moiety into an organic byproduct on the one hand and releases the free active agent on the other. In these target conjugates, the advantages of using a self-immolative linker are the larger distance between the photoremovable protecting group and the cargo unit and that it allows the transformation of the connecting chemical moiety to a physiologically more stable element, e.g., a carbonate to two carbamate groups.

The following examples demonstrate some possible embodiments of the connection between the linker-appended compounds X and cargo units and their subsequent photolysis wherein the connecting chemical moiety is a self-immolative linker:

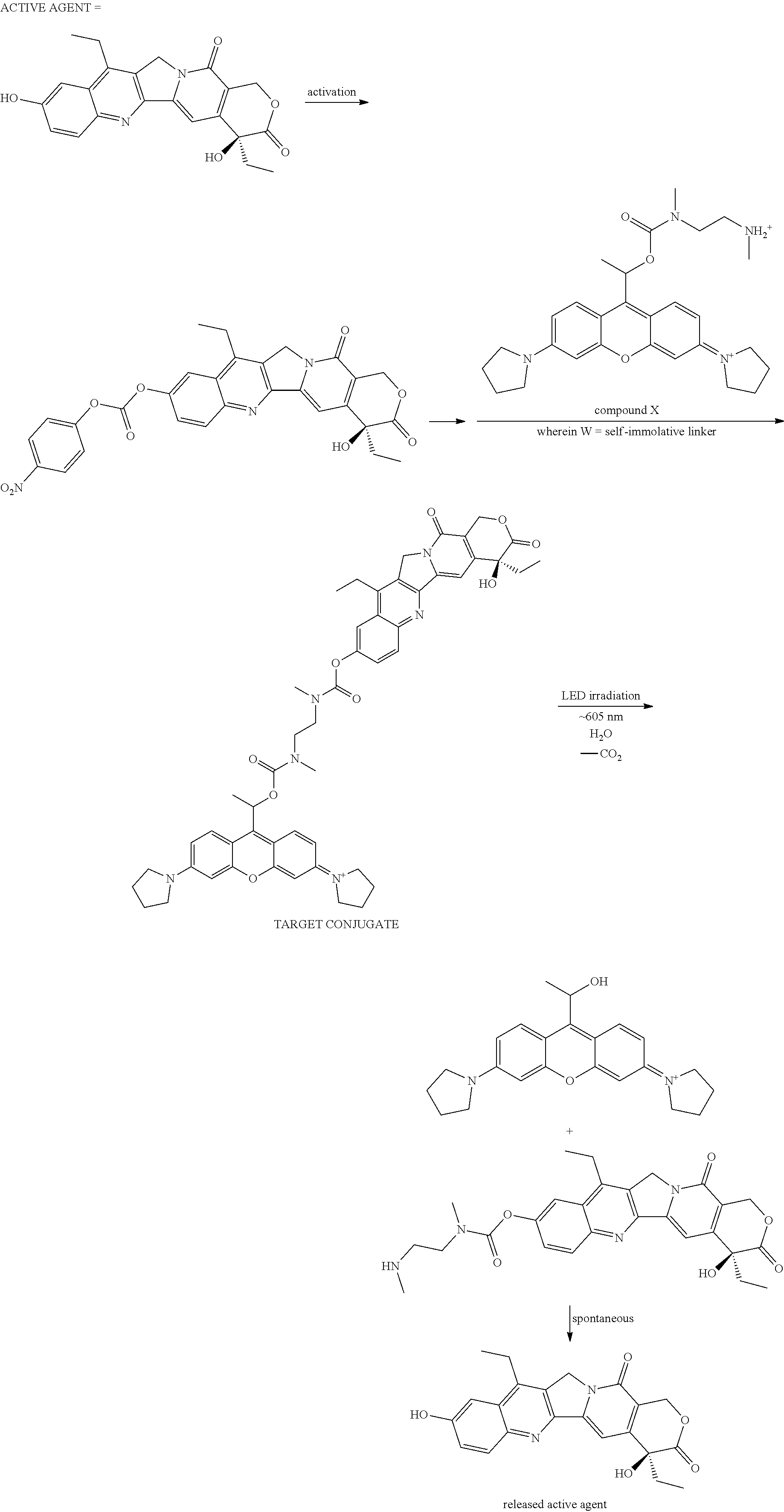

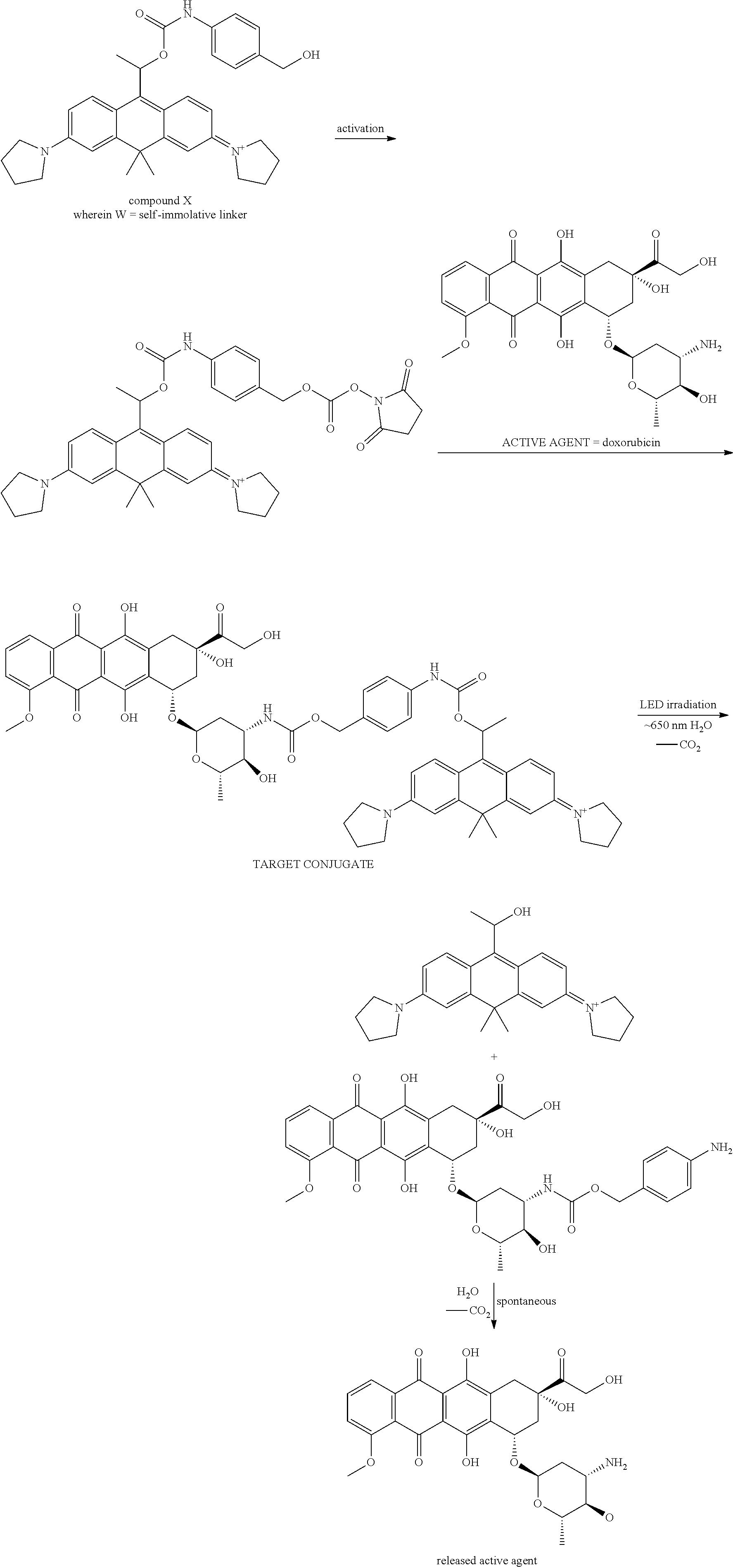

### 3. Synthesis of Compounds X

We note that our initial attempts included the conversion of substituted xanthones to their 9-hydroxymethyl derivatives, however, it became evident that the oxidized 9-hydroxymethyl scaffolds are unstable. Following attachment of the cargo moiety, we could only isolate a mixture of products, which impeded the utilization of the target compound. Notably, even after subsequent purification steps, the target compound degraded significantly indicating that the 9-hydroxymethyl derivatives are not suitable as precursors of PPGs.

Strikingly, we have found that this problem can be solved by introducing an additional substituent onto the carbon atom adjacent to the the 9-position of the xanthene scaffold (i.e., the carbon atom bearing the leaving group or cargo moiety). Compounds X of the present invention, bearing an R₇ substituent (other than a hydrogen atom) on the carbon atom adjacent to the the 9-position of the xanthene scaffold, can be readily synthesized, isolated and attached to cargo moieties. Some possible synthetic routes are set forth in the following sections.

3.1 Synthesis of Compounds X from Xanthone Precursors Wherein A=CH and W═OH

As mentioned above, the compounds X can be readily prepared from xanthone precursors, known extensively in the literature. These starting compound(s) can be prepared from readily accessible materials using methods described in the relevant literature.³⁵⁻³⁸ The compound X wherein A=CH and W═OH can be prepared either by a Grignard reaction followed by hydroboration-oxidation or using an umpolung strategy. The general scheme of the first strategy is depicted in the following scheme:

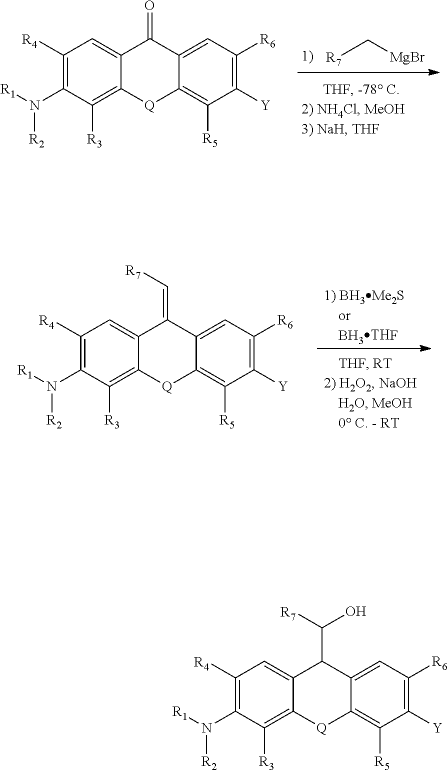

In detail, the starting xanthone compound is first reacted with a Grignard reagent such as ethylmagnesium bromide. Next, the resulting adduct is converted to an olefin derivative using first ammonium chloride and then deprotonation using a strong base, such as NaH. The resulting compound features an exo double bond that can be further functionalized using hydroboration-oxidation (using a borane reagent then hydrogen peroxide) that enables the anti-Markovnikov addition of a hydroxyl group and a hydrogen resulting in compound X wherein A=CH and W═OH.

The second method that is disclosed uses an acyl anion umpolung strategy and is depicted in the following scheme:

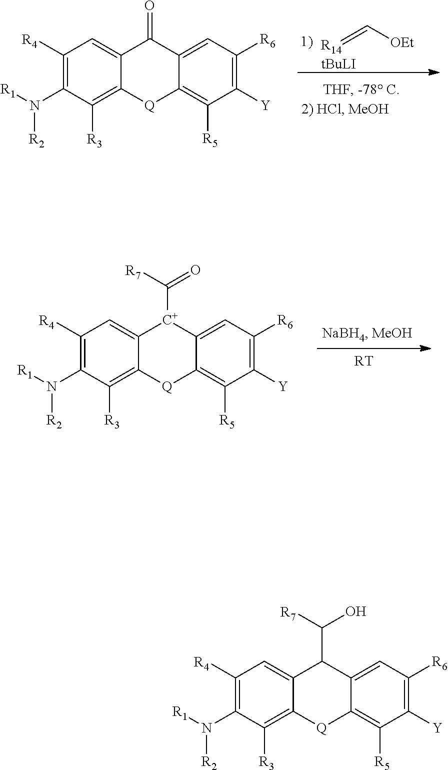

In detail, the starting xanthone compound is reacted with a deprotonated vinyl ether derivative which is preformed by treatment of a vinyl ether of formula R₁₄CH-OAlk with a strong base (using e.g. tert-butyl lithium), wherein R₁₄ represents e.g. CH₂═ or CH₃—(CH₂)ₙ—CH═ wherein n is an integer from 0 to 5, wherein said R₁₄ can be considered as a precursor group of R₇ and Alk refers to an alkyl group. This results in the formation of a vinyl-ether appended xanthenium derivative that can be hydrolyzed to the acyl intermediate (during vinyl ether hydrolysis, R₇—C(═O)— is formed from R₁₄═CH—O— due to enol-oxo tautomerism). Reduction of the carbonyl and the chromophore frame results in compounds wherein A=CH and W═OH. Note that the resonance structures of the acyl intermediate are also considered herein.

As it can be seen in the examples, this method gave quantitative yields in case of several structures, without the need for any subsequent purification by chromatography.

Although multiple synthetic pathways can be envisaged by those skilled in the art for the formation of compounds X, the present inventors realized that the particularly important requirement for late-stage cargo attachment to the PPG is strongly dependent on the PPG precursors (compounds X). Therefore, the synthetic strategy towards the target conjugates were selected to be as modular as possible. Such modular access includes the primary formation of compounds X then its derivative with the connecting chemical moiety and subsequent cargo attachment in the last stage.

### 3.2 Synthesis of Compounds X Wherein A=C⁺ and W═OH

The xanthene-derived compounds X wherein A=CH can be converted to their respective xanthenium derivatives (wherein A=C⁺) as depicted in the following schemes:

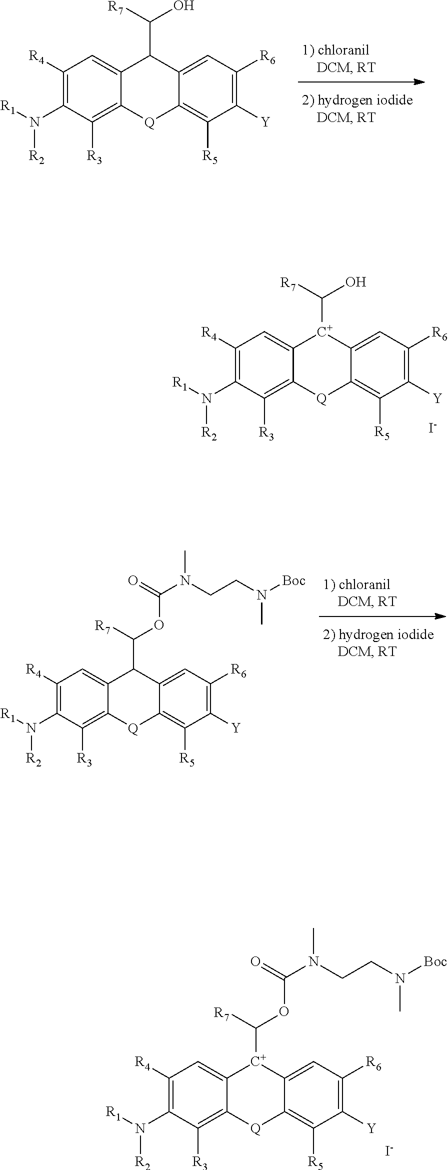

In detail, regardless of moiety W, the reduced xanthene form can be oxidized with, for example, chloranil.

Formal removal of a hydride ion from the xanthene ring results in the formation of the xanthenium chromophore. An ion exchange to iodide using, for example, hydrogen iodide, is usually beneficial for the stability of the xanthenium derivatives. Note that compounds X wherein A=C⁺ and W═OH are usually unstable for longer time periods, therefore their reduced form (A=CH) are more suitable for chemical derivatization in the subsequent steps. In addition, the handling of the xantheniums (A=C⁺) is more complicated due to the required darkness in the laboratory.

### 3.3 Synthesis Compounds X Wherein A=C⁺ and W=Halogen

The compounds X wherein W=halogen can be synthesized from xanthone precursors as depicted in the following scheme:

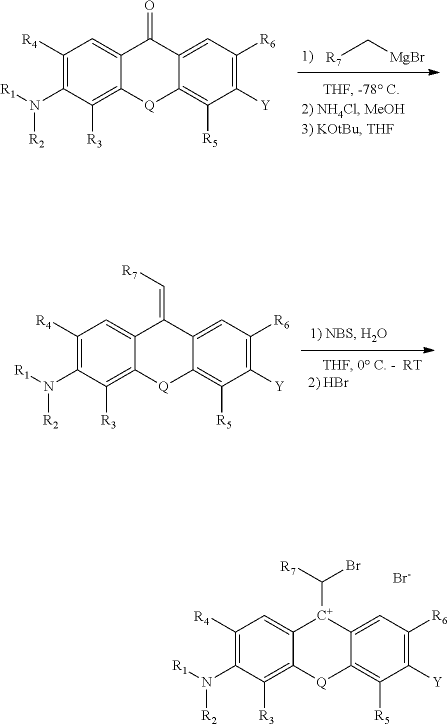

In detail, the starting xanthone compound is first reacted with a Grignard reagent such as ethylmagnesium bromide. Next, the resulting adduct is converted to an olefin derivative using first ammonium chloride and then deprotonation using a strong base, such as NaH or KOtBu. Next, the olefin is reacted with N-bromosuccinimide in the presence of water for the addition of HOBr to the olefin bond. After the elimination of a hydroxide ion, the bromoethyl compound X is formed. Note that N-bromosuccinimide can be replaced with N-chlorosuccinimide or N-iodosuccinimide as well to produce compounds X wherein W═Cl or I.

### 3.4 Synthesis Compounds X Wherein W=Oxycarbonyl Linker Precursor Moiety

The compounds X wherein W═OH can be converted to compounds X wherein W=oxycarbonyl linker precursor moiety using carbonic acid-derived activation reagents such as DSC or 4-nitrophenyl chloroformate as depicted in the following schemes:

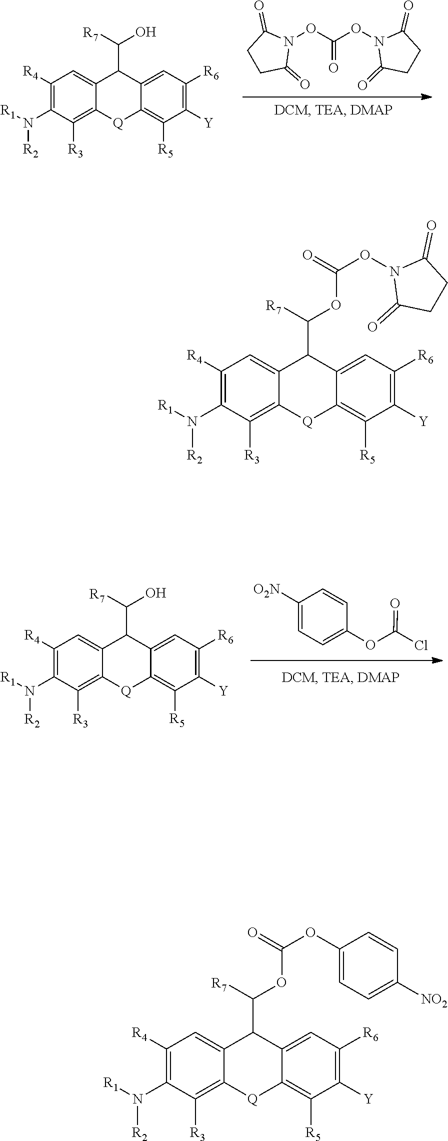

In detail, the OH group in compound X (W═OH) reacts with the carbonic-acid derived DSC or chloroformic acid-derived 4-nitrophenyl chloroformate in the presence of a base such as triethylamine to obtain ‘mixed carbonate’ compounds. These compounds are reactive towards nucleophiles such as amine or alcohol functional groups on the active agents in the presence of a base. Note that these embodiments are just examples of compounds X and as such they do not restrict the scope of the compounds.

### 3.5 Synthesis of Compounds X Wherein W=Self-Immolative Linker Precursor Moiety

The compounds X wherein W=self-immolative linker precursor moiety can be prepared for example, from compounds X wherein W=oxycarbonyl linker precursor moiety as depicted in the following schemes:

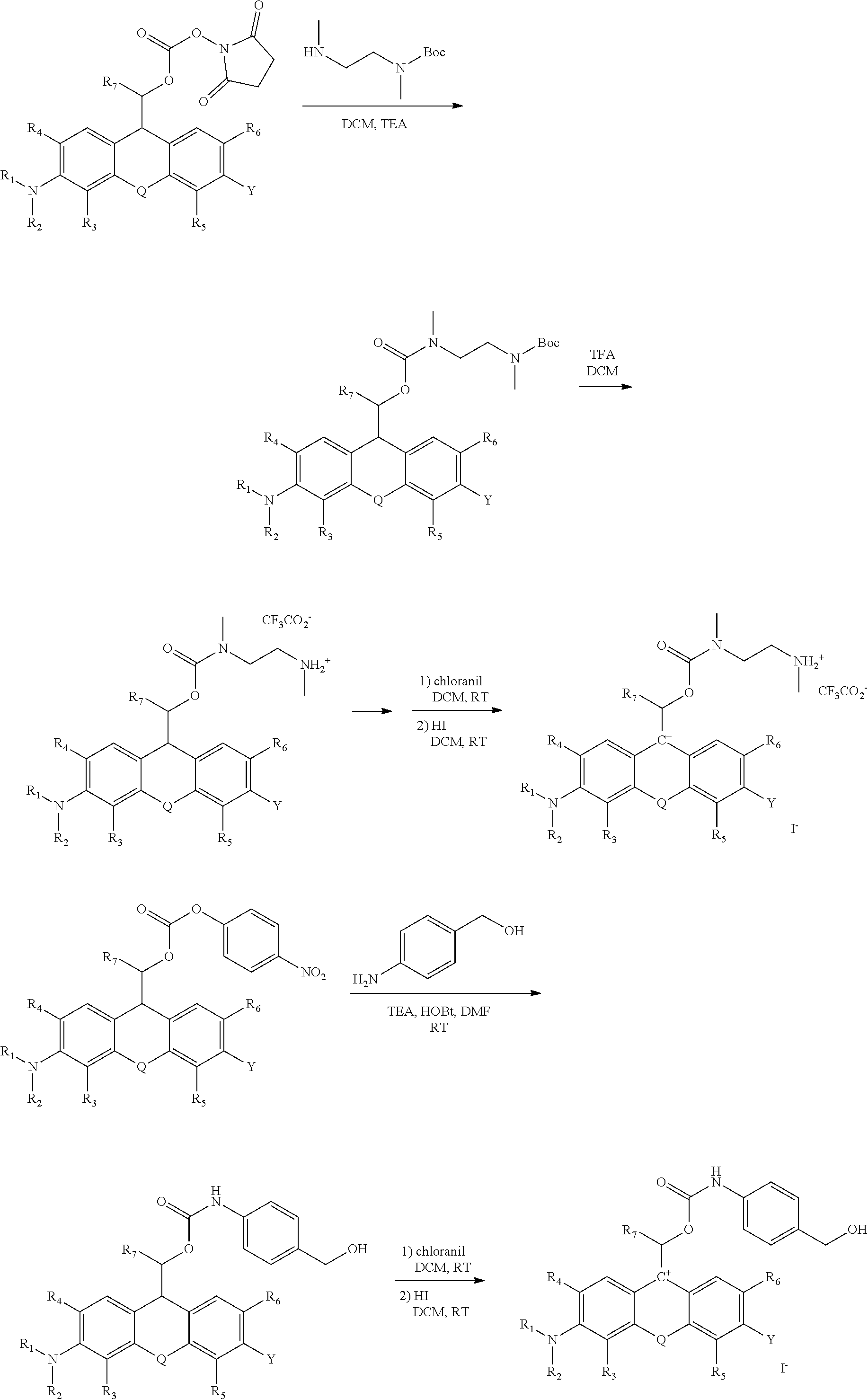

In detail, the carbonate-derivatized compounds X are reacted with either N-Boc-N,N′-dimethylethylenediamine in the presence of a base or 4-hydroxymethylaniline in the presence of a base and 1-hydroxybenzotriazole to form compounds X wherein W=self-immolative linker precursor moiety and A=CH. These compounds can be oxidized to form compounds X wherein W=self-immolative linker precursor moiety and A=C⁺ using chloranil and subsequent anion replacement.

### 4. Application of Compound X as Photoremovable Protecting Group for Manipulating Biological Systems with Light

The compounds of the present invention and the target conjugates can be used to study and manipulate biological systems and how those systems react when the active agent is photoreleased from the target conjugates prepared from compound X.

Upon irradiation with visible light with a wavelength >550 nm, the target conjugates can photorelease the active agents. In other words, attachment of compound X to the active agent via the chemical connecting moiety renders it inactive in their bound form (as cargo units). The active agents therefore become photoactivatable in the target conjugates.

The various active agents used in a biological systems include but are not limited to any chemically/biologically active agent, including an oligopeptide, protein, enzyme, nucleoitide, nucleic acid, carbohydrate, lipid, neurotransmitter, catalyst, fragrance, pharmaceutically active ingredient or drug, anticancer agent, antibiotics, chemotherapeutic agent, small molecule inhibitor, fluorescent dye or fluorogenic dye. Preferably, the active agents are selected from cytotoxic chemotherapeutic agents or inhibitors used for the destruction of cancer cells including but not limited to nucleoside analogs such as cytarabine or fluorouracil, antifolates such as methotrexate, topoisomerase inhibitors such as campthothecin, irinotecan or SN38, anthracyclines such as doxorubicin, daunomycin or mitoxantrone, taxanes such as paclitaxel or docetaxel, vinca alcaloids such as vincristine and vinblastine, alkylating agents such as chlorambucil, melphalan or cyclophosphamide, platinum compounds such as carboplatin or cisplatin, or targeted antineoplastic agents such as tyrosine kinase inhibitors ibrutinib or dasatinib, histone deacetylase inhibitor vorinostat, or other agents such as methylprednisolone, retinoids, thalidomide.

The target conjugate, prepared from the attachment of compound X to an active agent can be used in a biological sample with external irradiation that enables the photorelease of the active agent from the target conjugates, using visible light with a wavelength >550 nm in a spatiotemporally highly controlled fashion. For example, an LED or laser light source can be used as a controlled external stimulus to trigger the photorelease of the active agent from the target conjugate. Upon the photorelease of the active agent, its original activity reinstates in a spatiotemporally highly controlled fashion.

The term “biological sample” includes but not restricted to in vitro cell cultures, e.g. dedicated or selected cells in a microscope, live cells, fixed cells, 3D cell cultures such as spheroids or organoids, tissue samples and preparations, xenograft tumor models or live animals such as mice.

More specifically, the target conjugate can be used in cells with irradiation that enables the photorelease of the active agent, using visible light with a wavelength >550 nm in a spatiotemporally highly controlled fashion, e.g., in dedicated or selected cells in a microscope. In such embodiments, the photoreleased active agents include but not restricted to a fluorescent dye, small molecular effector or a pharmaceutical agent capable of triggering a biological response or signaling event in a spatiotemporally highly controlled fashion. Upon photorelease of the active agent, its original activity reinstates in a spatiotemporally highly controlled fashion.

In another embodiment, the active agent consists of a pharmaceutical agent, chemotherapeutic agent or small molecule inhibitor and can be photoreleased from the target conjugate using visible light with a wavelength >550 nm in a spatiotemporally highly controlled fashion in live cells. Upon photorelease of the active agent, its original activity reinstates in a spatiotemporally highly controlled fashion. For example, the target conjugate can include but not restricted to compounds resulting from the attachment of compound X to a derivative of SN38 via a self-immolative linker as depicted here:

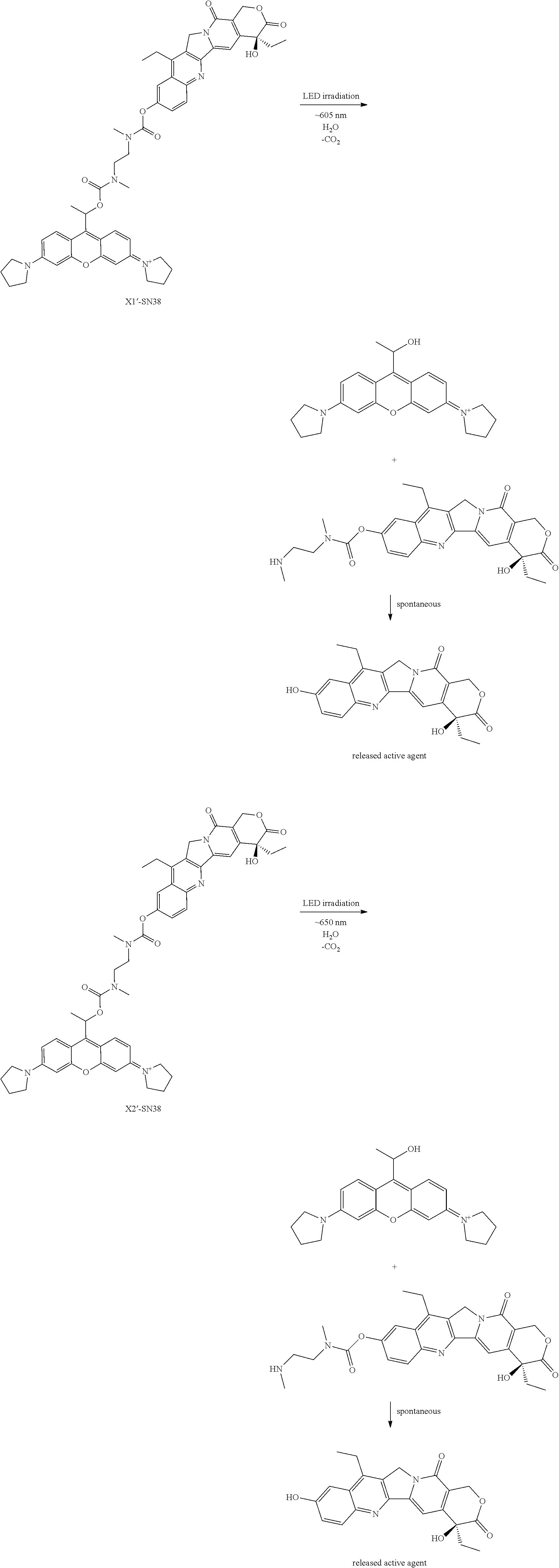

The above mentioned target conjugate X1′-SN38 or X2′-SN38 for example, has a cytotoxicity towards cells in a cell culture up to 10-fold or 200-fold or 1000-fold or 10000-fold lower than the active agent SN38 (depending on the cell line used) enabling cell killing with the target conjugates and subsequent visible light irradiation with a wavelength >550 nm.

In another embodiment, the active agent consists of a pharmaceutical agent, a chemotherapeutic agent or small molecule inhibitor and can be photoreleased from the target conjugate using visible light with a wavelength >550 nm in a spatiotemporally highly controlled fashion in a subject in order to treat a highly localized disease, such as cancer using light delivered to the area of the disease via an optical probe/optical fiber setup to illuminate the given location within the said subject. Upon photorelease of the pharmaceutical agent, chemotherapeutic agent or small molecule inhibitor, highly localized effect restricted to the disease site can be achieved. Highly localized increase in the concentration of a said agent can increase the pharmaceutical effect while reducing off-site toxicities and further side-effects.

For example the target conjugate can be the above mentioned SN38 derivative for the treatment of solid tumors. Administering the compound to the subject and subsequent irradiation of the solid tumor can increase the therapeutic index of SN38 via the localized photorelease of the active agent.

In another embodiment, the invention provides a method for treating cancer using the target conjugate, wherein the active agent consists of a pharmaceutical agent, chemotherapeutic agent or small molecule inhibitor that can be photoreleased from the target conjugate using visible light with a wavelength >550 nm in a spatiotemporally highly controlled fashion in a subject with a solid tumor. For example, the target conjugate can be one of the above mentioned SN38 derivatives for the treatment of solid tumors.

### 5. Compositions and Methods of Administration

In one embodiment, the photoactivatable conjugate of the present invention comprises a photocaged active pharmaceutical agent.

Therefore, one aspect of the invention relates to pharmaceutical compositions including an effective amount of a photoactivatable conjugate of the invention and a pharmaceutically acceptable excipient. The compositions are suitable for veterinary or human administration.

Any suitable route of administration may be employed for providing a patient with an effective dosage of the photoactivatable compounds described herein. For example, oral or parenteral administration, and the like may be employed. Dosage forms include tablets, dispersions, suspensions, solutions, capsules, and the like. The compounds are typically administered in admixture with suitable pharmaceutical diluents, excipients, or carriers (collectively referred to herein as “pharmacologically acceptable excipients”) suitably selected with respect to the intended form of administration, and consistent with conventional pharmaceutical practices.

After administration of the photocaged active agent to the subject, the locus of the disorder is irradiated with a wavelength >550 nm so as to release the active agent. An optical probe/optical fiber setup may be used to illuminate a location within the said subject.

Preferably the disorder is highly localizable in the body of the subject.

In one embodiment the photoactivatable conjugate comprises cytotoxic/chemotherapeutic agent or inhibitor used for the destruction of cancer cells, and the disorder is cancer and in particularly, cancer manifesting in solid tumors.

### EXPERIMENTAL

### General Methods

All starting materials were obtained from commercial suppliers and used without further purification. Analytical thin-layer chromatography (TLC) was performed on silica gel 60 F254 precoated aluminum TLC plates from Merck. Flash column chromatography was performed on Teledyne Isco CombiFlash® Rf+ automated flash chromatographer with silica gel (25-40 μm) from Zeochem. NMR spectra were recorded on a Varian Inova 500 MHz spectrometer. Chemical shifts (δ) are given in parts per million (ppm) using solvent signals or TMS as the reference. Coupling constants (J) are reported in Hertz (Hz). Analytical RP-HPLC-UV/Vis-MS measurements were employed using a Shimadzu LCMS-2020 instrument applying a Gemini C18 column (100×2.00 mm I.D.) in which the stationary phase is 5 m silica with a pore size of 110 Å. The chromatograms were detected by a UV-Vis diode array (190-800 nm) and an ESI-MS detector. The following linear gradient elution profile was applied, 0 min 0% B; 6.5 min 100% B; 7 min 0% B; 8 min. 0% B) with eluent A (2% HCOOH, 5% MeCN and 93% water) and B (2% HCOOH, 80% MeCN and 18% water) at a flow rate of 1.0 mL min-1 at 30° C. The samples were dissolved in MeCN—water mixtures. Spectroscopic measurements were performed on a Jasco FP 8300 spectrofluorometer. Quartz cuvettes with path length of 1 cm were used.

### Uncaging Experiments

The uncaging experiments were performed using green (4 W input power, λₘₐₓ=549 nm half-width: 16 nm, output power: 72 mW), orange (4 W input power, λₘₐₓ=605 nm half-width: 10 nm, output power: 140 mW) and red (4 W input power, λₘₐₓ=658 nm half-width: 10 nm, output power: 210 mW) LEDs as light source. Sample solutions were prepared, each containing 1 ml of solvent (90% v/v water-MeCN or 90% PBS-MeCN or 100% methanol for the photochemical quantum yield determination) and 0.1 mM or 0.05 mM concentration of the photoactivatable conjugates. The samples were irradiated for a given time using continuous water cooling for the light source. Then, the samples were transferred to the HPLC-UV-MS system and the chromatograms were recorded. The traces at 195 nm, 254 nm or 369 nm (dependent on the cargo moieties) and at the absorption maxima of the photoremovable protecting groups (530 nm, 570 nm or 640 nm) were compared and the corresponding peaks were integrated and compared to reference calibration sets. The possible photoproducts were identified by their corresponding m/z value and also from the UV/VIS spectra from the DAD-equipped instrument. For the dark stability experiments, the HPLC sample solutions were kept in the dark and chromatograms were recorded multiple times.

### Cell Sample Preparation for Fluorescence Imaging Experiments

SKOV3 (ATCC HTB-77) cells were maintained in McCoy's 5A (Modified) Medium, HEPES (Gibco 22330021) media supplemented with 10% FBS (Gibco 10500-064), 1% penicillin-streptomycin (Gibco 15140-122). The cells were cultured at 37° C. in a 5% CO₂ atmosphere and passaged—using trypsin (Gibco 25300-054)—every 3-4 days up to 20 passages.

### Live Cell Fluorescence Imaging

SKOV3 (4,5000 cell/well) cells were transferred into μ-Slide 8 well plates (Ibidi 80827) and were incubated for 40 h at 37° C. in a 5% CO₂ atmosphere. After 30 min treatment with compounds X1′-SN38 and X2′—SN38 in the concentration of 1 μM and in some cases in combination with 1 nM MitoTracker Deep Red FM (Invitrogen M22426) or 1 nM LysoTracker Deep red (Invitrogen L12492) or 50 nM MitoTracker Red FM (Invitrogen M22425) or 50 nM LysoTracker red DND-99 (Invitrogen L7528). The samples were subjected to microscopy analyses.

Confocal images were acquired on a Leica TCS SP8 STED 3×microscope using 552 nm and 638 nm lasers for excitation. The images were taken using a Leica HC PL APO 100×/1.40 oil immersion objective along with Leica PMT and HyD detectors. All images were taken using dual detection (channel 1: PMT detector, channel 2: HyD detector). The channels were selected for minimal bleed-through between the green/yellow and red channels. The following wavelengths were selected in case of X2′-SN38: green/yellow channel (for the emission of the MitoTracker Red and LysoTracker Red) 564-600 nm using 552 nm excitation; red channel (for the emission of the photoremovable moieties) 715-800 nm using 638 nm excitation. For the trackers, a concentration of 50 nM was used, for compound X2′-SN38, concentrations of 100 nM and 0.1 uM were used. For compound X1′-SN38, MitoTracker Deep Red and LysoTracker Deep Red were used. In this case, the following wavelengths were used: green/yellow channel (for compound 11): 565-624 nm using 552 nm excitation; red channel (for the trackers): 700-800 nm using 638 nm excitation. The images were processed using ImageJ software.

### Cytotoxicity Determination

A viability test was carried out to assess the toxicity of SN38 as well as the photoremovable protecting group-SN38 conjugates X1′-SN38 and X2′-SN38 after light irradiation and in the dark on SKOV3 cells. Cells were transferred into a 48-well plate (Thermo Fisher Scientific, 130187) (4,500 cells/well) and incubated for 20-24 h at 37° C. in a 5% CO₂ atmosphere in McCoy's 5A (Modified) Medium, HEPES (Gibco 22330021) supplemented with 10% FBS (Gibco 10500-064), 1% penicillin-streptomycin (Gibco 15140-122). Cells were treated with compounds in the concentration range of 10⁻¹¹-10⁻⁴ M for 90 min followed by light irradiation or kept in the dark at 37° C. in 5% CO₂ atmosphere. After treatment and irradiation, cells were kept at 37° C. in 5% CO₂ atmosphere for 72 hours. After the incubation period, supernatants were replaced with 0.5 mg/ml MTT (3-(4,5-dimethylthiazol-2-yl)-2,5-diphenyltetrazolium bromide; Sigma M5655-1G) solution (in complete DMEM: Dulbecco's modified Eagle's medium (DMEM, Gibco 41965-039), supplemented with 10% FBS (Gibco 10500-064), 1% penicillin-streptomycin (Gibco 15140-122), 1% Glutamax (Gibco 35050-061) and 1% sodium pyruvate (Life Technologies, Gibco 11360-070)) and incubated for 120 min at 37° C. in the dark. The insoluble formazan crystals were dissolved in 250 μl DMSO. Absorbance was detected at 540 nm using a Biotek Synergy 2 Cytation 3 imaging plate reader with Gen5 software version 3.08 (Biotek Winooski, VT, USA). Viability was expressed as percentage (n=3) of the readings of untreated control cells.

### EXAMPLES

### 1. Synthesis of Compounds X

### 1.1 Synthesis of Compounds X Wherein W═OH and A=CH

### Synthesis of Key Intermediates from Xanthones—Umpolung Strategy

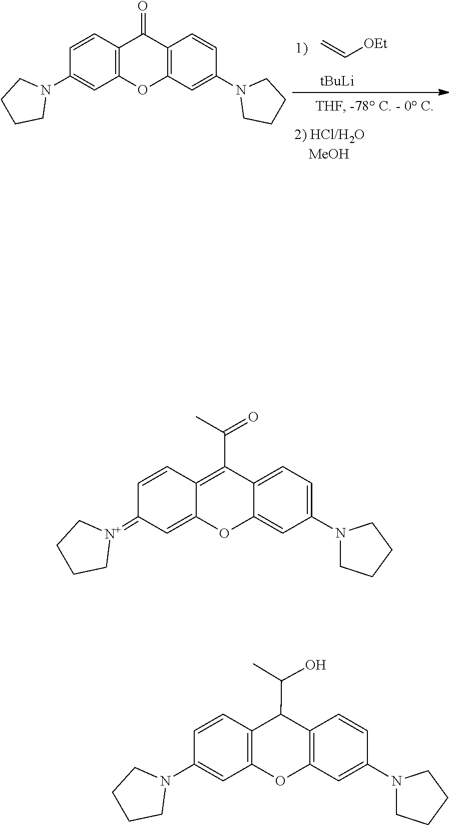

### 1.1.1 Synthesis of 1-(3,6-di(pyrrolidin-1-yl)-9H-xanthen-9-yl)ethan-1-ol (3)

In a previously dried round-bottom flask anhydrous THF and ethyl-vinyl ether (846 uL, 8.97 mmol, 15 equiv.) was cooled to −78° C. under N₂ atmosphere and tert-butyllithium (1.7 M in pentane, 2.46 mL, 4.186 mmol, 7.5 equiv.) was added dropwise. The resulting yellow solution was stirred for 20 min at −78° C., then warmed up to 0° C. in an ice-water bath and kept at this temperature for 5 min. Next, the reaction mixture was cooled back down to −78° C., then the suspension of 3,6-di(pyrrolidin-1-yl)-9H-xanthen-9-one (1) (200 mg, 0.598 mmol, 1 equiv.) in dry THF was added dropwise. The orange solution was stirred for 20 min at −78° C., then the cooling bath was removed, and the reaction mixture was allowed to warm to room temperature and stirred for one more hour. After completion, the reaction was quenched with saturated NH₄Cl (5 mL), then poured into 50 mL methanol. With the dropwise addition of cc. HCl (2.5 mL) the orange solution turned dark pink and was stirred for 16 hours at room temperature (hydrolysis step). The reaction was monitored using LC-MS and after the completion of the hydrolysis, water was added and the mixture was extracted with CH₂Cl₂ 5 times (5×50 mL), the combined organic layers were washed with brine and dried over Mg₂SO₄. After the evaporation of the volatiles, the dark pink solid (compound 2) was used without further purification. Yield: 236 mg, 0.595 mmol, 99%.

¹H NMR (500 MHz, Acetonitrile-d₃; d-TFA) δ 7.47 (d, J=9.3 Hz, 2H), 6.95 (dd, J=9.3, 2.3 Hz, 2H), 6.67 (d, J=2.3 Hz, 2H), 3.61-3.58 (m, 8H), 2.71 (s, 3H), 2.11-2.08 (m, 8H).

¹³C NMR (126 MHz, Acetonitrile-d₃; d-TFA) δ 203.0, 158.5, 158.2, 156.0, 130.5, 116.9, 114.7, 110.0, 98.2, 50.2, 25.8.

HRMS: [M]⁺: calcd for [C₂₃H₂₅N₂O₂]⁺: 361.1910, found: 361.1906.

The crude acetyl-pyronine compound (2) (236 mg, 0.595 mmol, 1 equiv.) was dissolved in ethanol (30 mL) and NaBH₄ (225 mg, 5.95 mmol, 10 equiv.) was added to the solution. The reaction was stirred for one hour at room temperature until the LC-MS indicated complete conversion. 20 mL water was added to the mixture at 0° C. and it was subsequently extracted with CH₂Cl₂ 3 times (3×50 mL). The combined organic layers were washed with water, brine and dried over Mg₂SO₄. After filtration and evaporation, the pink solid (compound 3) was used without any further purification. Yield: 200 mg, 0.549 mmol, 92%.

¹H NMR (500 MHz, Chloroform-d) δ 7.15-7.02 (m, 2H), 6.36-6.28 (m, 4H), 3.84-3.73 (m, 2H), 3.27-3.21 (m, 8H), 2.01-1.95 (m, 8H), 0.97 (d, J=5.9 Hz, 3H).

¹³C NMR (126 MHz, Chloroform-d) δ 154.1, 153.8, 148.3, 148.2, 129.9, 129.7, 110.1, 109.2, 107.2, 107.1, 99.4, 99.2, 73.8, 47.9, 45.5, 25.6, 18.8.

HRMS: [M+H]⁺: calcd for [C₂₃H₂₉N₂O₂]⁺: 365.2223, found: 365.2217.

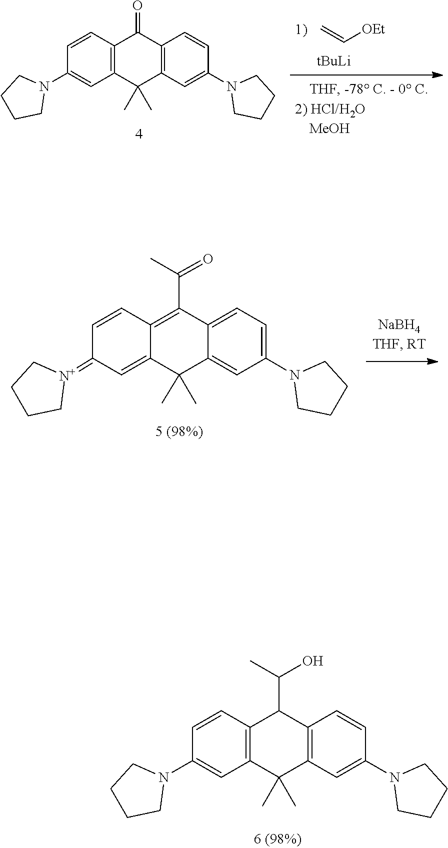

### 1.1.2 Synthesis of 1-(10,10-dimethyl-3,6-di(pyrrolidin-1-yl)-9,10-dihydroanthracen-9-yl)ethan-1-ol (6)

In a previously dried round-bottom flask anhydrous THF and ethyl-vinyl ether (785 uL, 8.32 mmol, 15 equiv.) was cooled to −78° C. under N₂ atmosphere and tert-butyllithium (1.7 M in pentane, 2.45 mL, 4.16 mmol, 7.5 equiv.) was added dropwise. The resulting yellow solution was stirred for 20 min at −78° C., then warmed up to 0° C. in an ice-water bath and kept at this temperature for 5 min. Next, the reaction mixture was cooled back down to −78° C., the suspension of 10,10-dimethyl-3,6-di(pyrrolidin-1-yl)anthracen-9 (10H)-one (4) (200 mg, 0.55 mmol, 1 equiv.) in dry THF was added dropwise. The orange solution was stirred for 20 min at −78° C., then the cooling bath was removed, and the reaction mixture was allowed to warm to room temperature and stirred for one more hour. After completion, the reaction was quenched with saturated NH₄Cl (5 mL), then poured into 50 mL methanol. With the dropwise addition of cc. HCl (2.5 mL) the orange solution turned dark blue and was stirred for 16 hours at room temperature (hydrolysis step). The reaction was monitored using LC-MS and after the completion of the hydrolysis, water was added and the mixture was extracted with CH₂Cl₂ 5 times (5×100 mL), the combined organic layers were washed with brine and dried over Mg₂SO₄. After the evaporation of the volatiles, the dark blue solid (5) was used without further purification. Yield: 228 mg, 0.539 mmol, 98%.

¹H NMR (500 MHz, Acetonitrile-d₃; d-TFA) δ 7.38 (d, J=9.2 Hz, 2H), 7.03 (d, J=2.4 Hz, 2H), 6.78 (dd, J=9.2, 2.4 Hz, 2H), 3.68-3.63 (m, 8H), 2.67 (s, 3H), 2.11-2.07 (m, 8H), 1.70 (s, 6H).

¹³C NMR (126 MHz, Acetonitrile-d₃; d-TFA) δ 206.3, 161.6, 158.7, 158.4, 158.1, 155.0, 135.7, 115.5, 113.9, 50.2, 42.7, 33.6, 25.8.

HRMS: [M]⁺: calcd for [C₂₆H31N₂O]⁺: 387.2430, found: 387.2421.

The crude acetyl-pyronine compound (5) (228 mg, 0.539 mmol, 1 equiv.) was dissolved in ethanol (10 mL) and NaBH₄ (204 mg, 5.539 mmol, 10 equiv.) was added to the solution. The reaction was stirred for one hour at room temperature until the LC-MS indicated complete conversion. 10 mL water was added to the mixture at 0° C. and it was subsequently extracted with CH₂Cl₂ 5 times (5×50 mL). The combined organic layers were washed with water, brine and dried over Mg₂SO₄. After filtration and evaporation, the blue solid (compound 6) was used without any further purification.

Yield: 211 mg, 0.541 mmol, 98%.

¹H NMR (500 MHz, Acetonitrile-d₃) δ 7.23 (d, J=8.4 Hz, 1H), 7.14 (d, J=8.3 Hz, 1H), 6.77 (dd, J=5.4, 2.5 Hz, 2H), 6.55-6.50 (m, 2H), 3.85-3.77 (m, 2H), 3.35-3.32 (m, 8H), 2.19 (s, 1H), 2.06-2.02 (m, 8H), 1.71 (d, J=7.2 Hz, 6H), 0.84 (d, J=6.1 Hz, 3H).

¹³C NMR (126 MHz, Acetonitrile-d₃) δ 148.19, 148.18, 146.5, 146.3, 131.0, 130.8, 123.1, 122.8, 111.03, 110.99, 110.5, 110.2, 75.2, 50.7, 48.6, 48.5, 39.7, 35.4, 33.8, 26.1, 20.3.

HRMS: [M+H]⁺: calcd for [C₂₆H₃₅N₂O]⁺: 391.2743, found: 391.2744.

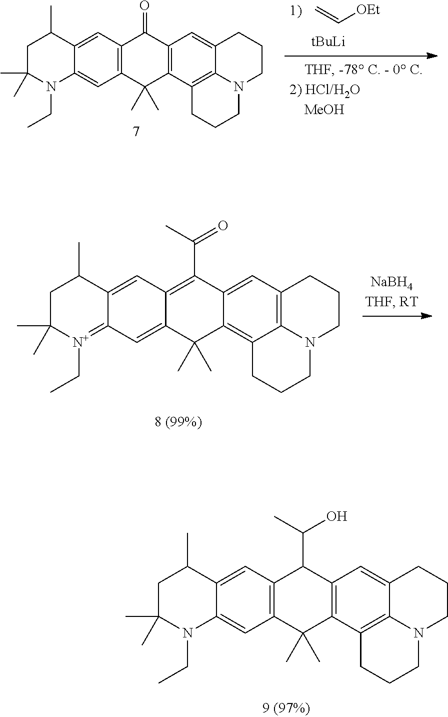

### 1.1.3 Synthesis of the Extended Carbopyronin Frame (9)

In a previously dried round-bottom flask anhydrous THF (5 mL) and ethyl-vinyl ether (320 μL, 3.39 mmol, 15 equiv.) was cooled to −78° C. under N₂ atmosphere and tert-butyllithium (1.7 M in pentane, 997 μL, 1.69 mmol, 7.5 equiv.) was added dropwise. The resulting yellow solution was stirred for 20 min at −78° C., then warmed up to 0° C. in an ice-water bath and kept at this temperature for 5 min. Next, the reaction mixture was cooled back down to −78° C., the suspension of the xanthone 7 (100 mg, 0.226 mmol, 1 equiv.) in dry THF (5 mL) was added dropwise. The orange solution was stirred for 20 min at −78° C., then the cooling bath was removed, and the reaction mixture was allowed to warm to room temperature and stirred for one more hour. After completion, the reaction was quenched with saturated NH₄Cl (20 mL), then poured into 15 mL methanol. With the dropwise addition of cc. HCl (2 mL) the orange solution turned dark blue and was stirred for 24 hours at room temperature (hydrolysis step). The reaction was monitored using LC-MS and after the completion of the hydrolysis, water was added and the mixture was extracted with CH₂Cl₂ 5 times (5×100 mL), the combined organic layers were washed with brine and dried over Mg₂SO₄. After the evaporation of the volatiles, the dark blue solid was used without further purification.

Yield: 113 mg, 0.224 mmol, 99%.

¹H NMR (500 MHz, Chloroform-d) δ 6.93 (s, 1H), 6.87 (s, 1H), 6.84 (s, 1H), 3.77-3.55 (m, 6H), 3.26-3.09 (m, 2H), 2.87-2.73 (m, 3H), 2.68 (s, 3H), 2.14-1.99 (m, 2H), 1.88-1.77 (m, 10H), 1.48 (s, 3H), 1.38-1.34 (m, 6H), 1.32 (d, J=6.5 Hz, 3H).

MS: [M]⁺: calcd for [C₃₂H₄₁N₂O]⁺: 469, found: 469.

The acetyl-pyronine compound 8 (113 mg, 0.224 mmol, 1 equiv.) was dissolved in ethanol (30 mL) and NaBH₄ (85 mg, 2.24 mmol, 10 equiv.) was added to the solution. The reaction was stirred for one hour at room temperature until the LC-MS indicated complete conversion. 20 mL water was added to the mixture at 0° C. and it was subsequently extracted with CH₂Cl₂ 5 times (5×100 mL). The combined organic layers were washed with water, brine and dried over Mg₂SO₄. After filtration and evaporation, the blue solid (compound 9) was used without any further purification.

Yield: 104 mg, 0.220 mmol, 97%.

¹H NMR (500 MHz, Chloroform-d) δ 6.99-6.91 (m, 1H), 6.75-6.69 (m, 1H), 6.65-6.60 (m, 1H), 3.73-3.63 (m, 2H), 3.52-3.45 (m, 1H), 3.21-3.13 (m, 4H), 3.06-2.85 (m, 4H), 2.79-2.68 (m, 2H), 1.98-1.92 (m, 4H), 1.86-1.82 (m, 3H), 1.78-1.74 (m, 3H), 1.74-1.67 (m, 1H), 1.64-1.54 (m, 1H), 1.39-1.20 (m, 12H), 1.00-0.92 (m, 3H).

MS: [M]⁺: calcd for [C₃₂H₄₅N₂O]⁻: 473, found: 473.

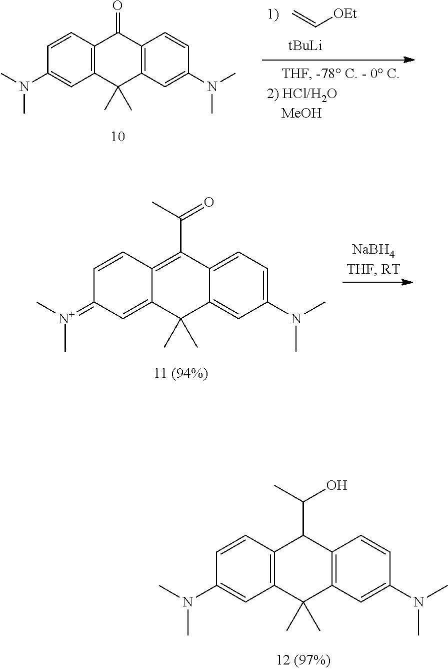

### 1.1.4 Synthesis of 1-(3,6-Bis(dimethylamino)-10,10-dimethyl-9,10-dihydroanthracen-9-yl)ethan-1-ol (12)

In a previously dried round-bottom flask anhydrous THF (30 mL) and ethyl-vinyl ether (4.59 mL, 48.63 mmol, 15 equiv.) was cooled to −78° C. under N₂ atmosphere and tert-butyllithium (1.7 M in pentane, 14.3 mL, 24.32 mmol, 7.5 equiv.) was added dropwise. The resulting yellow solution was stirred for 20 min at −78° C., then warmed up to 0° C. in an ice-water bath and kept at this temperature for 5 min. Next, the reaction mixture was cooled back down to −78° C., the suspension of 3,6-bis(dimethylamino)-10,10-dimethylanthracen-9 (10H)-one (10) (1.00 g, 3.24 mmol, 1 equiv.) in dry THF (15 mL) was added dropwise. The orange solution was stirred for 20 min at −78° C., then the cooling bath was removed, and the reaction mixture was allowed to warm to room temperature and stirred for one more hour. After completion, the reaction was quenched with saturated NH₄Cl (20 mL), then poured into 100 mL methanol. With the dropwise addition of cc. HCl (15 mL) the orange solution turned dark blue and was stirred for 1 hours at room temperature (hydrolysis step). The reaction was monitored using LC-MS and after the completion of the hydrolysis, water was added and the mixture was extracted with CH₂Cl₂ 5 times (5×100 mL), the combined organic layers were washed with brine and dried over Mg₂SO₄. After the evaporation of the volatiles, the dark blue solid (compound 11) was used without further purification.

Yield: 1.133 g, 3.06 mmol, 94%.

¹H NMR (500 MHz, Acetonitrile-d₃) δ 7.40 (d, J=9.4 Hz, 2H), 7.15 (d, J=2.6 Hz, 2H), 6.91 (dd, J=9.3, 2.6 Hz, 2H), 3.32 (s, 12H), 2.67 (s, 3H), 1.71 (s, 6H).

¹³C NMR (126 MHz, Acetonitrile-d₃) δ 206.1, 162.0, 158.3, 157.7, 135.8, 116.0, 114.7, 113.1, 42.9, 41.6, 33.6, 30.7.

MS: [M]⁺: calcd for [C₂₂H₂₇N₂O]⁺: 335, found: 335.

The crude acetyl-pyronine compound 11 (1.133 g, 3.06 mmol, 1 equiv.) was dissolved in ethanol (100 mL) and NaBH₄ (1.156 g, 30.55 mmol, 10 equiv.) was added to the solution. The reaction was stirred for one hour at room temperature until the LC-MS indicated complete conversion. 50 mL water was added to the mixture at 0° C. and it was subsequently extracted with CH₂Cl₂ 5 times (5×100 mL). The combined organic layers were washed with water, brine and dried over Mg₂SO₄. After filtration and evaporation, the blue solid (compound 12) was used without any further purification.

Yield: 1.003 g, 2.96 mmol, 97%.

¹H NMR (500 MHz, Chloroform-d) δ 7.20 (d, J=8.5 Hz, 1H), 7.17 (d, J=8.4 Hz, 1H), 6.94 (d, J=2.6 Hz, 1H), 6.92 (d, J=2.7 Hz, 1H), 6.69 (m, 2H), 3.83 (s, 1H), 3.76 (m1H), 2.98 (s, 12H), 1.74 (s, 3H), 1.67 (s, 3H), 1.03 (d, J=6.2 Hz, 3H).

¹³C NMR (126 MHz, Chloroform-d) δ 149.9, 149.8, 145.5, 145.5, 129.8, 129.6, 123.5, 123.4, 111.4, 111.3, 111.3, 110.9, 74.3, 50.7, 41.1, 39.4, 35.8, 32.9, 20.4.

MS: [M+H]⁺: calcd for [C₂₂H₃₁N₂O]⁺: 339, found: 339.

### Synthesis of Key Intermediates from Xanthones—Hydroboration-Oxidation Strategy

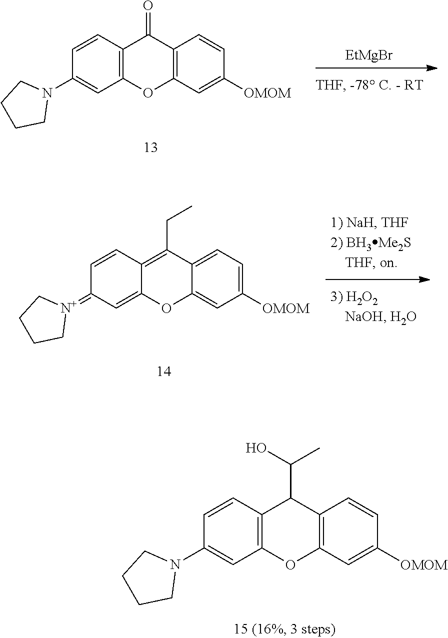

### 1.1.5 Synthesis of 1-(3-(methoxymethoxy)-6-(pyrrolidin-1-yl)-9H-xanthen-9-yl)ethan-1-ol (15)

Grignard reaction (step 1): in a dried round-bottom flask 3-(methoxymethoxy)-6-(pyrrolidin-1-yl)-9H-xanthen-9-one (13) (150 mg, 0.46 mmol, 1 equiv.) in anhydrous THF (5 mL) was cooled to −78° C. under N₂ atmosphere and EtMgBr (1 M in THF, 4.6 mL, 4.6 mmol, 10 equiv.) was added dropwise. After stirring for 30 min, the cooling bath was removed, and the reaction was stirred for one more hour at room temperature. Then, saturated NH₄Cl (5 mL) was added dropwise and the mixture was extracted with EtOAc 3 times (3×50 mL). The collected organic phases were washed with brine and dried over Mg₂SO₄. After the evaporation, the dark orange solid (compound 14) was used immediately without purification. Crude NMR, mixture of the E/Z isomers of the exo-form (double bond located outside the ring):

¹H NMR (500 MHz, Acetonitrile-d₃) δ 7.60 (d, J=8.7 Hz, 1H), 7.57 (d, J=8.7 Hz, 1H), 6.87 (dd, J=8.6, 2.5 Hz, 1H), 6.84 (d, J=2.5 Hz, 1H), 6.47 (dd, J=8.7, 2.5 Hz, 1H), 6.34 (d, J=2.5 Hz, 1H), 5.92 (q, J=7.4 Hz, 1H), 5.29 (s, 2H), 3.56 (s, 3H), 3.38-3.30 (m, 4H), 2.16 (d, J=7.5 Hz, 3H), 2.10-2.04 (m, 4H).

¹H NMR (500 MHz, Acetonitrile-d₃) δ 7.71 (d, J=8.6 Hz, 1H), 7.44 (d, J=8.7 Hz, 1H), 6.92 (dd, J=8.6, 2.6 Hz, 1H), 6.90 (d, J=2.5 Hz, 1H), 6.44 (dd, J=8.7, 2.5 Hz, 1H), 6.28 (d, J=2.5 Hz, 1H), 5.92 (q, J=7.4 Hz, 1H), 5.32 (s, 2H), 3.57 (s, 3H), 3.38-3.30 (m, 4H), 2.16 (d, J=7.5 Hz, 3H), 2.10-2.04 (m, 4H).

¹³C NMR (126 MHz, Acetonitrile-d₃) δ 158.14, 158.13, 154.6, 154.5, 152.8, 152.7, 149.41, 149.37, 129.8, 129.7, 127.1, 127.0, 126.4, 125.2, 124.9, 120.7, 114.4, 113.7, 113.1, 111.8, 110.9, 109.6, 108.2, 104.8, 104.6, 99.3, 99.0, 95.4, 95.3, 56.5, 56.4, 48.4, 48.4, 30.7, 26.09, 26.07, 16.2, 16.1.

HRMS: [M]⁺: calcd for [C₂₁H₂₄NO₃]⁺: 338.1750, found: 338.1753.

Hydroboration-oxidation (steps 2 & 3): the resulting ethyl-pyronine (compound 14) was dissolved in anhydrous THF (5 mL) and NaH (74 mg, 1.84 mmol, 4 equiv.) was added to the solution under N₂ atmosphere. After 15 min, the yellow solution was filtered through a syringe filter into a deoxygenated round-bottom flask. The solution was cooled to 0° C. and borane-dimethyl sulfide comlex (426 uL, 4.6 mmol, 10 equiv.) was added dropwise. The reaction mixture was stirred overnight at room temperature. Then, the mixture was poured into methanol (15 mL) and 3 M NaOH (3 mL) and 30% H₂O₂ (3 mL) were added dropwise. After stirring for 30 min, the mixture was extracted with EtOAc 3 times (3×50 mL) and the combined organic phases were washed with Na₂S₂O₃ and brine, dried over Mg₂SO₄ and the solvents were evaporated. The crude product was purified by RP flash chromatography (eluent:water:MeCN 5% to 100%) to afford 15 as a colorless solid.

Yield for the 3 steps: 26 mg, 0.073 mmol, 16%.

Crude NMR, mixture of two diastereomers:

¹H NMR (500 MHz, Acetonitrile-d₃) δ 7.25-7.14 (m, 1H), 7.13-7.03 (m, 1H), 6.78-6.69 (m, 2H), 6.37-6.32 (m, 1H), 6.23 (d, J=2.4 Hz, 1H), 5.17 (t, J=2.4 Hz, 2H), 3.85-3.73 (m, 2H), 3.45-3.40 (m, 3H), 3.27-3.21 (m, 4H), 2.01-1.96 (m, 4H), 0.84-0.77 (m, 3H).

¹³C NMR (126 MHz, Acetonitrile-d₃) δ 157.9, 157.8, 154.6, 154.4, 154.4, 154.1, 149.3, 149.3, 131.8, 131.4, 131.3, 130.9, 117.9, 117.2, 112.0, 111.9, 110.1, 109.7, 108.4, 104.8, 104.5, 99.5, 99.2, 95.5, 74.1, 74.1, 56.4, 48.5, 45.9, 45.7, 26.1, 18.9, 18.7.

HRMS: [M+H]⁺: calcd for [C₂₁H₂₆NO₄]⁺: 356.1856, found: 356.1850.

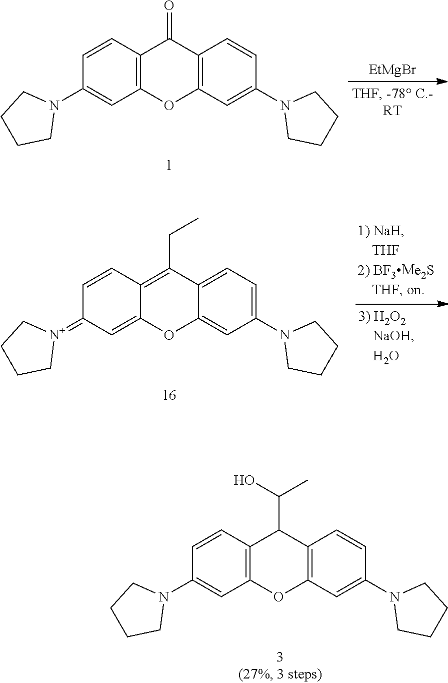 [SMILES: "EtMgBr" ; O=c1c2ccc(N3CCCC3)cc2oc2cc(N3CCCC3)ccc12 ; CCc1c2ccc(=[N+]3CCCC3)cc-2oc2cc(N3CCCC3)ccc12 ; CC(O)C1c2ccc(N3CCCC3)cc2Oc2cc(N3CCCC3)ccc21]

### 1.1.6 Synthesis of 1-(3,6-di(pyrrolidin-1-yl)-9H-xanthen-9-yl)ethan-1-ol (3, alternative route)

Grignard reaction (step 1): in a dried round-bottom flask 3,6-di(pyrrolidin-1-yl)-9H-xanthen-9-one (1) (300 mg, 0.897 mmol, 1 equiv.) in anhydrous THF (5 mL) was cooled to −78° C. under N₂ atmosphere and EtMgBr (1 M in THF, 8.97 mL, 8.97 mmol, 10 equiv.) was added dropwise. After stirring for 30 min, the cooling bath was removed, and the reaction was stirred for one more hour at room temperature. Then, saturated NH₄Cl (5 mL) was added dropwise and the mixture was extracted with EtOAc 3 times (3×50 mL). The collected organic phases were washed with brine and dried over Mg₂SO₄. After the evaporation, the dark pink solid (compound 16) was used immediately without purification.

Crude NMR; dye-form (double bond located in the ring):

¹H NMR (500 MHz, Acetonitrile-d₃; d-TFA) δ 7.90 (d, J=9.4 Hz, 2H), 6.88 (dd, J=9.4, 2.4 Hz, 2H), 6.44 (d, J=2.3 Hz, 2H), 3.53-3.49 (m, 8H), 3.25 (q, J=7.7 Hz, 2H), 2.10-2.06 (m, 8H), 1.32 (t, J=7.7 Hz, 3H).

¹³C NMR (126 MHz, Acetonitrile-d₃; d-TFA) δ 163.6, 158.2, 155.5, 130.2, 115.9, 113.4, 97.3, 49.8, 25.9, 22.2, 16.1.

HRMS: [M]⁺: calcd for [C₂₃H₂₇N₂O]: 347.2117, found: 347.2112.

Hydroboration-oxidation (steps 2 & 3): the resulting ethyl-pyronine (compound 16) was dissolved in anhydrous THF (5 mL) and NaH (86 mg, 3.58 mmol, 4 equiv.) was added to the solution under N₂ atmosphere. After 15 min, the yellow solution was filtered through a syringe filter into a deoxygenated round-bottom flask. The solution was cooled to 0° C. and borane-dimethyl sulfide comlex (850 uL, 8.97 mmol, 10 equiv.) was added dropwise. The reaction mixture was stirred overnight at room temperature. Then, the mixture was poured into methanol (20 mL) and 3 M NaOH (5 mL) and 30% H₂O₂ (5 mL) were added dropwise. After stirring for 30 min, the mixture was extracted with EtOAc 3 times (3×50 mL) and the combined organic phases were washed with Na₂S₂O₃ and brine, dried over Mg₂SO₄ and the solvents were evaporated to afford 3 as a colorless solid.

Yield for the 3 steps: 87 mg, 0.239 mmol, 27%.

¹H NMR (500 MHz, Chloroform-d) δ 7.15-7.02 (m, 2H), 6.36-6.28 (m, 4H), 3.84-3.73 (m, 2H), 3.27-3.21 (m, 8H), 2.01-1.95 (m, 8H), 0.97 (d, J=5.9 Hz, 3H).

¹³C NMR (126 MHz, Chloroform-d) δ 154.1, 153.8, 148.3, 148.2, 129.9, 129.7, 110.1, 109.2, 107.2, 107.1, 99.4, 99.2, 73.8, 47.9, 45.5, 25.6, 18.8.

HRMS: [M+H]⁺: calcd for [C₂₃H₂₉N₂O₂]⁺: 365.2223, found: 365.2217.

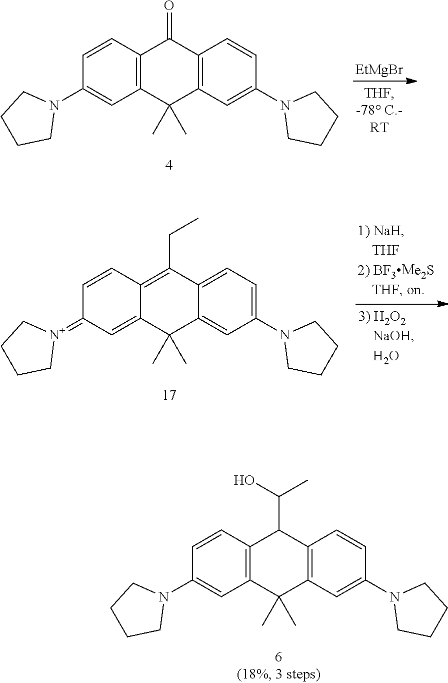 [SMILES: "EtMgBr" ; CC1(C)c2cc(N3CCCC3)ccc2C(=O)c2ccc(N3CCCC3)cc21 ; CCC1=C2C=CC(=[N+]3CCCC3)C=C2C(C)(C)c2cc(N3CCCC3)ccc21 ; CC(O)C1c2ccc(N3CCCC3)cc2C(C)(C)c2cc(N3CCCC3)ccc21]

### 1.1.7 Synthesis of 1-(10,10-dimethyl-3,6-di(pyrrolidin-1-yl)-9,10-dihydroanthracen-9-yl)ethan-1-ol (6, alternative route)

Grignard reaction (step 1): in a dried round-bottom flask 10,10-dimethyl-3,6-di(pyrrolidin-1-yl)anthracen-9 (10H)-one (4) (50 mg, 0.139 mmol, 1 equiv.) in anhydrous THF (5 mL) was cooled to −78° C. under N₂ atmosphere and EtMgBr (1 M in THF, 1.39 mL, 1.390 mmol, 10 equiv.) was added dropwise. After stirring for 30 min, the cooling bath was removed, and the reaction was stirred for one more hour at room temperature. Then, saturated NH₄Cl (5 mL) was added dropwise and the mixture was extracted with EtOAc 3 times (3×50 mL). The collected organic phases were washed with brine and dried over Mg₂SO₄. After the evaporation, the dark blue solid (compound 17) was used immediately without purification.

Hydroboration-oxidation (steps 2 & 3): the resulting ethyl-pyronine (compound 17) was dissolved in anhydrous THF (5 mL) and NaH (16 mg, 0.417 mmol, 4 equiv.) was added to the solution under N₂ atmosphere. After 15 min, the yellow solution was filtered through a syringe filter into a deoxygenated round-bottom flask. The solution was cooled to 0° C. and borane-dimethyl sulfide comlex (132 uL, 1.39 mmol, 10 equiv.) was added dropwise. The reaction mixture was stirred overnight at room temperature. Then, the mixture was poured into methanol (15 mL) and 3 M NaOH (3 mL) and 30% H₂O₂ (3 mL) were added dropwise. After stirring for 30 min, the mixture was extracted with EtOAc 3 times (3×50 mL) and the combined organic phases were washed with Na₂S₂O₃ and brine, dried over Mg₂SO₄ and the solvents were evaporated. The crude product was purified by flash chromatography (eluent:hexane:EtOAc: 0% to 30%) to afford 6 as a colorless solid.

Yield for the 3 steps: 10 mg, 0.026 mmol, 18%.

Crude NMR, two form present in 1:1 ratio (dye- and exo-form):

### Dye-Form:

¹H NMR (500 MHz, Acetonitrile-d₃; d-TFA) δ 8.06 (d, J=9.4 Hz, 2H), 6.95 (d, J=2.5 Hz, 2H), 6.80 (dd, J=9.4, 2.5 Hz, 2H), 3.64-3.60 (m, 8H), 3.30 (q, J=7.7 Hz, 2H), 2.10-2.05 (m, 8H), 1.58 (s, 6H), 1.35 (t, J=7.7 Hz, 3H).

### Exo-Form:

¹H NMR (500 MHz, Acetonitrile-d₃; d-TFA) δ 7.79 (s, 1H), 7.76 (s, 1H), 7.66 (d, J=8.2 Hz, 1H), 7.62 (d, J=8.3 Hz, 1H), 7.51 (s, 1H), 7.50 (s, 1H), 6.37 (q, J=7.3 Hz, 1H), 3.77-3.71 (m, 8H), 2.29-2.22 (m, 8H), 2.10 (d, J=7.4 Hz, 3H), 1.66 (s, 6H).

¹³C NMR (126 MHz, Acetonitrile-d₃; d-TFA) δ 170.2, 158.0, 154.7, 135.4, 130.4, 126.8, 126.4, 120.1, 114.8, 112.4, 68.4, 59.2, 49.8, 42.2, 34.6, 30.7, 28.1, 26.3, 25.9, 24.8, 24.7, 23.7, 17.1, 16.2.

HRMS: [M]⁺: calcd for [C₂₆H₃₃N₂]⁺: 373.2638, found: 373.2621.

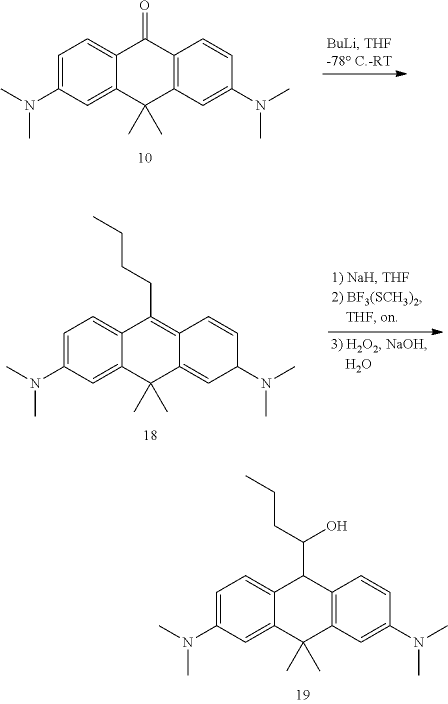

### 1.1.8 Synthesis of 1-(3,6-bis(dimethylamino)-10,10-dimethyl-9,10-dihydroanthracen-9-yl)butan-1-ol (19, alternative route)

Grignard reaction (step 1): in a dried round-bottom flask 3,6-bis(dimethylamino)-10,10-dimethylanthracen-9 (10H)-one (10) (154 mg, 0.50 mmol, 1 equiv.) in anhydrous THF (5 mL) was cooled to −78° C. under N₂ atmosphere and butyl lithium (2.5 M in Hexane, 1.20 mL, 3.0 mmol, 6 equiv.) was added dropwise. After stirring for 30 min, the cooling bath was removed, and the reaction was stirred for one more hour at room temperature. Then, saturated NH₄Cl (5 mL) was added dropwise and the mixture was extracted with EtOAc 3 times (3×50 mL). The collected organic phases were washed with brine and dried over Mg₂SO₄. After the evaporation, the dark blue solid (compound 18) was used immediately without purification.

Hydroboration-oxidation (steps 2): the resulting butyl-pyronine (compound 18) was dissolved in anhydrous THF (5 mL). The solution was cooled to 0° C. and borane-dimethyl sulfide comlex (300 uL, 3.0 mmol, 6 equiv.) was added dropwise. The reaction mixture was stirred overnight at room temperature. Then, the mixture was poured into methanol (15 mL) and 3 M NaOH (3 mL) and 30% H₂O₂ (3 mL) were added dropwise. After stirring for 30 min, the mixture was extracted with EtOAc 3 times (3×50 mL) and the combined organic phases were washed with Na₂S₂O₃ and brine, dried over Mg₂SO₄ and the solvents were evaporated. The crude product was purified by flash chromatography (eluent:hexane:EtOAc: 0% to 30%) to afford 19 as a colorless solid.

Yield for the 2 steps: 32 mg, 0.094 mmol, 17%.

¹H NMR (500 MHz, Chloroform-d) δ 7.19 (d, J=8.4 Hz, 1H), 7.15 (d, J=8.4 Hz, 1H), 6.94 (dd, J=11.1, 2.6 Hz, 2H), 6.69 (dt, J=8.4, 2.7 Hz, 2H), 3.89 (d, J=5.5 Hz, 1H), 3.60 (m, 1H), 2.98 (s, 12H), 2.05 (s, 1H), 1.71 (d, J=21.7 Hz, 6H), 1.47-1.39 (m, 2H), 1.23-1.16 (m, 2H), 0.83 (t, J=7.1 Hz, 3H).

MS: [M+H]⁺: calcd for [C₂₁H₂₉N₂O₂]⁺: 341, found: 341.

### 1.2 Synthesis of Compounds X Wherein W═OH and A=C⁺

### Oxidation of Compounds X Wherein A=CH

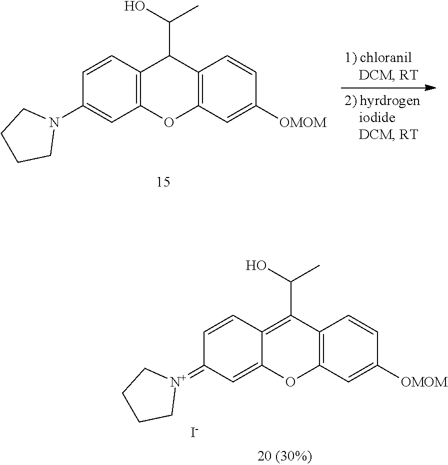

### 1.2.1 Synthesis of 1-(9-(1-hydroxyethyl)-6-(methoxymethoxy)-3H-xanthen-3-ylidene)pyrrolidin-1-ium iodide (20)

1-(3-(methoxymethoxy)-6-(pyrrolidin-1-yl)-9H-xanthen-9-yl)ethan-1-ol (15) (15 mg, 0.042 mmol, 1 equiv.) was dissolved in CH₂Cl₂— MeOH 1:1 (2 mL) and p-chloranil (22 mg, 0.084 mmol, 2 equiv.) was added to the solution. The mixture was stirred until the LC-MS indicated full conversion. The reaction was evaporated and applied directly to a silica gel column and purified by flash chromatography on silica (Eluent: CH₂Cl₂— MeOH 0% to 20%).

The product was redissolved in CH₂Cl₂, one drop of HI (57% in water) was added, stirred for 5 min and another flash chromatography (on silica) was carried out, obtaining the final compound with I-counterion. (eluent: CH₂Cl₂— MeOH 0% to 20%).

Yield: 6 mg, 0.012 mmol, 30%.

MS: [M]⁺: calcd for [C₂₁H₂₄NO₄]⁺: 354, found: 354.

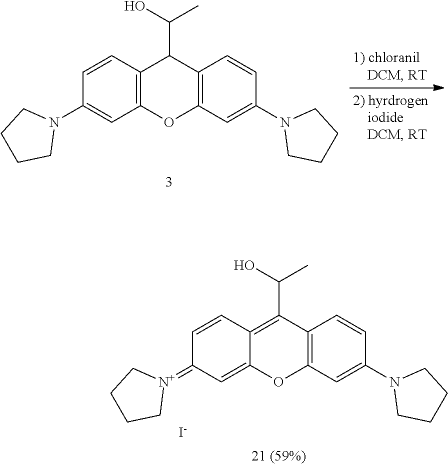 [SMILES: CC(O)C1c2ccc(N3CCCC3)cc2Oc2cc(N3CCCC3)ccc21 ; CC(O)c1c2ccc(=[N+]3CCCC3)cc-2oc2cc(N3CCCC3)ccc12 ; [I-]]

### 1.2.2 Synthesis of 1-(9-(1-hydroxyethyl)-6-(pyrrolidin-1-yl)-3H-xanthen-3-ylidene)pyrrolidin-1-ium iodide (21)

1-(3,6-Di(pyrrolidin-1-yl)-9H-xanthen-9-yl)ethan-1-ol (3) (15 mg, 0.041 mmol, 1 equiv.) was dissolved in CH₂Cl₂— MeOH 1:1 (2 mL) and p-chloranil (21 mg, 0.082 mmol, 2 equiv.) was added to the solution. The mixture was stirred until the LC-MS indicated full conversion. The reaction was evaporated and applied directly to a silica gel column and purified by flash chromatography on silica (Eluent: CH₂Cl₂— MeOH 0% to 20%).

The product was redissolved in CH₂Cl₂, one drop of HI (57% in water) was added, stirred for 5 min and another flash chromatography (on silica) was carried out, obtaining the final compound with I-counterion (eluent: CH₂Cl₂— MeOH 0% to 20%).

Yield: 12 mg, 0.025 mmol, 59%.

¹H NMR (500 MHz, Acetonitrile-d₃) δ 8.46 (d, J=9.6 Hz, 2H), 6.87 (dd, J=9.6, 2.5 Hz, 2H), 6.49 (d, J=2.4 Hz, 2H), 5.94 (q, J=6.9 Hz, 1H), 2.20 (s, 16H), 1.65 (d, J=6.9 Hz, 3H).

¹³C NMR (126 MHz, Acetonitrile-d₃) δ 163.3, 157.5, 154.3, 130.5, 129.5, 114.4, 111.6, 96.4, 65.2, 48.7, 24.3.

MS: [M]⁺: calcd for [C₂₃H₂₇N₂O₂]⁺: 363, found: 363.

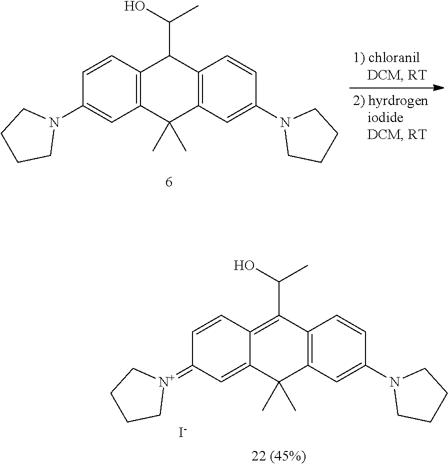 [SMILES: CC(O)C1c2ccc(N3CCCC3)cc2C(C)(C)c2cc(N3CCCC3)ccc21 ; CC(O)C1=C2C=CC(=[N+]3CCCC3)C=C2C(C)(C)c2cc(N3CCCC3)ccc21 ; [I-]]

### 1.2.3 Synthesis of 1-(10-(1-hydroxyethyl)-9,9-dimethyl-7-(pyrrolidin-1-yl)anthracen-2 (9H)-ylidene)pyrrolidin-1-ium iodide (22)

1-(10,10-Dimethyl-3,6-di(pyrrolidin-1-yl)-9,10-dihydroanthracen-9-yl)ethan-1-ol (6) (20 mg, 0.051 mmol, 1 equiv.) was dissolved in CH₂Cl₂— MeOH 1:1 (2 mL) and p-chloranil (26 mg, 0.102 mmol, 2 equiv.) was added to the solution. The mixture was stirred until the LC-MS indicated full conversion. The reaction was evaporated and applied directly to a silica gel column and purified by flash chromatography on silica (Eluent: CH₂Cl₂— MeOH 0% to 20%).

The product was redissolved in CH₂Cl₂, one drop of HI (57% in water) was added, stirred for 5 min and another flash chromatography (on silica) was carried out, obtaining the final compound with I-counterion (eluent: CH₂Cl₂— MeOH 0% to 20%).

Yield: 12 mg, 0.023 mmol, 45%.

¹H NMR (500 MHz, Acetonitrile-d₃) δ 8.16 (d, J=2.3 Hz, 2H), 7.65 (dd, J=8.4, 2.4 Hz, 2H), 7.55 (d, J=8.4 Hz, 2H), 3.66 (d, J=3.9 Hz, 1H), 2.35-2.29 (m, 8H), 2.11 (s, 3H), 1.82 (s, 3H), 1.53 (s, 3H).

¹³C NMR (126 MHz, Acetonitrile-d₃) δ 157.4, 145.9, 139.9, 133.5, 131.0, 120.4, 119.68, 58.7, 58.3, 39.8, 33.8, 27.7, 23.7.

MS: [M]⁺: calcd for [C₂₆H₃₃N₂O]⁺: 389, found: 389.

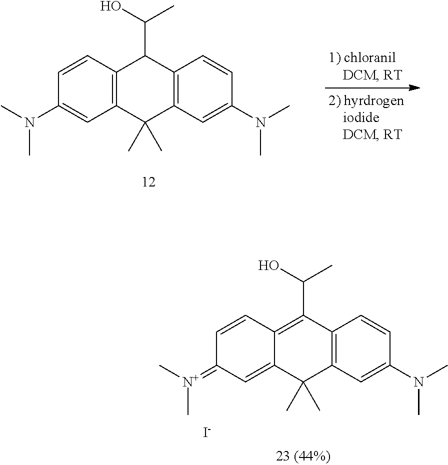

### 1.2.4 Synthesis of N-(7-(Dimethylamino)-10-(1-hydroxyethyl)-9,9-dimethylanthracen-2 (9H)-ylidene)-N-methylmethanaminium iodide (23)

1-(3,6-Bis(dimethylamino)-10,10-dimethyl-9,10-dihydroanthracen-9-yl)ethan-1-ol (12) (20 mg, 0.059 mmol, 1 equiv.) was dissolved in CH₂Cl₂— MeCOH 1:1 (2 mL) and p-chloranil (30.1 mg, 0.118 mmol, 2 equiv.) was added to the solution. The mixture was stirred until the LC-MS indicated full conversion. The reaction was evaporated and applied directly to a silica gel column and purified by flash chromatography on silica (Eluent: CH₂Cl₂— MeOH 0% to 20%).

The product was redissolved in CH₂Cl₂, one drop of HI (57% in water) was added, stirred for 5 min and another flash chromatography (on silica, eluent: CH₂Cl₂— MeOH 0% to 20%) was carried out, obtaining the final compound with I⁻ counterion.

Yield: 12 mg, 0.026 mmol, 44%.

¹H NMR (500 MHz, Acetonitrile-d₃) δ 8.49 (d, J=9.6 Hz, 2H), 7.09 (d, J=2.8 Hz, 2H), 6.90 (dd, J=9.7, 2.7 Hz, 2H), 5.89 (q, J=6.9 Hz, 1H), 3.30 (s, 12H), 1.72 (d, J=6.8 Hz, 3H), 1.67 (s, 6H).

¹³C NMR (126 MHz, Acetonitrile-d₃) δ 158.5, 157.0, 152.9, 152.0, 136.5, 119.5, 113.4, 111.7, 67.5, 41.2, 34.3, 25.7.

MS: [M]⁺: calcd for [C₂₂H₂₉N₂O]: 337, found: 337.

### 1.3 Synthesis of Compounds X Wherein W═Br and A=C⁺

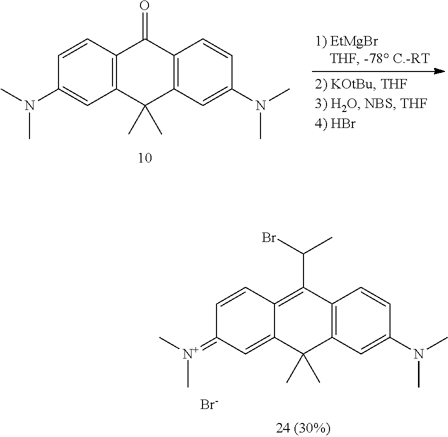

### 1.3.1 Synthesis of N-(10-(1-bromoethyl)-7-(dimethylamino)-9,9-dimethylanthracen-2 (9H)-ylidene)-N-methylmethanaminium bromide (24)

In a dried round-bottom flask of 3,6-bis(dimethylamino)-10,10-dimethylanthracen-9 (10H)-one (10) (93 mg, 0.30 mmol, 1 equiv.) in anhydrous THF (3 mL) was cooled to −78° C. under N₂ atmosphere and EtMgBr (1 M in THF, 2.4 mL, 2.4 mmol, 8 equiv.) was added dropwise. After stirring for 30 min, the cooling bath was removed, and the reaction was stirred for one more hour at room temperature. Then, saturated NH₄Cl (5 mL) was added dropwise, and the mixture was extracted with EtOAc 3 times (3×50 mL). The collected organic phases were washed with brine and dried over Mg₂SO₄. After the evaporation, the dark blue solid was dissolved in anhydrous THF (3 mL) and KO^{t}Bu (1 M in THF, 0.06 mL, 0.06 mmol, 0.2 equiv.) was added to the solution. The colour changed to light blue, then distilled water (0.5 mL) was added, and the reaction was cooled to 0° C. in ice-water bath. Then, N-bromosuccinimide (59 mg, 0.33 mmol, 1.1 equiv.) was added in the dark, for which the reaction turned dark purple. After 15 min stirring at room temperature, 5 drops of cc HBr was added, and the mixture was extracted with CH₂Cl₂ and water. The organic phase was dried over Mg₂SO₄, and the solvent was evaporated. The crude product was purified by flash chromatography (eluent: CH₂Cl₂— MeOH 0% to 20%) to obtain 24 as dark blue crystals.

Yield: 43 mg, 0.090 mmol, 30%.

¹H NMR (500 MHz, Chloroform-d) δ 8.28 (d, J=9.4 Hz, 2H), 7.03 (d, J=2.6 Hz, 2H), 6.98-6.88 (m, 2H), 6.03 (q, J=7.1 Hz, 1H), 3.39 (s, 12H), 2.17 (d, J=7.1 Hz, 3H), 1.67 (s, 6H).

MS: [M]⁺: calcd for [C₂₂H₂₈BrN₂]⁺: 399, found: 399.

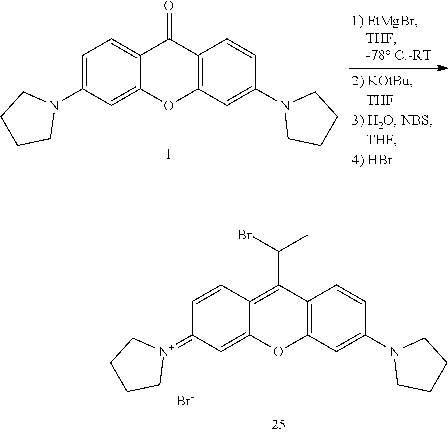 [SMILES: O=c1c2ccc(N3CCCC3)cc2oc2cc(N3CCCC3)ccc12 ; CC(Br)c1c2ccc(=[N+]3CCCC3)cc-2oc2cc(N3CCCC3)ccc12 ; [Br-]]

### 1.3.2. Synthesis of 1-(9-(1-bromoethyl)-6-(pyrrolidin-1-yl)-3H-xanthen-3-ylidene)pyrrolidin-1-ium bromide (25)

In a dried round-bottom flask 3,6-di(pyrrolidin-1-yl)-9H-xanthen-9-one (1) (100 mg, 0.30 mmol, 1 equiv.) in anhydrous THF (5 mL) was cooled to −78° C. under N₂ atmosphere and EtMgBr (1 M in THF, 3.0 mL, 3.0 mmol, 10 equiv.) was added dropwise. After stirring for 30 min, the cooling bath was removed, and the reaction was stirred for one more hour at room temperature. Then, saturated NH₄Cl (5 mL) was added dropwise and the mixture was extracted with EtOAc 3 times (3×50 mL). The collected organic phases were washed with brine and dried over Mg₂SO₄. After the evaporation, the dark pink solid was dissolved in anhydrous THF (3 mL) and KO^{t}Bu (1 M in THF, 0.06 mL, 0.06 mmol, 0.2 equiv.) was added to the solution. The colour changed to light pink, then distilled water (1 mL) was added, and the reaction was cooled to 0° C. in ice-water bath. Then N-bromosuccinimide (59 mg, 0.33 mmol, 1.1 equiv.) was added in dark, for which the reaction turned dark purple. After 15 min stirring at room temperature, 5 drops of cc HBr was added and extracted with CH₂Cl₂ and water. The organic phase was dried over Mg₂SO₄, and the solvent was evaporated. The crude product was purified by flash chromatography (eluent: CH₂Cl₂— MeOH 0% to 20%) to obtain 25 as dark purple crystals.

Yield: 70 mg, 0.138 mmol, 46%.

¹H NMR (500 MHz, Acetonitrile-d₃) δ 8.02 (d, J=9.5 Hz, 1H), 7.04 (dd, J=9.6, 2.5 Hz, 1H), 6.68 (d, J=2.4 Hz, 1H), 3.97-3.91 (m, 8H), 2.18-2.10 (m, 11H).

MS: [M]⁺: calcd for [C₂₃H₂₆BrN₂O]⁺: 425, found: 425.

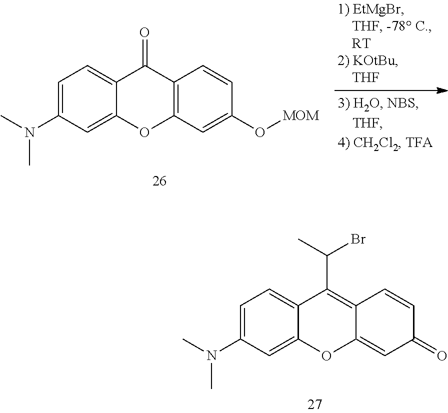

### 1.3.3. Synthesis of 9-(1-bromoethyl)-6-(dimethylamino)-3H-xanthen-3-one (27)

In a dried round-bottom flask 3-(dimethylamino)-6-(methoxymethoxy)-9H-xanthen-9-one (26) (50 mg, 0.167 mmol, 1 equiv.) in anhydrous THF (5 mL) was cooled to −78° C. under N₂ atmosphere and EtMgBr (1 M in THF, 1.67 mL, 1.67 mmol, 10 equiv.) was added dropwise. After stirring for 30 min, the cooling bath was removed, and the reaction was stirred for one more hour at room temperature. Then, saturated NH₄Cl (5 mL) was added dropwise, and the mixture was extracted with EtOAc 3 times (3×50 mL). The collected organic phases were washed with brine and dried over Mg₂SO₄. After the evaporation, the dark pink solid was dissolved in anhydrous THF (3 mL) and KO^{t}Bu (1 M in THF, 35 μL, 0.033 mmol, 0.2 equiv.) was added to the solution. The colour changed to light pink, then distilled water (1 mL) was added, and the reaction was cooled to 0° C. in ice-water bath. Then N-bromosuccinimide (33 mg, 0.184 mmol, 1.1 equiv.) was added in dark, for which the reaction turned dark purple. After 15 min stirring at room temperature, the mixture was extracted with CH₂Cl₂ and water. The organic phase was dried over Mg₂SO₄, and the solvent was evaporated.

The crude product was redissolved in CH₂Cl₂ (1 ml) and TFA (200 μL) was added dropwise. After 30 min stirring, the solvents were evaporated. The crude product was purified by flash chromatography (eluent: CH₂Cl₂— MeOH 0% to 20%) to obtain 27 as dark red crystals.

Yield for 3 steps: 18 mg, 0.0517 mmol, 31%.

¹H NMR (500 MHz, Acetonitrile-d₃, d-TFA) δ 8.43 (d, J=80.4 Hz, 2H), 7.36 (dd, J=10.0, 2.6 Hz, 1H), 7.18 (dd, J=9.3, 2.5 Hz, 1H), 7.11 (d, J=2.5 Hz, 1H), 6.89 (d, J=2.6 Hz, 1H), 6.24 (q, J=7.1 Hz, 1H), 3.44-3.29 (m, 8H), 2.24 (d, J=7.1 Hz, 3H).

MS: [M+H]⁺: calcd for [C₁₇H₁₇BrNO₂]⁺: 346, found: 346.

### 1.4 Synthesis of Compounds X Wherein W=Oxycarbonyl Linker Precursor Moiety Activation of the Hydroxyethyl Derivatives with DSC

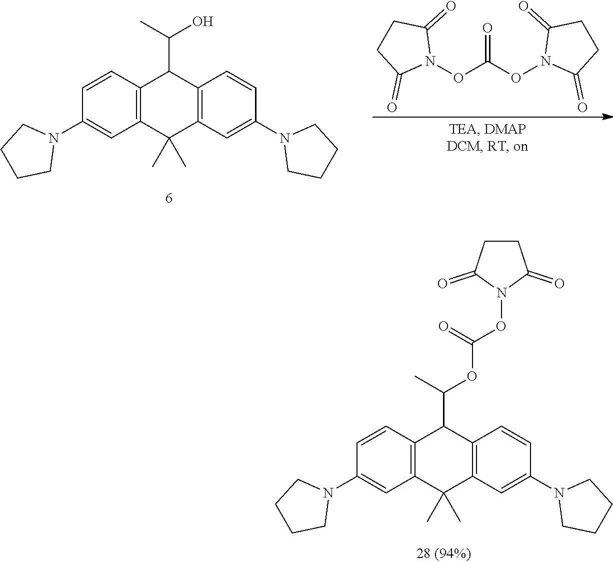 [SMILES: CC(O)C1c2ccc(N3CCCC3)cc2C(C)(C)c2cc(N3CCCC3)ccc21 ; O=C(ON1C(=O)CCC1=O)ON1C(=O)CCC1=O ; CC(OC(=O)ON1C(=O)CCC1=O)C1c2ccc(N3CCCC3)cc2C(C)(C)c2cc(N3CCCC3)ccc21]

### 1.4.1 Synthesis of 1-(10,10-Dimethyl-3,6-di(pyrrolidin-1-yl)-9,10-dihydroanthracen-9-yl)ethyl (2,5-dioxopyrrolidin-1-yl) carbonate (28)

1-(10,10-Dimethyl-3,6-di(pyrrolidin-1-yl)-9,10-dihydroanthracen-9-yl)ethan-1-ol (6) (150 mg, 0.385 mmol, 1 equiv.) was dissolved in acetonitrile (7 mL), DSC (493 mg, 1.92 mmol, 5 equiv.), TEA (536 μL, 3.85 mmol, 10 equiv.) and DMAP (cat.) were added to the solution and stirred for overnight at room temperature. After the LC-MS indicated complete conversion, the reaction was extracted 5 times with saturated NaHCO₃, the organic phase was dried over Mg₂SO₄, and the solvent was evaporated. The crude product was used immediately without further purification.

Yield: 192 mg, 0.362 mmol, 94%.

¹H NMR (500 MHz, Acetonitrile-d₃) δ 7.18 (dd, J=12.7, 8.5 Hz, 2H), 6.77 (dd, J=9.7, 2.5 Hz, 2H), 6.56 (ddd, J=8.5, 3.8, 2.5 Hz, 2H), 5.06-4.96 (m, 1H), 4.26 (d, J=4.7 Hz, 1H), 3.34 (m, J=6.0, 3.9, 3.1 Hz, 8H), 3.16-3.12 (m, 4H), 2.05 (m, J=3.4 Hz, 8H), 1.72 (d, J=8.1 Hz, 6H), 0.98 (d, J=6.4 Hz, 3H).

MS: [M+H]⁺: calcd for [C₃₁H₃₈N₃O₅]⁺: 532, found: 532.

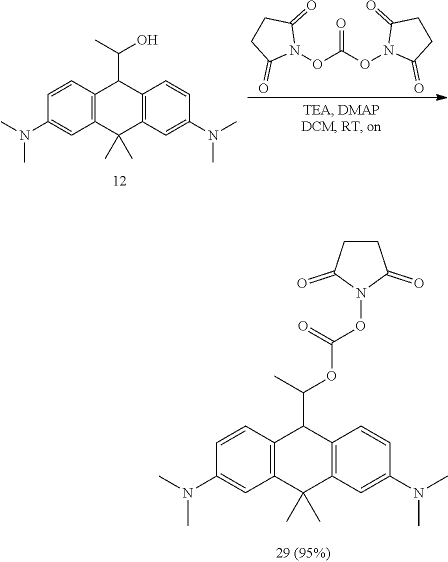

### 1.4.2 Synthesis of 1-(3,6-Bis(dimethylamino)-10,10-dimethyl-9,10-dihydroanthracen-9-yl)ethyl (2,5-dioxopyrrolidin-1-yl) carbonate (29)

1-(3,6-Bis(dimethylamino)-10,10-dimethyl-9,10-dihydroanthracen-9-yl)ethan-1-ol (12) (250 mg, 0.729 mmol, 1 equiv.) was dissolved in acetonitrile (10 mL), DSC (946 mg, 3.69 mmol, 5 equiv.), TEA (1.03 mL, 7.39 mmol, 10 equiv.) and DMAP (cat.) were added to the solution and stirred for overnight at room temperature. After the LC-MS indicated complete conversion, the reaction was extracted 5 times with saturated NaHCO₃, the organic phase was dried over Mg₂SO₄, and the solvent was evaporated. The crude product was used immediately without further purification.

Yield: 352 mg, 0.734 mmol, 99%.

¹H NMR (500 MHz, Acetonitrile-d₃) δ 8.08 (d, J=6.4 Hz, 2H), 7.22-7.10 (m, 2H), 6.90 (t, J=2.6 Hz, 2H), 6.68-6.64 (m, 2H), 2.93 (s, 16H), 1.66 (d, J=4.0 Hz, 9H).

MS: [M+H]⁺: calcd for [C₂₇H₃₄N₃O₅]⁺: 480, found: 480.

### Activation of the hydroxyethyl derivatives with 4-nitrophenyl chloroformate

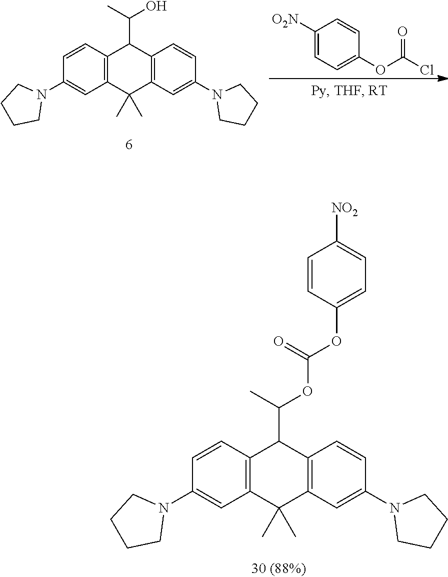 [SMILES: CC(O)C1c2ccc(N3CCCC3)cc2C(C)(C)c2cc(N3CCCC3)ccc21 ; O=C(Cl)Oc1ccc([N+](=O)[O-])cc1 ; CC(OC(=O)Oc1ccc([N+](=O)[O-])cc1)C1c2ccc(N3CCCC3)cc2C(C)(C)c2cc(N3CCCC3)ccc21]

### 1.4.3 Synthesis of 1-(10,10-dimethyl-3,6-di(pyrrolidin-1-yl)-9,10-dihydroanthracen-9-yl)ethyl (4-nitrophenyl) carbonate (30)

1-(10,10-Dimethyl-3,6-di(pyrrolidin-1-yl)-9,10-dihydroanthracen-9-yl)ethan-1-ol (6) (20 mg, 0.051 mmol, 1 equiv.) was dissolved in dry THF and 4-nitrophenyl chloroformate (16 mg, 0.077 mmol, 1.5 equiv.) and pyridine (6.2 μL, 0.077 mmol, 1.5 equiv.) were added to the reaction. After completion, the reaction was extracted with CH₂Cl₂ and water, the organic phase was dried over Mg₂SO₄, and the solvent was evaporated. The crude product was used immediately without further purification.

Yield: 25 mg, 0.045, mmol, 88%.

MS: [M+H]⁺: calcd for [C₃₃H₃₈N₃O₅]⁺: 556, found: 556.

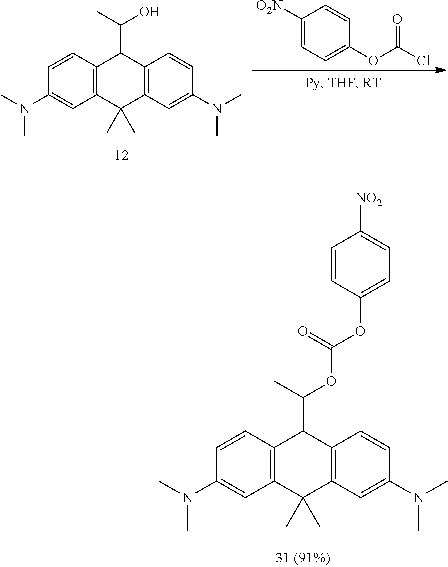

### 1.4.4 Synthesis of 1-(3,6-bis(dimethylamino)-10,10-dimethyl-9,10-dihydroanthracen-9-yl)ethyl (4-nitrophenyl) carbonate (31)

1-(3,6-Bis(dimethylamino)-10,10-dimethyl-9,10-dihydroanthracen-9-yl)ethan-1-ol (12) (25 mg, 0.079 mmol, 1 equiv.) was dissolved in dry THF and 4-nitrophenyl chloroformate (16 mg, 0.079 mmol, 1 equiv.) and pyridine (6.3 μL, 0.079 mmol, 1 equiv.) were added to the reaction. After completion, the reaction was extracted with CH₂Cl₂ and water, the organic phase was dried over Mg₂SO₄, and the solvent was evaporated. The crude product was used immediately without further purification.

Yield: 36 mg, 0.072, mmol, 91%.

¹H NMR (500 MHz, Acetonitrile-d3) δ 8.28 (d, J=9.1 Hz, 2H), 7.35 (d, J=9.2 Hz, 2H), 6.97 (dd, J=6.0, 3.3 Hz, 4H), 6.72 (m, J=7.0, 2.7, 1.4 Hz, 2H), 5.00 (m, J=6.3 Hz, 1H), 4.20 (d, J=5.7 Hz, 1H), 2.97 (s, 12H), 1.70 (s, 6H), 1.11 (d, J=6.4 Hz, 3H).

MS: [M+H]⁺: calcd for [C₂₉H₃₄N₃O₅]⁺: 504, found: 504.

### 1.5 Synthesis of Compounds X Wherein W=Self-Immolative Linker

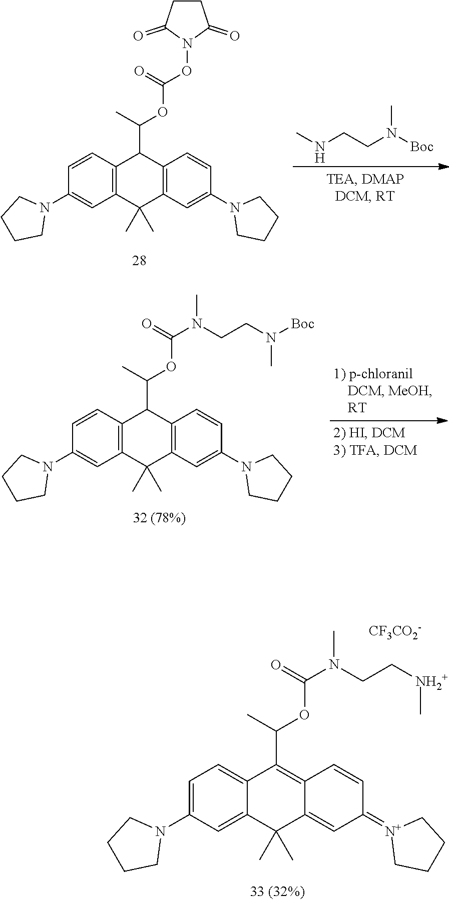 [SMILES: CC(OC(=O)ON1C(=O)CCC1=O)C1c2ccc(N3CCCC3)cc2C(C)(C)c2cc(N3CCCC3)ccc21 ; CNCCN(C)C(=O)OC(C)(C)C ; CC(OC(=O)N(C)CCN(C)C(=O)OC(C)(C)C)C1c2ccc(N3CCCC3)cc2C(C)(C)c2cc(N3CCCC3)ccc21 ; C[NH2+]CCN(C)C(=O)OC(C)C1=C2C=CC(=[N+]3CCCC3)C=C2C(C)(C)c2cc(N3CCCC3)ccc21 ; "CF3CO2-"]

### 1.5.1 Compound 32 (Carbamate Formation)

Compound 28 (100 mg, 0,256 mmol, 1 equiv.) was dissolved in CH₂Cl₂ (3 mL) then tert-butyl methyl(2-(methylamino)ethyl)carbamate (145 mg, 0.768 mmol, 3 equiv.), triethylamine (53 uL, 0.384 mmol, 1.5 equiv.) were added and the reaction was stirred for 1 hour at room temperature. After completion, the reaction was concentrated onto celite and purified by flash chromatography on silica (eluent: hexane-EtOAc 0% to 10%) to afford 32 as a colourless oil. Yield: 120 mg, 0.199 mmol, 78%.

¹H NMR (500 MHz, Chloroform-d) δ 7.32 (t, J=9.4 Hz, 1H), 7.18-7.09 (m, 1H), 6.67 (dd, J=22.7, 2.5 Hz, 2H), 6.56-6.45 (m, 2H), 5.06 (s, 1H), 4.34-4.23 (m, 1H), 3.32 (dd, J=8.4, 5.1 Hz, 8H), 3.06-2.96 (m, 4H), 2.96-2.81 (m, 6H), 2.02 (m, 8H), 1.70 (d, J=11.2 Hz, 6H), 1.46 (s, 9H), 0.72 (d, J=6.4 Hz, 3H).

¹³C NMR (126 MHz, Chloroform-d) δ 146.8, 146.7, 144.7, 129.78, 119.6, 110.6, 109.8, 109.2, 60.4, 47.7, 45.8, 38.5, 34.5, 28.4, 28.4, 25.5, 21.0, 15.5, 14.2.

HRMS: [M+H]⁺: calcd for [C₃₆H₅₃N₄O₄]⁺: 605.4061, found: 605.4052.

### 1.5.2 Compound 33 (Oxidation and Deprotection)

Step 1 (oxidation): Boc-protected compound 32 (120 mg, 0.198 mmol, 1 equiv.) was dissolved in CH₂Cl₂-MeOH-1:1 (3 mL), and chloranil (152 mg, 0.596 mmol, 3 equiv.) was added to the solution. After 30 min, the solvent was evaporated, the dark blue solid was applied directly on silica gel column and purified by flash chromatography (Eluent: CH₂Cl₂— MeOH 0% to 20%).

Step 2 (anion exchange): the purified product was redissolved in CH₂Cl₂ (2 mL), one drop of HI (57% in water) was added, stirred for 5 min and another flash chromatography (on silica) was carried out (eluent: CH₂Cl₂— MeOH 0% to 20%).

Step 3 (deprotection): Boc-protected iodide salt was dissolved in CH₂Cl₂ (1 mL), then trifluoroacetic acid (25% for the mixture) was added and stirred at room temperature for 1 hour. Then volatiles were evaporated and the solid was thoroughly dried. The blue product was used without further purification in the next step.

Yield for the 3 steps: 32%

¹H NMR (500 MHz, Acetonitrile-d₃, d-TFA) δ 8.24 (d, J=9.5 Hz, 2H), 6.98 (d, J=2.4 Hz, 2H), 6.80 (m, 2H), 6.58 (m, 1H), 3.66 (m, 8H), 2.99 (s, 2H), 2.61 (s, 2H), 2.11-2.07 (m, 8H), 1.88 (d, J=7.0 Hz, 3H), 1.65 (s, 6H), 1.27 (s, 6H).

¹³C NMR (126 MHz, Acetonitrile-d₃, d-TFA) δ 163.2, 158.1, 154.2, 135.8, 119.58, 119.57, 115.0, 112.99, 112.97, 72.0, 55.3, 42.8, 34.4, 34.3, 25.9, 22.9.

HRMS: [M]⁺: calcd for [C₃₁H₄₃N₄O₂]⁺: 503.3380, found: 503.3377.

### 2. Synthesis of Target Conjugates (Attachment of Compounds X to the Active Agents)

### 2.1 Synthesis of Model Target Conjugates Wherein the Connecting Chemical Moiety Between the Photoremovable Protecting Group and the Cargo Unit is a Covalent Bond

### Synthesis of Model Target Conjugates from Key Intermediates—Ethers

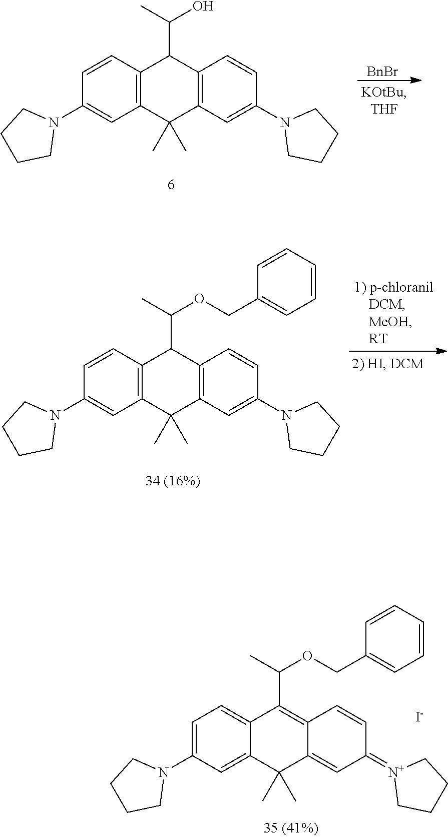 [SMILES: CC(O)C1c2ccc(N3CCCC3)cc2C(C)(C)c2cc(N3CCCC3)ccc21 ; "BnBr" ; CC(OCc1ccccc1)C1c2ccc(N3CCCC3)cc2C(C)(C)c2cc(N3CCCC3)ccc21 ; CC(OCc1ccccc1)C1=C2C=CC(=[N+]3CCCC3)C=C2C(C)(C)c2cc(N3CCCC3)ccc21 ; [I-]]

### 2.1.1 Synthesis of 1,1′-(10-(1-(benzyloxy)ethyl)-9,9-dimethyl-9,10-dihydroanthracene-2,7-diyl)dipyrrolidine (34)

Compound 6 (50 mg, 0.128 mmol, 1 equiv.) was dissolved in dry THF (2 mL), then potassium tert-butoxide (22 mg, 0.192 mmol, 1.5 equiv.) and benzyl bromide (26 mg, 0.154 mmol 1.2 equiv.) were added. The reaction was stirred for 10 min at room temperature, then distilled water was added and the mixture was extracted with CH₂Cl₂ 3 times (3×50 mL) and dried over Mg₂SO₄. The solution was concentrated onto celite and purified by flash chromatography on silica (eluent: hexane-EtOAc 0% to 10%) to afford 34 as a colourless oil. Yield: 10 mg, 0.021 mmol, 16%.

¹H NMR (500 MHz, Chloroform-d) δ 7.35-7.31 (m, 6H), 7.10 (d, J=8.4 Hz, 1H), 6.69 (t, J=2.8 Hz, 2H), 6.51 (ddd, J=8.4, 3.5, 2.5 Hz, 2H), 4.59 (d, J=17.4 Hz, 2H), 4.21 (d, J=4.3 Hz, 1H), 3.69 (td, J=6.3, 4.4 Hz, 1H), 3.33 (dt, J=6.5, 3.3 Hz, 8H), 2.02 (q, J=3.3 Hz, 8H), 1.70 (d, J=20.6 Hz, 6H), 0.77 (d, J=6.2 Hz, 3H).

¹³C NMR (126 MHz, cdcl₃) δ 146.7, 146.6, 145.3, 145.1, 139.3, 132.4, 130.4, 129.8, 128.4, 128.2, 127.8, 127.6, 127.2, 122.6, 121.0, 110.3, 110.1, 109.3, 109.1, 82.5, 70.7, 47.8, 45.9, 38.6, 34.9, 34.0, 25.4, 16.2.

MS: [M+H]⁺: calcd for [C₃₃H₄₁N₂O]⁺: 481, found: 481.

### 2.1.2 Synthesis of 1-(10-(1-(benzyloxy)ethyl)-9,9-dimethyl-7-(pyrrolidin-1-yl)anthracen-2 (9H)-ylidene)pyrrolidin-1-ium iodide (35)

Compound 34 (10 mg, 0.021 mmol, 1 equiv.) was dissolved in 1 mL CH₂Cl₂— MeOH 1:1 in the dark and p-chloranil (21 mg, 0.083 mmol, 4 equiv.) was added to the solution. The mixture was stirred until the LC-MS indicated full conversion (30 min). The reaction mixture was evaporated and applied directly to a silica gel column and purified by flash chromatography on silica (eluent: CH₂Cl₂— MeOH 0% to 20%). The product was redissolved in CH₂Cl₂, one drop of HI (57% in water) was added, the mixture was stirred for 5 min and another flash chromatography (on silica, eluent: CH₂Cl₂— MeOH 0% to 20%) was carried out, obtaining the final compound as blue crystals with I⁻ counterion. Yield: 5 mg, 0.008 mmol, 41%. HRMS: [M]⁺: calcd for [C₃₃H₃₉N₂O]⁺: 479.3057, found: 479.3064.

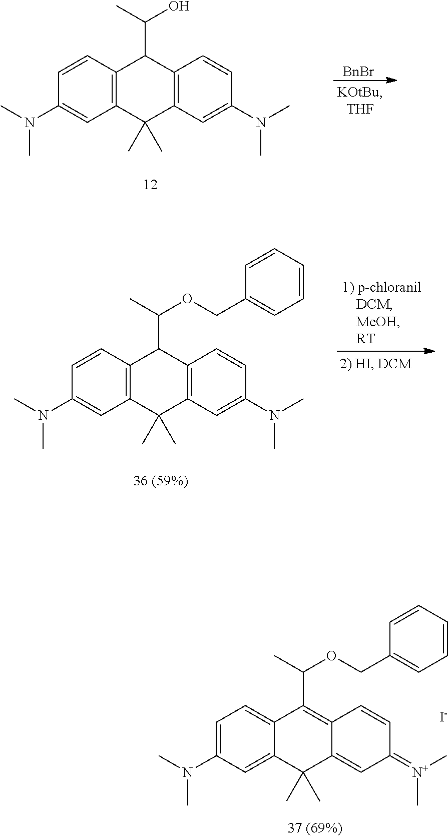

### 2.1.310-(1-(Benzyloxy)ethyl)-N2,N2,N7,N7,9,9-hexamethyl-9,10-dihydroanthracene-2,7-diamine (36)

Compound 12 (30 mg, 0.088 mmol, 1 equiv.) was dissolved in dry THF (2 mL), then potassium tert-butoxide (20 mg, 0.176 mmol, 2 equiv.) and benzyl bromide (16 μL, 0.132 mmol 1.5 equiv.) were added. The reaction was stirred for 10 min at room temperature, then distilled water was added and the mixture was extracted with CH₂Cl₂ 3 times (3×50 mL) and dried over Mg₂SO₄. The solution was concentrated onto celite and purified by flash chromatography on silica (eluent: hexane-EtOAc 0% to 10%) to afford 36 as a colourless oil.

Yield: 22 mg, 0.052 mmol, 59%

¹H NMR (500 MHz, Chloroform-d) δ 7.29-7.24 (m, 5H), 7.23-7.19 (m, 1H), 7.06 (d, J=8.4 Hz, 1H), 6.83 (dd, J=4.5, 2.7 Hz, 2H), 6.63 (ddd, J=8.4, 4.4, 2.7 Hz, 2H), 4.57-4.47 (m, 2H), 4.15 (d, J=4.4 Hz, 1H), 3.63 (qd, J=6.3, 4.5 Hz, 1H), 2.91 (s, 12H), 1.66 (s, 3H), 1.61 (s, 3H), 0.73 (d, J=6.3 Hz, 3H).

¹³C NMR (126 MHz, Chloroform-d) δ 149.5, 149.4, 145.2, 145.0, 139.1, 130.2, 129.7, 128.3, 127.7, 127.2, 124.0, 122.5, 111.5, 111.4, 110.9, 110.7, 82.2, 70.7, 46.0, 41.1, 41.0, 38.7, 34.9, 33.9, 16.2.

MS: [M+H]⁺: calcd for [C₂₉H₃₇N₂O]⁺: 429, found: 429.

### 2.1.4 N-(10-(1-(Benzyloxy)ethyl)-7-(dimethylamino)-9,9-dimethylanthracen-2 (9H)-ylidene)-N-methylmethanaminium iodide (37)

Compound 36 (30 mg, 0.088 mmol, 1 equiv.) was dissolved in 1 mL CH₂Cl₂— MeOH 1:1 in the dark and p-chloranil (43 mg, 0.176 mmol, 2 equiv.) was added to the solution. The mixture was stirred until the LC-MS indicated full conversion (30 min). The reaction mixture was evaporated and applied directly to a silica gel column and purified by flash chromatography on silica (eluent: CH₂Cl₂— MeOH 0% to 20%). The product was redissolved in CH₂Cl₂, one drop of HI (57% in water) was added, the mixture was stirred for 5 min and another flash chromatography (on silica, eluent: CH₂Cl₂— MeOH 0% to 20%) was carried out, obtaining the final compound as blue crystals with I-counterion.

Yield: 20 mg, 0.036 mmol, 69%

¹H NMR (500 MHz, Acetonitrile-d₃) δ 8.47 (d, J=9.6 Hz, 2H), 7.34-7.21 (m, 5H), 7.11 (d, J=2.7 Hz, 2H), 6.90 (dd, J=9.7, 2.7 Hz, 2H), 5.66 (q, J=6.8 Hz, 1H), 4.54-4.39 (m, 2H), 3.32 (s, 12H), 1.77 (d, J=6.9 Hz, 3H), 1.69 (s, 6H).

¹³C NMR (126 MHz, Acetonitrile-d₃) δ 165.7, 157.3, 156.1, 137.9, 134.9, 128.4, 128.0, 127.8, 118.9, 112.7, 110.9, 74.5, 71.6, 41.9, 40.3, 33.4, 23.0.

HRMS: [M]⁺: calcd for [C₂₉H₃₅N₂O]⁺: 427.2744, found: 427.2747.

### Synthesis of Photocages from Key Intermediates—Esters

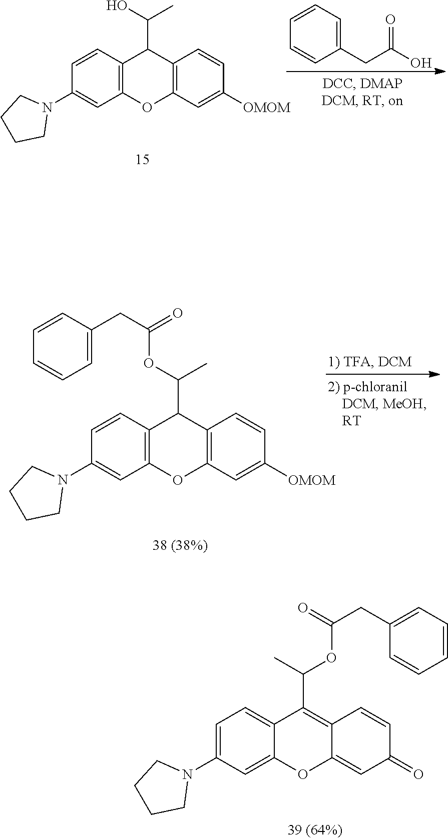

2.1.5 Synthesis of 1-(3-(methoxymethoxy)-6-(pyrrolidin-1-yl)-9H-xanthen-9-yl)ethyl 2-phenylacetate (38) Hydroxyethyl derivative 15 (26 mg, 0.073 mmol, 1 equiv.) was dissolved in 1 ml CH₂Cl₂ then phenylacetic acid (15 mg, 0.11 mmol, 1.5 equiv.), DCC (18 mg, 0.087 mmol, 1.2 equiv.) and DMAP (catalytic amount) were added to the solution and the resulting mixture was stirred for 30 min. After completion, the reaction mixture was concentrated onto celite and purified by flash chromatography on silica (eluent: hexane-EtOAc, 0% to 10%) affording 38 as a colourless oil. Yield: 12 mg, 0.027 mmol, 38%.

### Mixture of Two Diastereomers (Double Integrals):

¹H NMR (500 MHz, Chloroform-d) δ 7.37-7.33 (m, 4H), 7.31-7.28 (m, 6H), 7.16 (d, J=8.5 Hz, 1H), 7.10 (d, J=8.4 Hz, 1H), 6.78-6.75 (m, 2H), 6.75-6.70 (m, 3H), 6.63-6.59 (m, 1H), 6.31 (dd, J=8.4, 2.4 Hz, 1H), 6.27-6.20 (m, 3H), 5.18-5.15 (m, 4H), 5.07-4.99 (m, 2H), 4.05 (d, J=3.9 Hz, 2H), 3.66-3.62 (m, 4H), 3.50 (s, 3H), 3.49 (s, 3H), 3.31-3.26 (m, 8H), 2.03-1.98 (m, 8H), 0.90-0.86 (m, 6H).

¹³C NMR (126 MHz, Chloroform-d) δ 171.0, 157.0, 153.7, 153.5, 153.5, 153.4, 153.0, 152.2, 148.2, 137.1, 134.1, 134.1, 130.6, 130.3, 129.9, 129.7, 129.4, 128.6, 128.6, 127.1, 127.0, 115.7, 114.1, 111.2, 111.0, 108.0, 107.5, 107.4, 107.3, 106.7, 104.1, 103.8, 98.8, 98.6, 94.6, 76.3, 56.0, 47.7, 47.7, 42.0, 41.9, 41.9, 41.8, 36.6, 25.5, 25.5, 24.7, 23.3, 14.7, 14.4.

HRMS: [M+H]⁺: calcd for [C₂₉H₃₂NO₅]⁺: 474.2274, found: 474.2267.

### 2.1.6 Synthesis of 1-(3-oxo-6-(pyrrolidin-1-yl)-3H-xanthen-9-yl)ethyl 2-phenylacetate (39)

Deprotection (step 1): compound 38 (35 mg, 0.074 mmol, 1 equiv.) was dissolved in CH₂Cl₂ (2 mL), and TFA (500 uL) was added and stirred at room temperature for 1 hour. After the completion of the deprotection, the volatiles were evaporated and the solid was thoroughly dried.

Oxidation (step 2): the resulting solid was dissolved in 1 mL CH₂Cl₂— MeOH 1:1 and p-chloranil (36 mg, 0.148 mmol, 2 equiv.) was added to the solution in the dark. The mixture was stirred until the LC-MS indicated full conversion (30 min). Then, the reaction mixture was evaporated and applied directly to a silica gel column and purified by flash chromatography on silica (eluent: CH₂Cl₂—MeOH 0% to 20%) affording 39 as a red crystalline solid. Yield for the two steps: 20 mg, 0.048 mmol, 64%.

¹H NMR (500 MHz, Acetonitrile-d₃; d-TFA) δ 8.23 (d, J=9.9 Hz, 1H), 8.18 (d, J=9.9 Hz, 1H), 7.30-7.25 (m, 3H), 7.20-7.16 (m, 2H), 7.07-6.98 (m, 3H), 6.73-6.67 (m, 2H), 3.78-3.72 (m, 2H), 3.71 (s, 2H), 3.67-3.60 (m, 2H), 2.15-2.10 (m, 4H), 1.80 (d, J=7.0 Hz, 3H).

¹³C NMR (126 MHz, Acetonitrile-d₃; d-TFA) δ 171.6, 166.8, 159.6, 159.2, 157.9, 157.2, 134.6, 131.1, 130.9, 130.4, 129.6, 128.2, 119.4, 117.6, 115.8, 113.5, 103.7, 97.9, 69.5, 51.0, 50.8, 41.5, 25.9, 25.6, 21.4.

HRMS: [M+H]⁺: calcd for [C₂₇H₂₆NO₄]⁺: 427.1784, found: 427.2747.

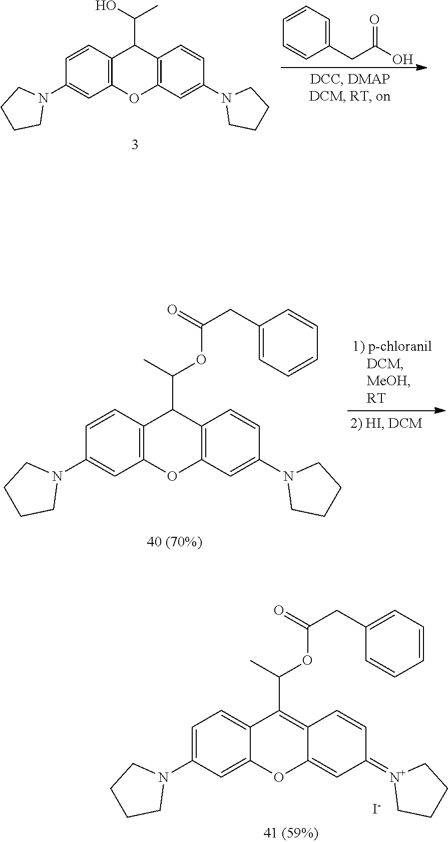 [SMILES: CC(O)C1c2ccc(N3CCCC3)cc2Oc2cc(N3CCCC3)ccc21 ; O=C(O)Cc1ccccc1 ; CC(OC(=O)Cc1ccccc1)C1c2ccc(N3CCCC3)cc2Oc2cc(N3CCCC3)ccc21 ; CC(OC(=O)Cc1ccccc1)c1c2ccc(=[N+]3CCCC3)cc-2oc2cc(N3CCCC3)ccc12 ; [I-]]

### 2.1.7 Synthesis of 1-(3,6-di(pyrrolidin-1-yl)-9H-xanthen-9-yl)ethyl2-phenylacetate (40)

Hydroxyethyl derivative 3 (50 mg, 0.137 mmol, 1 equiv.) was dissolved in 2 mL CH₂Cl₂ then phenylacetic acid (28 mg, 0.205 mmol, 1.5 equiv.), DCC (34 mg, 0.165 mmol, 1.2 equiv.) and DMAP (catalytic amount) were added to the solution and the resulting mixture was stirred for 30 min. After completion, the reaction mixture was concentrated onto celite and purified by flash chromatography on silica (eluent: hexane-EtOAc, 0% to 10%) affording 40 as a colourless oil.

Yield: 46 mg, 0.095 mmol, 70%.

### Mixture of Two Diastereomers:

¹H NMR (500 MHz, Chloroform-d) δ 7.38-7.28 (m, 5H), 7.12 (d, J=8.3 Hz, 1H), 6.76 (d, J=8.3 Hz, 1H), 6.30 (dd, J=8.4, 2.4 Hz, 1H), 6.27 (d, J=2.3 Hz, 2H), 6.23 (dd, J=8.4, 2.5 Hz, 1H), 5.08-5.02 (m, 1H), 4.04 (d, J=3.9 Hz, 1H), 3.66 (d, J=3.0 Hz, 2H), 3.32-3.27 (m, 8H), 2.05-1.97 (m, 8H), 0.89 (d, J=6.4 Hz, 3H).

¹³C NMR (126 MHz, Chloroform-d) δ 171.1, 153.9, 153.5, 148.2, 134.4, 130.5, 129.8, 129.6, 129.3, 128.7, 128.7, 127.1, 109.0, 107.5, 107.3, 107.1, 98.9, 98.8, 76.8, 47.8, 42.1, 41.7, 25.6, 14.6.

HRMS: [M+H]⁺: calcd for [C₃₁H₃₅N₂O₃]⁺: 483.2642, found: 483.2624.

### 2.1.8 Synthesis of 1-(9-(1-(2-phenylacetoxy)ethyl)-6-(pyrrolidin-1-yl)-3H-xanthen-3-ylidene)pyrrolidin-1-ium iodide (41)

Compound 40 (83 mg, 0.172 mmol, 1 equiv.) was dissolved in 2 mL CH₂Cl₂— MeOH 1:1 and p-chloranil (170 mg, 0.688 mmol, 4 equiv.) was added to the solution. The mixture was stirred until the LC-MS indicated full conversion (30 min). Then, the reaction mixture was evaporated and applied directly to a silica gel column and purified by flash chromatography on silica (eluent: CH₂Cl₂— MeOH 0% to 20%). The product was redissolved in CH₂Cl₂, one drop of HI (57% in water) was added, stirred for 5 min and another flash chromatography (on silica) was carried out (eluent: CH₂Cl₂— MeOH 0% to 20%) affording 41 as purple crystals. Yield: 62 mg, 0.102 mmol, 59%.

¹H NMR (500 MHz, Acetonitrile-d₃) δ 8.08 (d, J=9.6 Hz, 2H), 7.31-7.23 (m, 3H), 7.21-7.17 (m, 2H), 6.85 (dd, J=9.5, 2.4 Hz, 2H), 6.64 (q, J=7.0 Hz, 1H), 6.59 (d, J=2.4 Hz, 2H), 3.70 (s, 2H), 3.65-3.50 (m, 8H), 2.11-2.07 (m, 8H), 1.78 (d, J=7.0 Hz, 3H).

¹³C NMR (126 MHz, Acetonitrile-d₃) δ 170.5, 157.6, 156.4, 154.4, 133.7, 129.4, 129.2, 128.6, 127.1, 115.2, 111.0, 96.6, 68.4, 48.9, 40.6, 24.9, 20.5.

HRMS: [M]⁺: calcd for [C₃₁H₃₃N₂O₃]⁺: 481.2486, found: 481.2493.

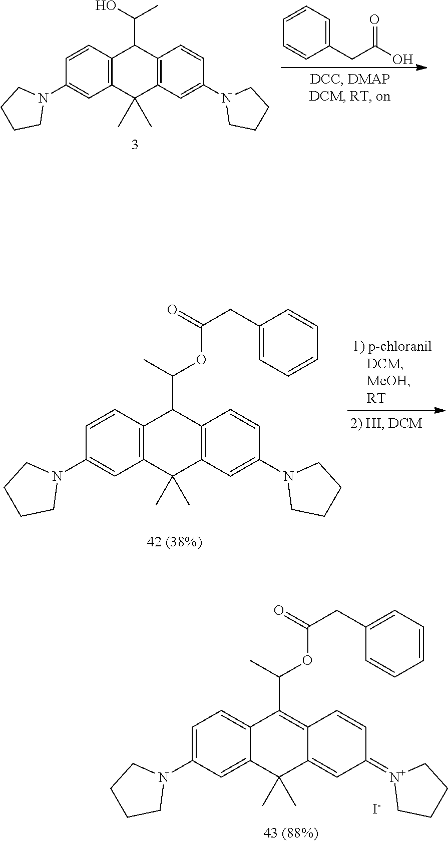 [SMILES: CC(O)C1c2ccc(N3CCCC3)cc2C(C)(C)c2cc(N3CCCC3)ccc21 ; O=C(O)Cc1ccccc1 ; CC(OC(=O)Cc1ccccc1)C1c2ccc(N3CCCC3)cc2C(C)(C)c2cc(N3CCCC3)ccc21 ; CC(OC(=O)Cc1ccccc1)C1=C2C=CC(=[N+]3CCCC3)C=C2C(C)(C)c2cc(N3CCCC3)ccc21 ; [I-]]

### 2.1.9 Synthesis of 1-(10,10-dimethyl-3,6-di(pyrrolidin-1-yl)-9,10-dihydroanthracen-9-yl)ethyl 2-phenylacetate (42)

Hydroxyethyl derivative 3 (162 mg, 0.416 mmol, 1 equiv.) was dissolved in 5 mL CH₂Cl₂ then phenylacetic acid (85 mg, 0.624 mmol, 1.5 equiv.), DCC (103 mg, 0.499 mmol, 1.2 equiv.) and DMAP (catalytic amount) were added to the solution and the resulting mixture was stirred for 30 min. After completion, the reaction mixture was concentrated onto celite and purified by flash chromatography on silica (eluent: hexane-EtOAc, 0% to 10%) affording 42 as a colourless oil.

Yield: 60 mg, 0.118 mmol, 28%.

¹H NMR (500 MHz, Chloroform-d) δ 7.38-7.27 (m, 5H), 7.23 (d, J=8.4 Hz, 1H), 6.94 (d, J=8.4 Hz, 1H), 6.68 (d, J=2.5 Hz, 1H), 6.65 (d, J=2.5 Hz, 1H), 6.48 (dd, J=8.4, 2.4 Hz, 1H), 6.41 (dd, J=8.4, 2.5 Hz, 1H), 5.17-5.08 (m, 1H), 4.12 (d, J=4.7 Hz, 1H), 3.67 (d, J=4.3 Hz, 2H), 3.38-3.32 (m, 8H), 2.06-1.98 (m, 8H), 1.69 (d, J=3.4 Hz, 7H), 0.80 (d, J=6.4 Hz, 3H).

¹³C NMR (126 MHz, Chloroform-d) δ 171.2, 147.0, 147.0, 145.9, 145.1, 134.5, 130.3, 130.0, 129.6, 129.4, 128.7, 128.7, 127.1, 121.5, 119.9, 110.6, 110.2, 110.1, 109.4, 109.4, 77.9, 47.9, 46.4, 42.2, 38.8, 35.1, 34.0, 25.6, 15.9.

HRMS: [M+H]⁺: calcd for [C₃₄H₄₁N₂O₂]⁺: 509.3162, found: 509.3154.

### 2.1.10 Synthesis of 1-(9,9-dimethyl-10-(1-(2-phenylacetoxy)ethyl)-7-(pyrrolidin-1-yl)anthracen-2 (9H)-ylidene)pyrrolidin-1-ium iodide (43)

Compound 42 (26 mg, 0.043 mmol, 1 equiv.) was dissolved in 1 ml CH₂Cl₂— MeOH 1:1 and p-chloranil (21 mg, 0.086 mmol, 2 equiv.) was added to the solution. The mixture was stirred until the LC-MS indicated full conversion (30 min). Then, the reaction mixture was evaporated and applied directly to a silica gel column and purified by flash chromatography on silica (eluent: CH₂Cl₂— MeOH 0% to 20%). The product was redissolved in CH₂Cl₂, one drop of HI (57% in water) was added, stirred for 5 min and another flash chromatography (on silica, eluent: CH₂Cl₂— MeOH 0% to 20%) was carried out, obtaining the final compound as blue crystals with I⁻ counterion. Yield: 24 mg, 0.038 mmol, 88%.

¹H NMR (500 MHz, Acetonitrile-d₃) δ 8.07 (d, J=9.5 Hz, 2H), 7.26-7.20 (m, 3H), 7.13 (dd, J=7.4, 2.1 Hz, 2H), 6.93 (d, J=2.5 Hz, 2H), 6.63 (dd, J=9.5, 2.5 Hz, 2H), 6.58 (q, J=7.1 Hz, 1H), 3.67-3.61 (m, 8H), 2.11-2.07 (m, 8H), 1.82 (d, J=7.1 Hz, 3H), 1.61 (s, 6H).

¹³C NMR (126 MHz, Acetonitrile-d₃) δ 170.4, 162.1, 156.9, 153.2, 134.44, 134.43, 133.7, 129.2, 128.5, 127.0, 118.0, 113.5, 111.5, 69.9, 48.9, 41.7, 40.8, 24.8, 21.2.

HRMS: [M]⁺: calcd for [C₃₄H₃₉N₂O₂]⁺: 507.3006, found: 507.3006.

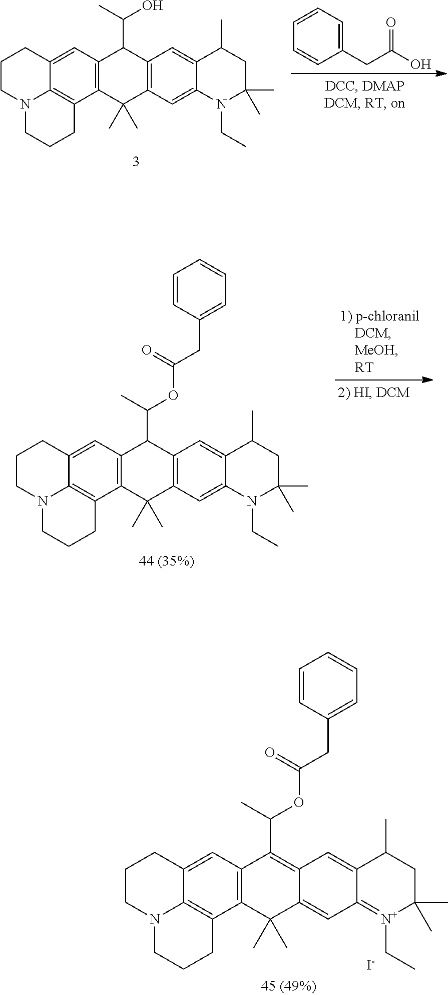 [SMILES: CCN1c2cc3c(cc2C(C)CC1(C)C)C(C(C)O)c1cc2c4c(c1C3(C)C)CCCN4CCC2 ; O=C(O)Cc1ccccc1 ; CCN1c2cc3c(cc2C(C)CC1(C)C)C(C(C)OC(=O)Cc1ccccc1)c1cc2c4c(c1C3(C)C)CCCN4CCC2 ; CC[N+]1=c2cc3c(cc2C(C)CC1(C)C)=C(C(C)OC(=O)Cc1ccccc1)c1cc2c4c(c1C3(C)C)CCCN4CCC2 ; [I-]]

### 2.1.11 Synthesis of Compound 44

Hydroxyethyl derivative 9 (104 mg, 0.220 mmol, 1 equiv.) was dissolved in 5 mL CH₂Cl₂ then phenylacetic acid (45 mg, 0.330 mmol, 1.5 equiv.), DCC (55 mg, 0.264 mmol, 1.2 equiv.) and DMAP (catalytic amount) were added to the solution and the resulting mixture was stirred for 30 min. After completion, the reaction mixture was concentrated onto celite and purified by flash chromatography on silica (eluent: hexane-EtOAc, 0% to 10%) affording 44 as a colourless oil.

Yield: 45 mg, 0.076 mmol, 35%.

### Mixture of Four Diastereomers:

¹H NMR (500 MHz, Chloroform-d) δ 7.38-7.25 (m, 5H), 7.12-6.53 (m, 3H), 5.17-4.99 (m, 1H), 4.19-4.12 (m, 2H), 3.74-3.44 (m, 5H), 3.30-3.12 (m, 4H), 3.05-2.83 (m, 2H), 2.79-2.62 (m, 1H), 2.01-1.89 (m, 4H), 1.88-1.84 (m, 3H), 1.82-1.78 (m, 2H), 1.78-1.73 (m, 3H), 1.42-1.16 (m, 12H), 0.78-0.71 (m, 3H).

MS: [M+H]⁺: calcd for [C₄₀H₅₁N₂O₂]⁺: 591, found: 591.

### 2.1.12 Synthesis of Target Conjugate 45

Compound 44 (22 mg, 0.037 mmol, 1 equiv.) was dissolved in 1 ml CH₂Cl₂— MeOH 1:1 and p-chloranil (28 mg, 0.112 mmol, 3 equiv.) was added to the solution. The mixture was stirred until the LC-MS indicated full conversion (30 min). Then, the reaction mixture was evaporated and applied directly to a silica gel column and purified by flash chromatography on silica (eluent: CH₂Cl₂— MeOH 0% to 20%). The product was redissolved in CH₂Cl₂, one drop of HI (57% in water) was added, stirred for 5 min and another flash chromatography (on silica, eluent: CH₂Cl₂— MeOH 0% to 20%) was carried out, obtaining the final compound as blue crystals with I-counterion.

Yield: 13 mg, 0.018 mmol, 49%.

¹H NMR (500 MHz, Acetonitrile-d₃-TFA-d) δ 7.96-7.86 (m, 1H), 7.80-7.75 (m, 1H), 7.26-7.18 (m, 3H), 7.13-7.05 (m, 2H), 6.90-6.82 (m, 1H), 6.71-6.61 (m, 1H), 3.77-3.50 (m, 8H), 3.11-3.06 (m, 2H), 2.94-2.68 (m, 3H), 2.02-1.96 (m, 2H), 1.87-1.81 (m, 3H), 1.77-1.71 (m, 6H), 1.48-1.44 (m, 3H), 1.38-1.26 (m, 12H).

MS: [M]⁺: calcd for [C₄₀H₄₉N₂O₂]⁺: 589, found: 589.

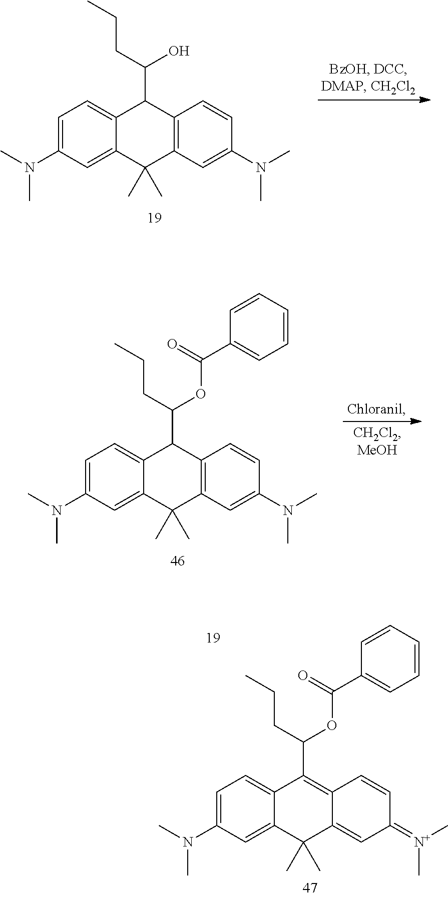

### 2.1.13 Synthesis of 1-(3,6-bis(dimethylamino)-9H-xanthen-9-yl)butyl benzoate (46)

Hydroxyethyl derivative (19) (16 mg, 0.044 mmol, 1 equiv.) was dissolved in 2 mL CH₂CO₂ then benzoic acid (11 mg, 0.088 mmol, 2 equiv.), DCC (15 mg, 0.075 mmol, 1.7 equiv.) and DMAP (catalytic amount) were added to the solution and the resulting mixture was stirred for 30 min. After completion, the reaction mixture was concentrated onto celite and purified by flash chromatography on silica (eluent: hexane-EtOAc, 0% to 10%) affording compound 46 as a colourless oil.

Yield: 10 mg, 0.0225 mmol, 51%.

### Mixture of Two Diastereomers:

¹H NMR (500 MHz, Chloroform-d) δ 8.19-8.13 (m, 2H), 7.63-7.56 (m, 1H), 7.50 (t, J=7.7 Hz, 2H), 7.43 (d, J=8.5 Hz, 1H), 7.17 (d, J=8.6 Hz, 1H), 6.90 (d, J=2.7 Hz, 1H), 6.86 (d, J=2.6 Hz, 1H), 6.68 (td, J=9.0, 2.6 Hz, 2H), 5.35 (dt, J=8.5, 4.3 Hz, 1H), 4.38 (d, J=4.2 Hz, 1H), 2.99 (d, J=13.3 Hz, 12H), 1.73 (d, J=25.7 Hz, 6H), 1.31-1.19 (m, 2H), 1.18-1.13 (m, 2H), 0.68 (t, J=7.2 Hz, 3H).

MS: [M+H]⁺: calcd for [C₂₈H₃₃N₂O₃]⁺: 445, found: 445.

### 2.1.14 Synthesis of N-(9-(1-(benzoyloxy)butyl)-6-(dimethylamino)-3H-xanthen-3-ylidene)-N-methylmethanaminium (47)

Compound 46 (10 mg, 0.021 mmol, 1 equiv.) was dissolved in 1 ml CH₂Cl₂— MeOH 1:1 and p-chloranil (12 mg, 0.050 mmol, 2.5 equiv.) was added to the solution. The mixture was stirred until the LC-MS indicated full conversion (30 min). Then, the reaction mixture was evaporated and applied directly to a silica gel column and purified by flash chromatography on silica (eluent: CH₂Cl₂— MeOH 0% to 20%), obtaining the final compound 47 as blue crystals.

Yield: 6 mg, 0.0135 mmol, 64%.

¹H NMR (500 MHz, Chloroform-d) δ 8.06-8.01 (m, 2H), 7.65-7.58 (m, 1H), 7.48 (t, J=7.8 Hz, 2H), 7.06 (d, J=2.6 Hz, 2H), 6.88 (dd, J=9.7, 2.6 Hz, 2H), 6.77 (dd, J=9.4, 5.2 Hz, 1H), 3.37 (s, 12H), 2.48 (m, 1H), 2.10 (m, 1H), 1.76-1.70 (m, 1H), 1.68 (s, 6H), 1.52 (m, 1H), 1.04 (t, J=7.4 Hz, 3H).

MS: [M]⁺: calcd for [C₂₈H₃₁N₂O₃]⁺: 444, found: 444.

### 2.2 Synthesis of Model Target Conjugates Wherein the Connecting Chemical Moiety is an Oxycarbonyl Linker

### Synthesis of Model Target Conjugates from Key Intermediates—Carbamates

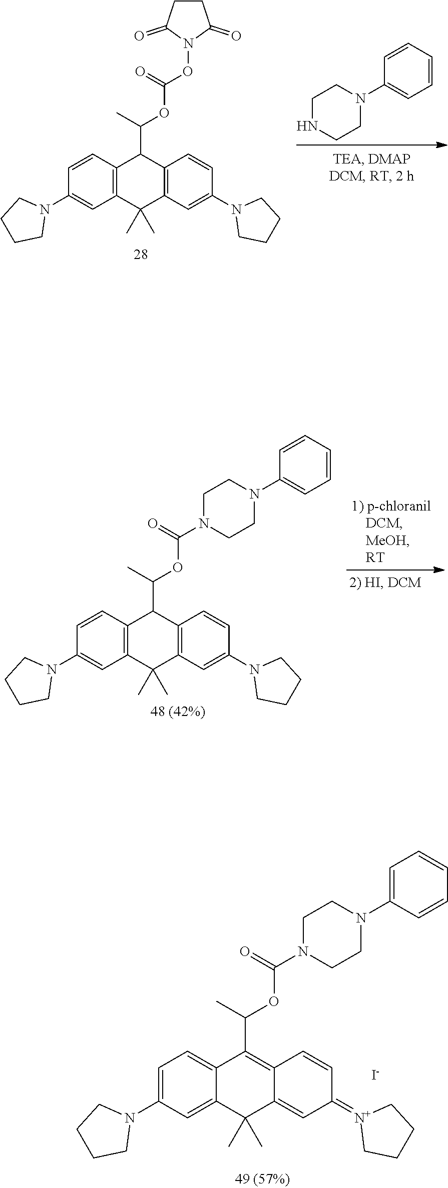 [SMILES: CC(OC(=O)ON1C(=O)CCC1=O)C1c2ccc(N3CCCC3)cc2C(C)(C)c2cc(N3CCCC3)ccc21 ; c1ccc(N2CCNCC2)cc1 ; CC(OC(=O)N1CCN(c2ccccc2)CC1)C1c2ccc(N3CCCC3)cc2C(C)(C)c2cc(N3CCCC3)ccc21 ; CC(OC(=O)N1CCN(c2ccccc2)CC1)C1=C2C=CC(=[N+]3CCCC3)C=C2C(C)(C)c2cc(N3CCCC3)ccc21 ; [I-]]

### 2.2.1 Synthesis of 1-(10,10-dimethyl-3,6-di(pyrrolidin-1-yl)-9,10-dihydroanthracen-9-yl)ethyl 4-phenylpiperazine-1-carboxylate (48)

Compound 28 (177 mg, 0,128 mmol, 1 equiv.) was dissolved in 5 mL CH₂Cl₂ (stabilized with amylene) then N-phenylpiperazine (30 μL, 0.192 mmol, 1.5 equiv.), TEA (53 μL, 0.384 mmol, 3 equiv.) and DMAP (15 mg, 0.128 mmol, 1 equiv.) were added and the was stirred for 1 hour at room temperature. After completion, the mixture was concentrated onto celite in vacuo and purified by flash chromatography on silica (eluent: hexane-EtOAc 0% to 10%) to afford 48 as a colourless oil.

Yield: 31 mg, 0.054 mmol, 42%.

### Mixture of Two Diastereomers:

¹H NMR (500 MHz, Acetonitrile-d₃) δ 7.27 (dd, J=8.8, 7.2 Hz, 2H), 7.19 (d, J=8.4 Hz, 1H), 7.11 (d, J=8.3 Hz, 1H), 7.00-6.95 (m, 2H), 6.89-6.83 (m, 1H), 6.73 (d, J=2.5 Hz, 1H), 6.70 (d, J=2.5 Hz, 1H), 6.52-6.46 (m, 2H), 4.93-4.83 (m, 1H), 4.09 (d, J=5.6 Hz, 1H), 3.70-3.45 (m, 4H), 3.31-3.26 (m, 8H), 3.18-3.07 (m, 4H), 2.03-1.97 (m, 8H), 1.66 (d, J=1.6 Hz, 6H), 0.79 (d, J=6.5 Hz, 3H).

¹³C NMR (126 MHz, Chloroform-d) δ 155.2, 151.5, 147.0, 147.0, 146.9, 146.0, 145.0, 130.4, 129.9, 129.4, 121.8, 120.5, 116.9, 116.9, 110.7, 110.2, 109.9, 109.5, 109.4, 78.4, 61.6, 49.7, 49.6, 47.9, 46.2, 38.8, 34.8, 34.5, 25.6, 25.6, 15.9, 14.8.

HRMS: [M+H]⁺: calcd for [C₃₇H₄₇N₄O₂]⁺: 579.3693, found: 579.3686.

### 2.2.2 Synthesis of 1-(9,9-dimethyl-10-(1-((4-phenylpiperazine-1-carbonyl)oxy)ethyl)-7-(pyrrolidin-1-yl)anthracen-2 (9H)-ylidene)pyrrolidin-1-ium (49)

Compound 48 (10 mg, 0.017 mmol, 1 equiv.) was dissolved in 1 mL CH₂Cl₂— MeOH 1:1 in the dark and p-chloranil (17 mg, 0.068 mmol, 4 equiv.) was added to the solution. The mixture was stirred until the LC-MS indicated full conversion (30 min). The reaction mixture was evaporated and applied directly to a silica gel column and purified by flash chromatography on silica (Eluent: CH₂Cl₂— MeOH 0% to 20%). The product was redissolved in CH₂Cl₂ (1 mL), one drop of HI (57% in water) was added, the mixture was stirred for 5 min and another flash chromatography (on silica, eluent: CH₂Cl₂— MeOH 0% to 20%) was carried out, obtaining the final compound as blue crystals with I⁻ counterion. Yield: 7 mg, 0.010 mmol, 57%.

¹H NMR (500 MHz, Acetonitrile-d₃) δ 8.28 (d, J=9.5 Hz, 2H), 7.27-7.23 (m, 2H), 6.97 (d, J=2.5 Hz, 2H), 6.94 (d, J=8.2 Hz, 2H), 6.86 (t, J=7.3 Hz, 1H), 6.80 (dd, J=9.6, 2.5 Hz, 2H), 6.56 (q, J=7.1 Hz, 1H), 3.80-2.85 (m, 8H and 4H), 2.10-2.07 (m, 8H), 1.87 (d, J=7.1 Hz, 3H), 1.65 (s, 6H).

¹³C NMR (126 MHz, Acetonitrile-d₃) δ 158.1, 154.8, 154.3, 135.7, 135.6, 130.6, 119.2, 114.7, 112.6, 71.5, 49.9, 42.8, 34.1, 30.4, 25.8, 22.5.

HRMS: [M]⁺: calcd for [C₃₇H₄₅N₄O₂]⁺: 577.3537, found: 577.3538.

### Synthesis of Model Target Conjugates from Key Intermediates—Carbonates

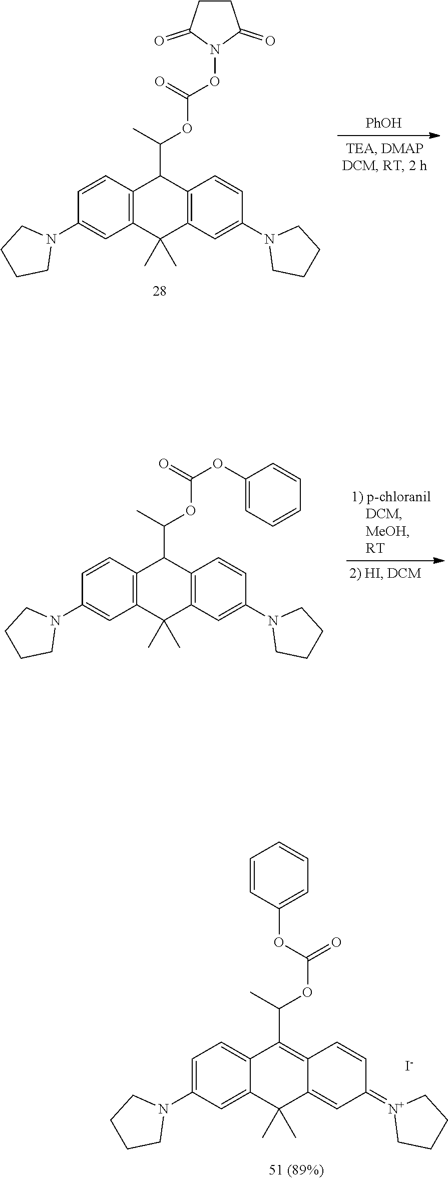 [SMILES: CC(OC(=O)ON1C(=O)CCC1=O)C1c2ccc(N3CCCC3)cc2C(C)(C)c2cc(N3CCCC3)ccc21 ; "PhOH" ; CC(OC(=O)Oc1ccccc1)C1c2ccc(N3CCCC3)cc2C(C)(C)c2cc(N3CCCC3)ccc21 ; CC(OC(=O)Oc1ccccc1)C1=C2C=CC(=[N+]3CCCC3)C=C2C(C)(C)c2cc(N3CCCC3)ccc21 ; [I-]]

### Synthesis of 1-(10,10-dimethyl-3,6-di(pyrrolidin-1-yl)-9,10-dihydroanthracen-9-yl)ethyl phenyl carbonate (50)

Compound 51 (50 mg, 0.128 mmol, 1 equiv.) was dissolved in CH₂Cl₂ (3 mL) then phenol (18 mg, 0.192 mmol, 1.5 equiv.), triethylamine (53 μL, 0.384 mmol, 3 equiv.) and DMAP (16 mg, 0.128 mmol, 1 equiv.) was added and the reaction was stirred for 1 hour at room temperature. After completion, the reaction was concentrated onto celite and purified by flash chromatography on silica (eluent: hexane-EtOAc 0% to 10%) to afford 50 as a colourless oil.

Yield: 32 mg, 0.063 mmol, 49%.

### Mixture of Two Diastereomers:

¹H NMR (500 MHz, Acetonitrile-d₃) δ 7.39 (dd, J=8.5, 7.4 Hz, 2H), 7.28-7.24 (m, 1H), 7.18 (d, J=8.3 Hz, 1H), 7.14 (d, J=8.4 Hz, 1H), 7.11-7.07 (m, 2H), 6.73 (dd, J=9.0, 2.5 Hz, 2H), 6.55-6.47 (m, 2H), 4.90 (q, J=6.3 Hz, 1H), 4.11 (d, J=5.7 Hz, 1H), 3.32-3.27 (m, 8H), 2.03-1.98 (m, 8H), 1.68 (s, 6H), 1.03 (d, J=6.4 Hz, 3H).

¹³C NMR (126 MHz, Acetonitrile-d₃) δ 154.0, 152.3, 148.5, 148.4, 147.0, 146.4, 131.1, 130.8, 130.6, 130.4, 127.0, 122.4, 122.3, 121.6, 120.7, 111.2, 111.1, 110.4, 110.3, 82.5, 48.5, 48.5, 48.0, 39.7, 35.2, 33.5, 26.2, 25.7, 17.4.

HRMS: [M+H]⁺: calcd for [C₃₃H₃₉N₂O₃]⁺: 511.2955, found: 511.2951.

### Synthesis of 1-(9,9-dimethyl-10-(1-((phenoxycarbonyl)oxy)ethyl)-7-(pyrrolidin-1-yl)anthracen-2 (9H)-ylidene)pyrrolidin-1-ium iodide (51)

Compound 50 (32 mg, 0.063 mmol, 1 equiv.) was dissolved in 1 mL CH₂Cl₂—MeOH 1:1 in the dark and p-chloranil (64 mg, 0.251 mmol, 4 equiv.) was added to the solution. The mixture was stirred until the LC-MS indicated full conversion (30 min). The reaction mixture was evaporated and applied directly to a silica gel column and purified by flash chromatography on silica (eluent: CH₂Cl₂—MeOH 0% to 20%).

The product was redissolved in CH₂Cl₂, one drop of HI (57% in water) was added, the mixture was stirred for 5 min and another flash chromatography (on silica, eluent: CH₂Cl₂—MeOH 0% to 20%) was carried out, obtaining the final compound as blue crystals with I⁻ counterion. Yield: 34 mg, 0.0534 mmol, 89%.

¹H NMR (500 MHz, Acetonitrile-d₃) δ 8.21 (d, J=9.5 Hz, 2H), 7.38 (t, J=7.9 Hz, 2H), 7.27 (t, J=7.4 Hz, 1H), 7.12-7.05 (m, 2H), 7.00 (d, J=2.5 Hz, 2H), 6.82 (dd, J=9.5, 2.5 Hz, 2H), 6.64 (q, J=7.0 Hz, 1H), 3.72-3.62 (m, 8H), 2.11-2.06 (m, 8H), 1.95 (d, J=1.8 Hz, 3H), 1.66 (s, 6H).

¹³C NMR (126 MHz, Acetonitrile-d₃) δ 160.3, 157.1, 153.4, 152.6, 151.0, 134.5, 129.7, 126.4, 121.2, 120.9, 118.1, 113.9, 111.9, 73.7, 49.0, 41.9, 24.8, 21.4.

HRMS: [M]⁺: calcd for [C₃₃H₃₇N₂O₃]⁺: 509.2799, found: 509.2802.

### 2.3 Synthesis of Target Conjugates Wherein the Connecting Chemical Moiety Between the Photoremovable Protecting Group and the Cargo Unit is a Self-Immolative Linker

### Synthesis of Photocage-SN38 Conjugates

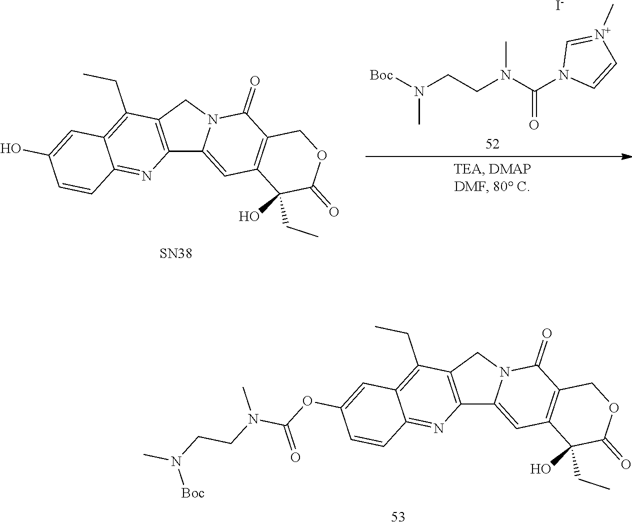 [SMILES: CCc1c2c(nc3ccc(O)cc13)-c1cc3c(c(=O)n1C2)COC(=O)[C@]3(O)CC ; CN(CCN(C)C(=O)n1cc[n+](C)c1)C(=O)OC(C)(C)C ; [I-] ; CCc1c2c(nc3ccc(OC(=O)N(C)CCN(C)C(=O)OC(C)(C)C)cc13)-c1cc3c(c(=O)n1C2)COC(=O)[C@]3(O)CC ; "SN38"]

### 2.3.1 Synthesis of Compound 53

SN-38 (177 mg, 0.45 mmol, 1 equiv.) was dissolved in dry DMF, then compound 52 (230 mg, 0.54 mmol, 1.2 equiv.)³⁹, triethylamine (136 μL, 1.35 mmol, 3 equiv.) and DMAP (2 mg, 0.035 mmol, 0.03 equiv.) was added under N₂ atmosphere. The reaction was stirred at 80° C. for 2 hours. The solvent was evaporated, concentrated onto silica gel and purified by flash chromatography on silica (eluent: CH₂Cl₂—MeOH 0% to 20%).

Yield: 400 mg, 0.356 mmol, 79%.

¹H NMR (500 MHz, DMSO-d₆) δ 8.21-8.17 (m, 1H), 7.96 (s, 1H), 7.66-7.59 (m, 1H), 7.34 (s, 1H), 6.50 (s, 1H), 5.44 (s, 2H), 5.35 (s, 2H), 3.63 (t, J=5.1 Hz, 1H), 3.52-3.42 (m, 3H), 3.23-3.13 (m, 4H), 2.90 (s, 3H), 2.74 (s, 3H), 1.93-1.82 (m, 2H), 1.45-1.36 (m, 9H), 1.34-1.29 (m, 3H), 0.89 (t, J=7.3 Hz, 3H).

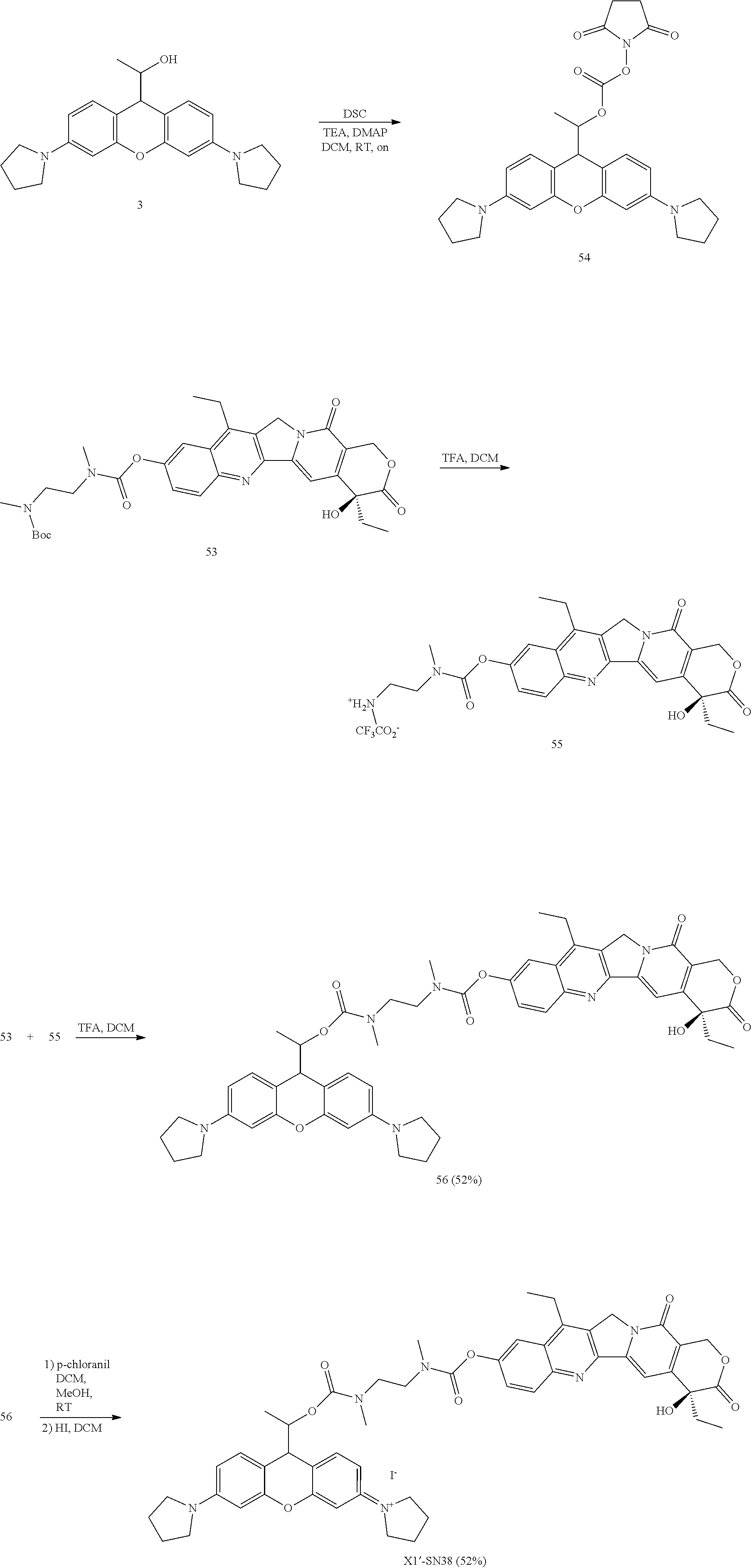

### 2.3.2 Synthesis of Compound X1′-SN38

Compound 54: 1-(3,6-di(pyrrolidin-1-yl)-9H-xanthen-9-yl)ethan-1-ol (3) (100 mg, 0.274 mmol, 1 equiv.) was dissolved in acetonitrile (5 mL), DSC (352 mg, 1.37 mmol, 5 equiv.), TEA (382 μL, 2.74 mmol, 10 equiv.) and DMAP (cat.) were added to the solution and stirred for overnight at room temperature. After the LC-MS indicated complete conversion, the reaction was extracted 5 times with saturated NaHCO₃, the organic phase was dried over Mg₂SO₄, and the solvent was evaporated. The crude product was used immediately without further purification.

Yield: 136 mg, 0.270 mmol, 99%.

MS: [M+H]⁺: calcd for [C₂₈H₃₂N₃O₆]⁺: 506, found: 506.

Compound 56: SN38-Boc-protected self-immolative linker (53) (166 mg, 0.274 mmol, 1 equiv.) was dissolved in CH₂Cl₂ and TFA (25% for the mixture) was added and stirred at room temperature for 1 hour. After the completion of the deprotection, the volatiles were evaporated and the solid was thoroughly dried.

The succinimidyl carbonate (54) (0.274 mmol, 1 equiv.) was dissolved in CH₂Cl₂, the SN-38-self-immolative linker and triethylamine (114 μL, 0.822 mmol, 3 equiv.) was added, and stirred for 1 hour at room temperature. The reaction was concentrated onto silica gel and purified by flash chromatography on silica (eluent: CH₂Cl₂—MeOH 0% to 20%). The compound was dissolved in acetonitrile and purified once more by reverse-phase chromatography (eluent: water-Acetonitrile 5% to 100%).

Yield: 127 mg, 0.142 mmol, 52%.

MS: [M+H]⁺: calcd for [C₅₁H₅₇N₆O₉]⁺: 897, found: 897.

Compound X1′-SN38: the reduced photocage (56) (30 mg, 0.034 mmol, 1 equiv.) was dissolved in CH₂Cl₂—MeOH 1-1 mL) and p-chloranil (17 mg, 0.067 mmol, 2 equiv.) was added to the solution. The mixture was stirred until the LC-MS indicated full conversion (30 min). The reaction was evaporated and applied directly to a silica gel column and purified by flash chromatography on silica (Eluent: CH₂Cl₂—MeOH 0% to 20%).

The product was redissolved in CH₂Cl₂, one drop of HI (57% in water) was added, stirred for 5 min and another flash chromatography (on silica) was carried out, obtaining the final compound with I-counterion (eluent: CH₂Cl₂—MeOH 0% to 20%). Yield: 18 mg, 0.018 mmol, 52%.

¹H NMR (500 MHz, Acetonitrile-d₃) δ 8.43-8.02 (m, 5H), 7.57-7.49 (m, 1H), 7.01-6.90 (m, 2H), 6.72-6.51 (m, 1H), 6.13 (s, 1H), 5.72-5.64 (m, 1H), 5.51-5.30 (m, 3H), 3.65-3.46 (m, 11H), 3.33-3.14 (m, 4H), 3.08 (d, J=2.7 Hz, 2H), 3.02 (s, 2H), 2.96-2.89 (m, 2H), 2.17-2.07 (m, 11H), 1.90-1.81 (m, 4H), 1.42-1.29 (m, 4H), 1.24-1.17 (m, 2H), 1.09-1.00 (m, 4H).

HRMS: [M]⁺: calcd for [C₅₁H₅₅N₆O₉]⁺: 895.4025, found: 895.4028.

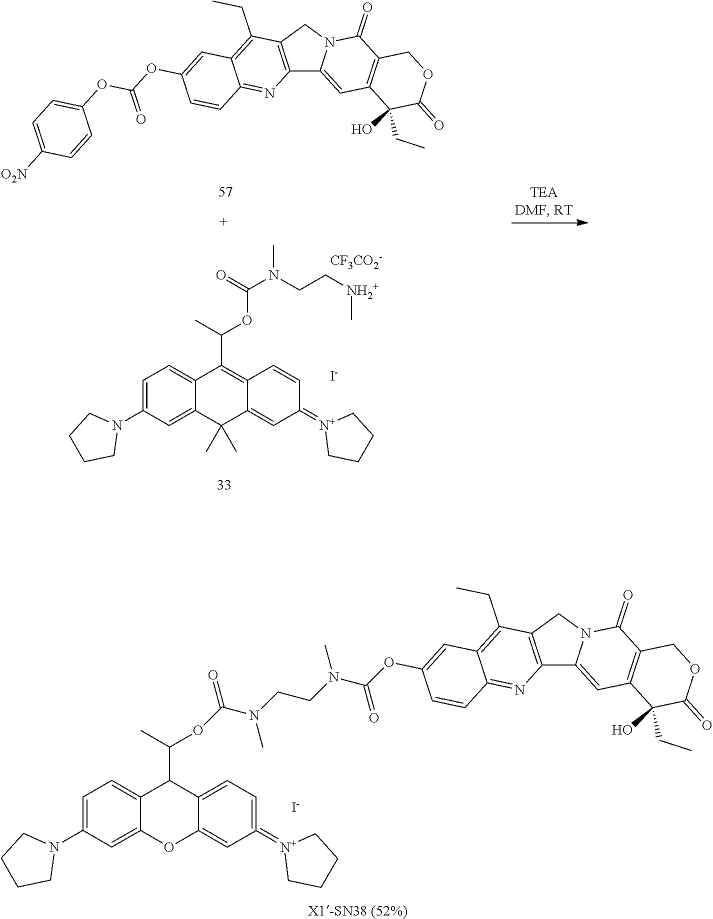

### 2.3.3 Synthesis of Compound X2′-SN38

Compound 57 (112 mg, 0.198 mmol, 1 equiv.) was dissolved in dry DMF, then crude compound 33 (120 mg, 0.198 mmol, 1 equiv.), and DIPEA (69 μL, 0.396 mmol, 2 equiv.) were added and the reaction mixture was stirred for 30 min at room temperature. After the LC-MS indicated full conversion, the solvent was evaporated, the reaction was concentrated onto silica gel and purified by flash chromatography on silica gel (eluent: CH₂Cl₂—MeOH 0% to 20%) to obtain compound X2′-SN38 as a blue solid. Yield: 107 mg, 0,116 mmol, 58%

¹H NMR (500 MHz, Acetonitrile-d₃) δ 8.35-7.93 (m, 3H), 7.88-7.47 (m, 2H), 6.95-6.76 (m, 2H), 6.78-6.50 (m, 3H), 5.61 (d, J=16.6 Hz, 1H), 5.38 (d, J=16.6 Hz, 1H), 5.37-5.29 (m, 2H), 3.64-3.48 (m, 11H), 3.30-3.06 (m, 5H), 3.03 (s, 1H), 2.97-2.76 (m, 3H), 2.10-2.01 (m, 9H), 1.88-1.77 (m, 3H), 1.66-1.51 (m, 4H), 1.46 (d, J=5.3 Hz, 2H), 1.38-1.21 (m, 5H), 1.02-0.93 (m, 3H).

HRMS: [M]⁺: calcd for [C₅₄H₆₁N₆O₈]⁺: 921.4545, found: 921.4551.

### 2.4 Comparative Examples

In these examples R7═H, in which case the expected compounds could not be isolated in their dye form, only in the form of a chloranil complex. In this form, only the exo-form is present, which is demonstrated by the LC-MS chromatograms, where the mass is equal to the mass of the photocage and the chloranil together (with the characteristic isotope distribution of four Cl atoms of the chloranil). NMR characterisation also showed that the double bond is located outside of the ring system (singlet peak in the region of 7-9 ppm), also confirming that R7=H compounds are only stable in the form of a complex that in fact does not absorb in the visible range, thus cannot be used as visible light photocages. This observation is in accordance with the results of Kln et al.³¹

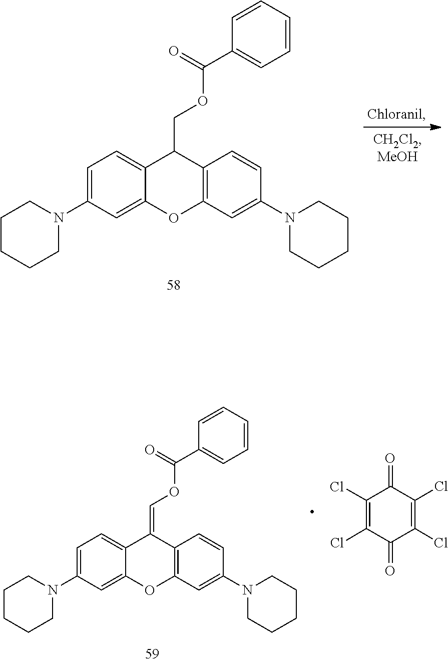 [SMILES: O=C(OCC1c2ccc(N3CCCCC3)cc2Oc2cc(N3CCCCC3)ccc21)c1ccccc1 ; O=C(OC=C1c2ccc(N3CCCCC3)cc2Oc2cc(N3CCCCC3)ccc21)c1ccccc1 ; O=C1C(Cl)=C(Cl)C(=O)C(Cl)=C1Cl]

### 2.4.1 Synthesis of (3,6-di(piperidin-1-yl)-9H-xanthen-9-ylidene)methyl benzoate chloranil complex (59)

Compound 58 (18 mg, 0.037 mmol, 1 equiv.) was dissolved in 1 mL CH₂Cl₂—MeOH 1:1 in the dark and p-chloranil (14 mg, 0.055 mmol, 1.5 equiv.) was added to the solution. The mixture was stirred for 1 h at room temperature. The LC-MS showed full conversion to the chloranil complex of the photocage. The solvent was evaporated and purified by reverse-phase column chromatography (Eluent: Water-MeCN-0.1% TFA).

The expected product (dye-form of the photocage) could not be isolated, only in the form of the chloranil complex in exo-form.

¹H NMR (500 MHz, Chloroform-d) δ 8.49 (d, J=9.7 Hz, 2H), 8.10 (s, 1H), 8.02-7.92 (m, 2H), 7.64-7.57 (m, 1H), 7.44 (t, J=7.8 Hz, 2H), 7.21 (dd, J=9.8, 2.5 Hz, 2H), 6.98 (d, J=2.6 Hz, 2H), 3.77 (d, J=5.2 Hz, 8H), 1.80 (d, J=3.2 Hz, 8H).

MS for the chloranil complex: [M+H]⁺: calcd for [C₃₇H₃₃Cl₄N₂O₅]⁺: 725, found: 725.

MS for the exo-form: [M+H]⁺: calcd for [C₃₁H₃₃N₂O₃]⁺: 481, found: 481.

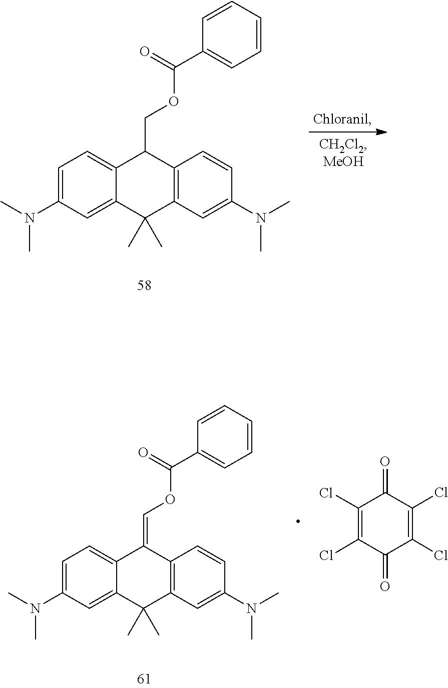

### 2.4.2 Synthesis of (3,6-bis(dimethylamino)-10,10-dimethylanthracen-9 (10H)-ylidene)methyl benzoate chloranil complex (61)

Compound 60 (20 mg, 0.0462 mmol, 1 equiv.) was dissolved in 1 mL CH₂Cl₂—MeOH 1:1 in the dark and p-chloranil (28 mg, 0.115 mmol, 2.5 equiv.) was added to the solution. The mixture was stirred for 1 h at room temperature. The LC-MS showed full conversion to the chloranil complex of the photocage. The solvent was evaporated and purified by reverse-phase column chromatography (Eluent: Water-MeCN-0.1% TFA).

The expected product (dye-form of the photocage) could not be isolated, only in the form of the chloranil complex in exo-form.

¹H NMR (500 MHz, Chloroform-d) δ 8.59 (d, J=9.6 Hz, 2H), 8.19 (s, 1H), 8.05-7.98 (m, 2H), 7.64-7.59 (m, 1H), 7.46 (t, J=7.7 Hz, 2H), 7.14 (d, J=2.6 Hz, 2H), 6.95 (dd, J=9.7, 2.6 Hz, 2H), 3.41 (s, 12H), 1.74 (s, 6H).

MS for the chloranil complex: [M+H]⁺: calcd for [C₃₄H₃₁Cl₄N₂O₄]⁺: 671, found: 671.

MS for the exo-form: [M+H]⁺: calcd for [C₂₈H₃₁N₂O₂]⁺: 427, found: 427.

### 3. Spectroscopic Properties of the Target Conjugates

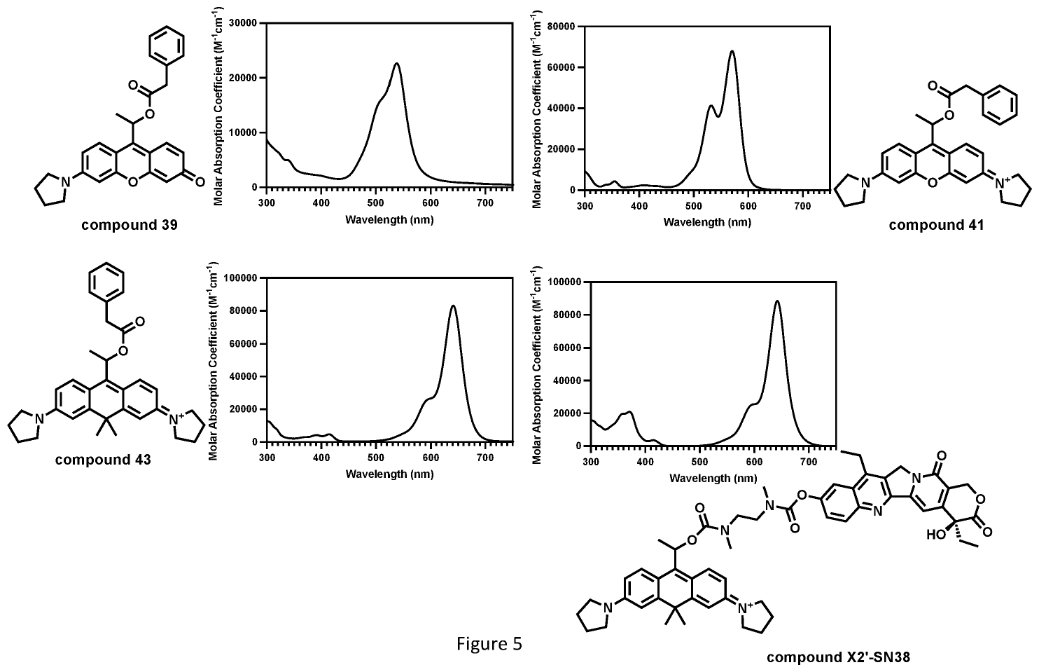

The spectroscopic properties of the target conjugates were determined in PBS. All derivatives have one-photon absorption in the visible range and no signs of aggregation was observed (absorbance measured up to 25 μM) that demonstrates good water solubility for all compounds. Compounds with Q=O have absorption bands in the green-yellow region while Q=CMe₂ derivatives absorb in the red (up to 700 nm). All of the compounds have moderate to high absorption coefficients and significant fluorescence. The spectroscopic details can be found in Table 1. In the table, λₘₐₓ means absorption maximum; λₑₘ means fluorescence emission maximum; s means molar absorption coefficient (at the absorption maximum). Furthermore, FIG. 5 shows the structure and absorption spectra of selected target conjugates.

TABLE 1
Optical properties of selected compounds

| Compound | λₘₐₓ (nm) | λₑₘ (nm) | ε (M⁻¹cm⁻¹) |
|---|---|---|---|
| 35 | 635 | 659 | 81 200 |
| 39 | 538 | 564 | 22 700 |
| 41 | 570 | 592 | 70 000 |
| 43 | 641 | 674 | 86 000 |
| 45 | 660 | 680 | 83 800 |
| 49 | 637 | 673 | 75 100 |
| 51 | 643 | 676 | 73 300 |
| X1′-SN38 | 575 | 594 | 61 200 |
| X2′-SN38 | 642 | 660 | 88 400 |

### 4. Uncaging Studies of the Target Conjugates

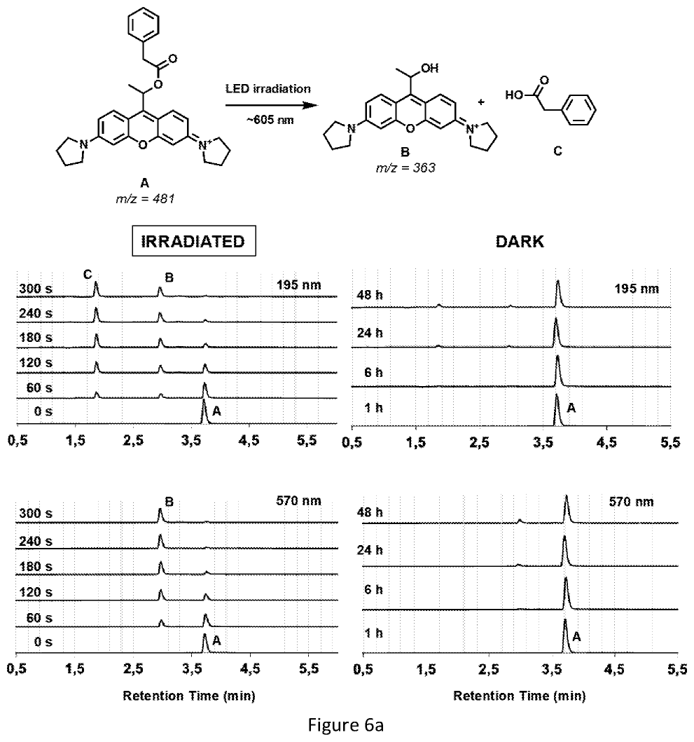

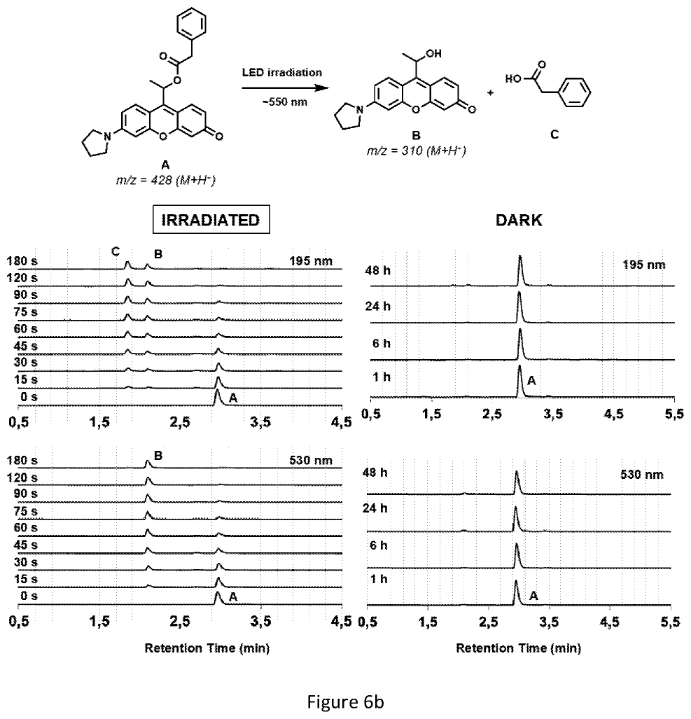

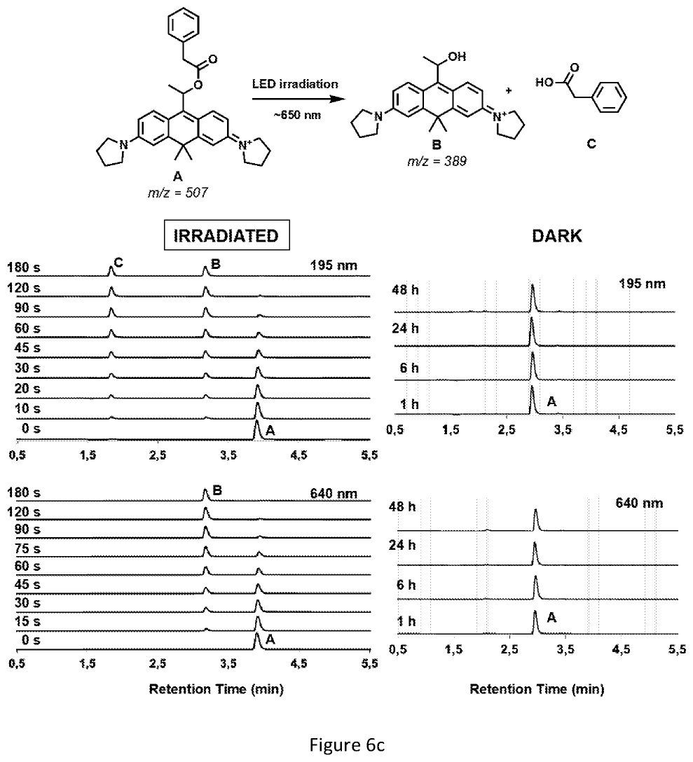

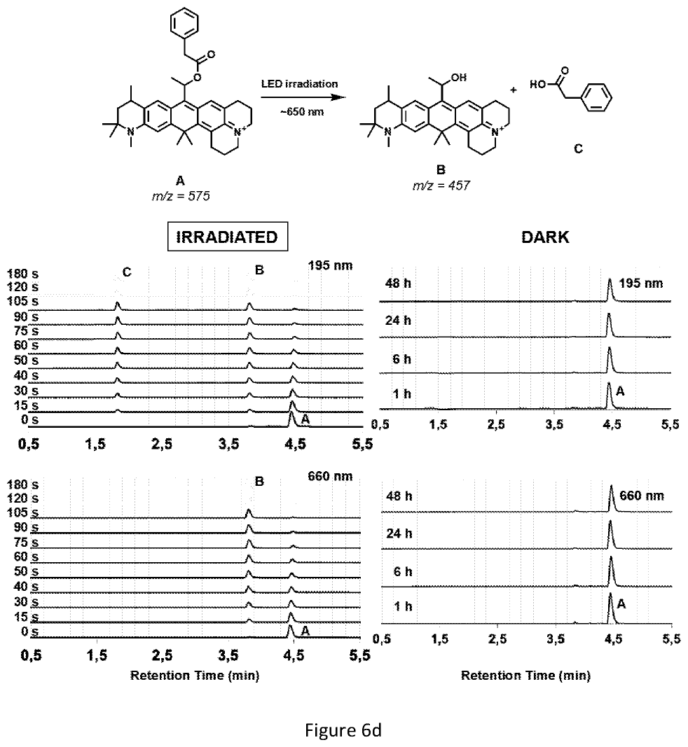

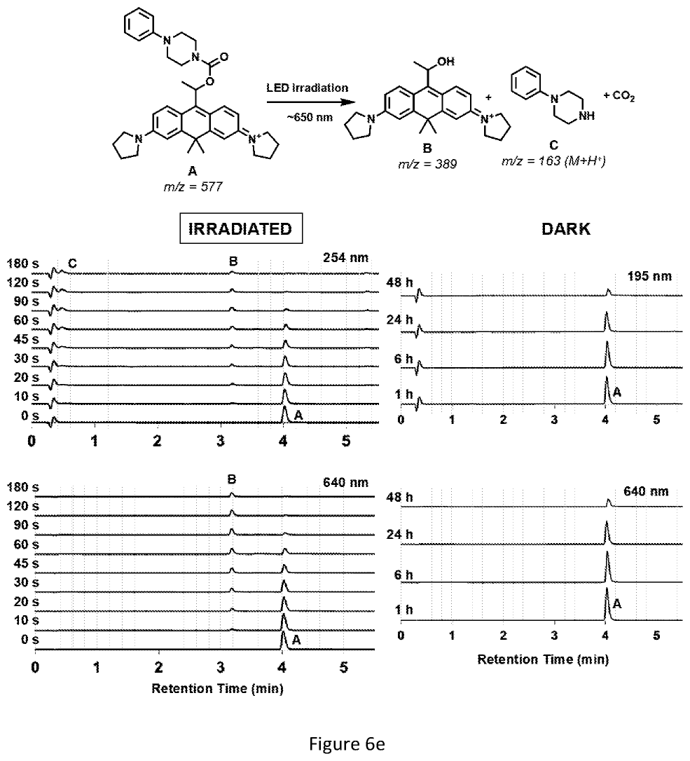

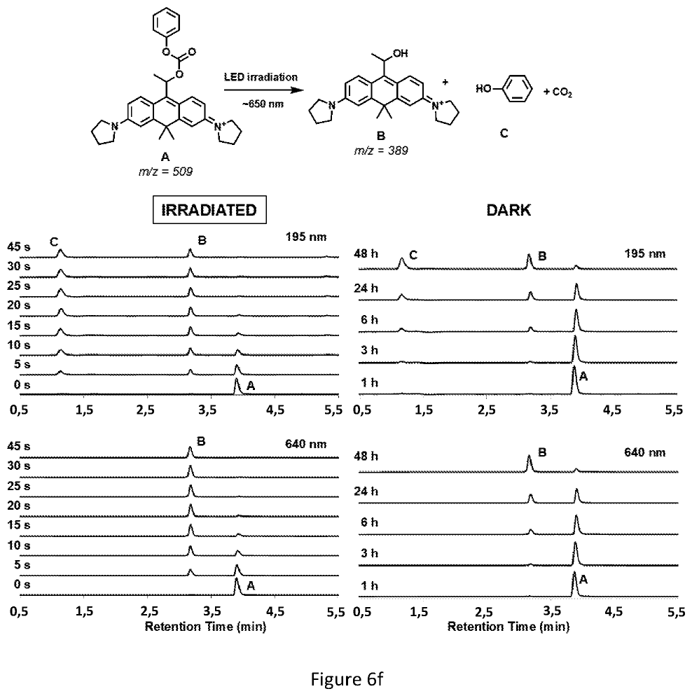

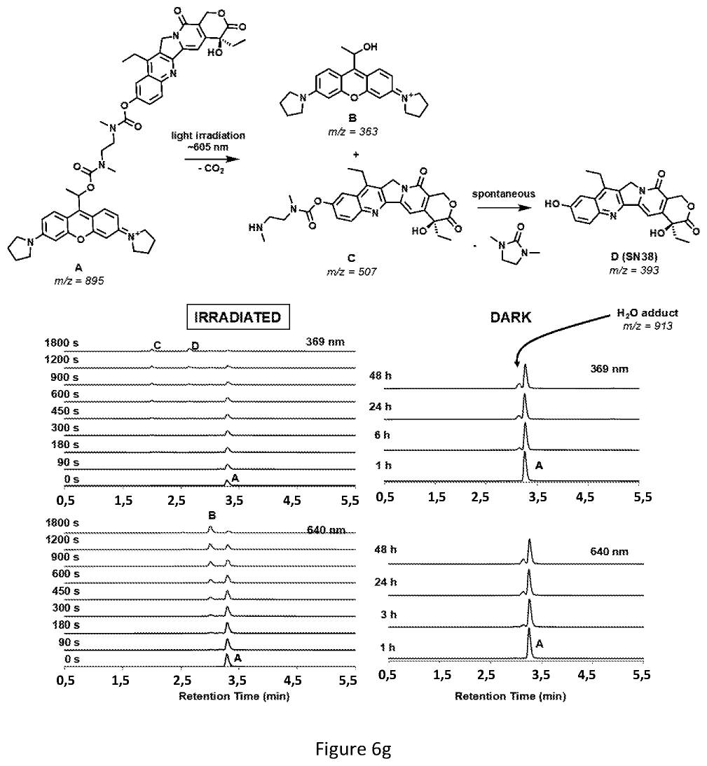

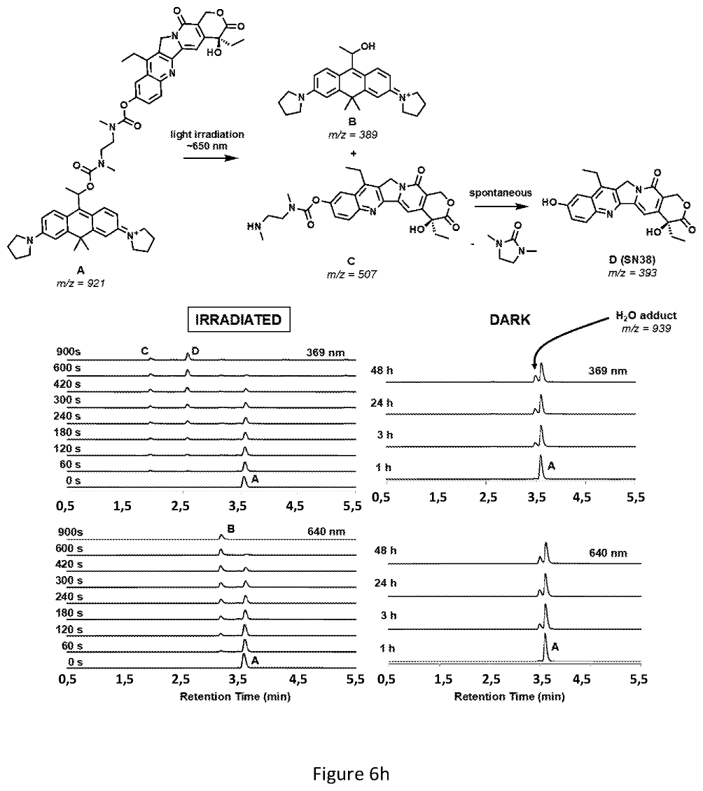

The photouncaging was evaluated by HPLC-MS using green (4 W input power, λₘₐₓ=549 nm half-width: 16 nm, output power: 72 mW), orange (4 W input power, λₘₐₓ=605 nm half-width: 10 nm, output power: 140 mW) and red (4 W input power, λₘₐₓ=658 nm half-width: 10 nm, for Q=CMe₂ output power: 210 mW) LED irradiation. For all compounds the experiments were performed in 90% water/MeCN mixtures as well as in 90% PBS/MeCN (0.1 mM concentration). In all cases, the irradiation led to the release of the model active agents with significant differences based on the photoremovable protecting group, cargo unit and the connecting chemical moiety as well as on the water content of the uncaging medium. Comparing the green light absorbing target conjugates, irradiation of 39 led to the release of 54% phenylacetic acid in 60 s (FIG. 6b), while 180 s (for 50%) was required for the same result in case of 41. Approximately 65% of phenylacetic acid was released after 180 s of irradiation with orange light from model target conjugate 41 (FIG. 6a). The current state of the art BODIPY derivative (for the structure, see the next section),^{20,21} released only 12% phenylacetic acid after irradiation for 1800 s. Contrary to the general expectations that increasing the wavelength diminishes photolysis rates, the uncaging of the redshifted Q=CMe₂ derivative 43 was also very effective, reaching >70% release of phenylacetic acid in 120 s after red light irradiation (FIG. 6c). The high efficiency of 43 as a red light-activatable photoremovable protecting group was also compared to a π-extended BODIPY derivative (substrate 7 in REF²¹, using methanol as the solvent in a concentration of 0.1 mM), the current state of the art in this region of visible light. It was found that with a similar cargo unit, an approximately 5-fold increase in the photolysis rate could be observed. Also, the BODIPY reference was insoluble in water rendering it unsuitable for biological applications.

Importantly, except in the case of compounds wherein the connecting chemical moiety was a carbonate moiety (i.e., compound 51, FIG. 6f), the target conjugates were stable in water as well as in PBS for 24 hours in the dark. The stability and uncaging rates of several leaving groups was also assessed. Comparing the most important functional groups (carboxylic acid, amine, phenol, alcohol) in potential active agents connected via the typical linkages in the target conjugates, the carbonate bond was the most labile (compound 51, >70% decomposition in 30 s with red light at 50% power) although the long-term dark stability was poor. None of the other model active agents were released in significant quantities, however, after 24-28 hours in the dark (for dark stability data see FIGS. 6a-6h, right side).

Using HPLC-MS, not only the appearance of the released model active agents could be monitored but also the products of photolysis were detectable. In all cases, the main product was the hydroxyethyl derivative also known as compound X wherein A=C⁺ and W═OH, as can be seen on FIGS. 6a-6h. In case of photoactivatable conjugate X2′-SN38, irradiation with red light led to the release of the linker-appended SN38 conjugate after 5-10 mins in PBS containing 10% MeCN (0.1 mM) with subsequent self-immolation, liberating free SN38 (FIG. 6h).

### Uncaging Quantum Yield (QY) Determination

The relative photochemical quantum yields of the uncaging reactions together with the degradation quantum yields were determined from the results of the quantitative HPLC-UV experiments. In all cases, green light irradiation was used since all target conjugates absorbed in this range as well as the reference BODIPY. The photochemical quantum yields were determined using the following equation:

$${Φ}_{u} = {Φ}_{u , ref} \cdot \frac{k \cdot c \cdot V \cdot {a}_{ref}}{{k}_{ref} \cdot {c}_{ref} \cdot {V}_{ref} \cdot a}$$

where
Φᵤ is the photochemical quantum yield of the sample
Φ_{u,ref} is the photochemical quantum yield of the reference
k is the obtained reaction rate constant of the photoreaction of the sample
k_{ref} is the obtained reaction rate constant of the photoreaction of the reference
c is the concentration of the sample
c_{ref} is the concentration of the reference
V is the volume of the sample
V_{ref} is the volume of the reference
a is the absorption correction factor of the sample
a_{ref} is the absorption correction factor of the reference

The absorption correction factors (a) were determined from the molar absorption coefficients and the green LED emission data obtained by the fluorimeter. We have calculated the transmittance (T) at each wavelength and calculated the efficiency of the illumination of each sample that resulted in the effectiveness of the irradiation. In other words, the transmittance at each wavelength together with the emission data of the light source can be used to calculate the percentage of absorbed photons according to the following equation:

$$a = {\sum }_{λ = {λ}_{min}}^{{λ}_{max}} [ ( 1 - T ( λ ) ) \frac{F ( λ )}{{\sum }_{λ = {λ}_{min}}^{{λ}_{max}} [ F ( λ ) ]} ]$$

where
T(λ) is the transmittance at each wavelength (determined from the molar absorption coefficients)
F(λ) is the LED emission at each wavelength
λₘᵢₙ, λₘₐₓ is the LED emission range.

The rate constants of the photoreactions were determined by considering the reactions monoexponential with an initial linear phase (up to 10-20% degradation). First, the concentration vs. HPLC-UV peak were determined for each cargo moieties. Then, in each case, a linear fitting of the time vs. released cargo concentration traces was performed that resulted in the rate constant k as determined from the fitted linear equation. The rate constants were determined for both the reference (with known Φᵤ) and the sample. For reference, a commonly known BODIPY derivative was used with a similar phenylacetic acid-derived cargo unit (substrate 7 from REF⁴⁰), Φᵤ=0.15%:

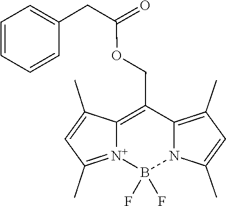

The calculated photochemical quantum yields together with uncaging cross-sections or quantum efficiencies (product of the molar absorption coefficient at a given wavelength, shown in parentheses and the photochemical quantum yield) are shown in Table 2. In the table, Φᵤ is the photochemical quantum yield of the release; Φ_{deg} is the photochemical quantum yield of degradation; ε×Φᵤ is the uncaging cross-section or quantum efficiency of the release at a given wavelength in parentheses.

TABLE 2
Uncaging quantum yields of the photocages

| compound | Φᵤ^{a} | Φ_{deg}^{b} | ε × Φᵤ (M⁻¹cm⁻¹)^{c} | |
|---|---|---|---|---|
| 39 | 4.7% | 6.2% | 1070 | (538 nm) |
| 41 | 1.5% | 1.9% | 1020 | (570 nm) |
| 43 | 1.7% | 2.2% | 1400 | (641 nm) |
| 49 | 0.92% | 1.7% | 690 | (637 nm) |
| 51 | 18% | 25% | 13 200 | (643 nm) |
| 45 | 1.6% | 2.6% | 1390 | (660 nm) |

| | | | |
|---|---|---|---|
| X1′-SN38^{d} | — | 0.15% | |
| X2′-SN38^{d} | — | 0.18% | — |

^{a}Uncaging quantum yield, calculated from active agent release
^{b}Degradation quantum yield, calculated from the disappearance of the target conjugate
^{c}Uncaging quantum efficiencies with the wavelength in parentheses
^{d}Exact release rate was not determined due to the complexity of the mixture

In general, the commonly accepted value of uncaging cross-section required for biological applications is above ^{˜}100 M⁻¹ cm⁻¹. ²As it is evident from Table 2, our PPGs exceed this value by an order of magnitude which is especially surprising considering their red-shifted absorption. It is generally expected that increasing the wavelength diminishes the photochemical quantum yield. Surprisingly, even the model target conjugates with the most redshifted absorption the uncaging cross-section reached 1400 M⁻¹ cm⁻¹, an unexpectedly high value.

Without wishing to be bound by any theory, it is believed that the surprisingly high efficiency of our PPGs is attributed to the low energy barrier of the heterolytic dissociation upon photoexcitation. The ^{˜}10% degradation quantum yield of the rhodol derivative 39 might be result of an energy barrier-free dissociation in the excited state facilitated by the lack of a positive charge on the chromophore scaffold.

### 5. Fluorescence Microscopy Experiments with the Target Conjugates X′-SN38

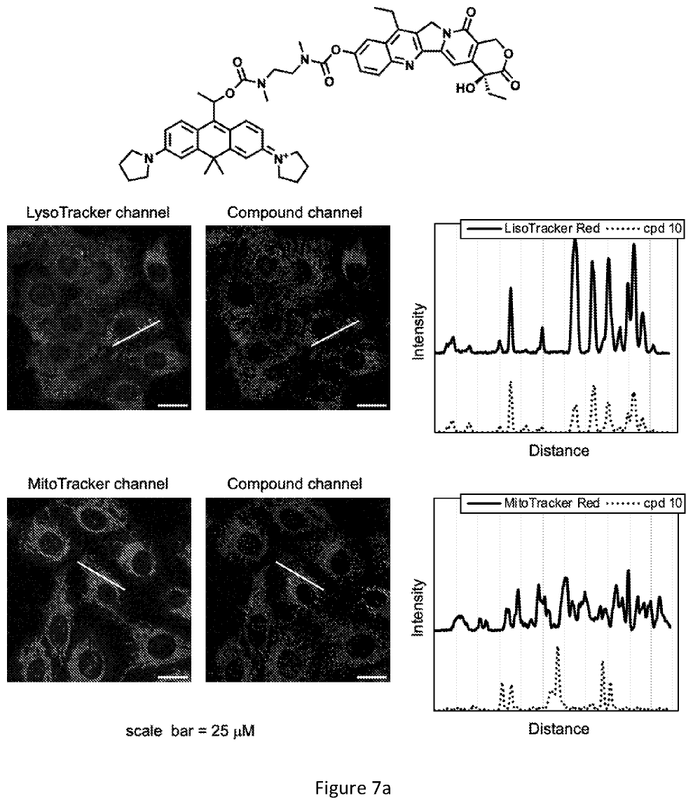

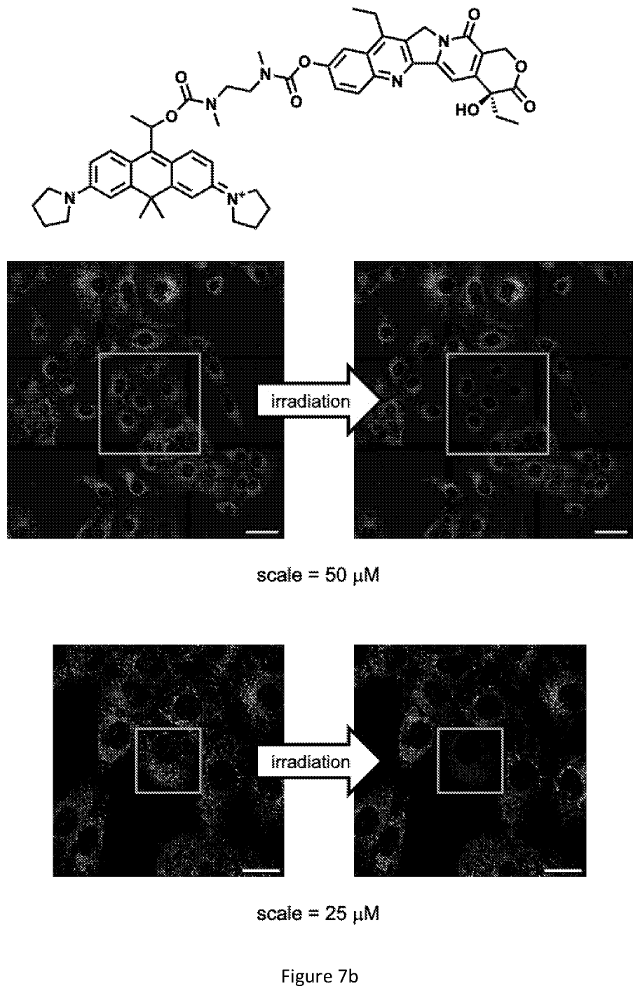

Due to their high fluorescence, the cell permeability of compounds X1′-SN38 and X2′-SN38 were tested in live SK—OV-3 (human ovarian carcinoma) cells using confocal microscopy. The colocalization was tested with commercially availabla MitoTrackers and LysoTrackers. In both cases, the compounds were cell permeable, an important feature of this invention. Interestingly, differing from most rhodamine derivatives that commonly localize in the mitochondria, fluorescence of compound X2′-SN38 (Q=CMe₂) was observed only in the lysosomes (in concentrations 100 nM and 1 uM, no wash conditions, FIG. 7a). In case of compound X1′-SN38 (Q=O), clear localization in the mitochondria was observed, concominant with results observable with rhodamine B. The spatial targeting of light irradiation was also tested using the built-in red laser of the instrument. Only a small fraction of the cells or simply one cell was irradiated with high intensity 638 nm laser light (for about 5-10 frames). The decrease of fluorescence (related to the photolysis reaction) was only observed in the irradiated area. See FIG. 7b for the images. Overall, these experiments serve as evidence for the cell permeability, biocompatibility and excellent ability of this invention to determine the localization of the photoactivatable conjugates via fluorescence microscopy.

### 6. Cytotoxicity Studies of the Target Conjugates X′-SN38

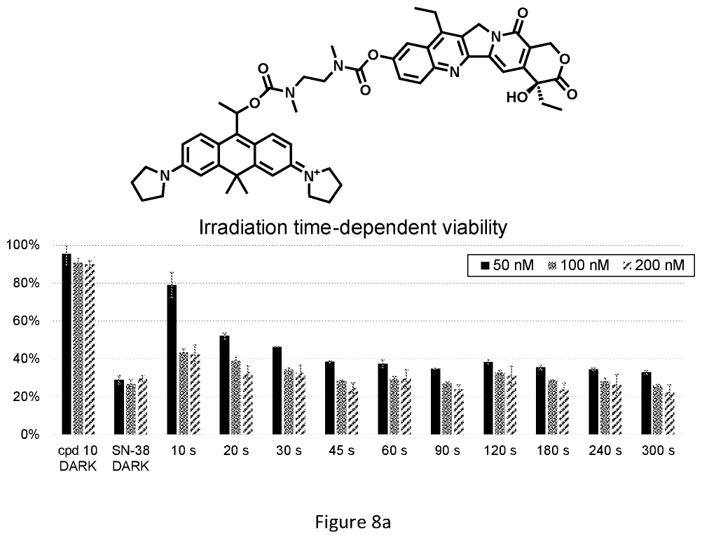

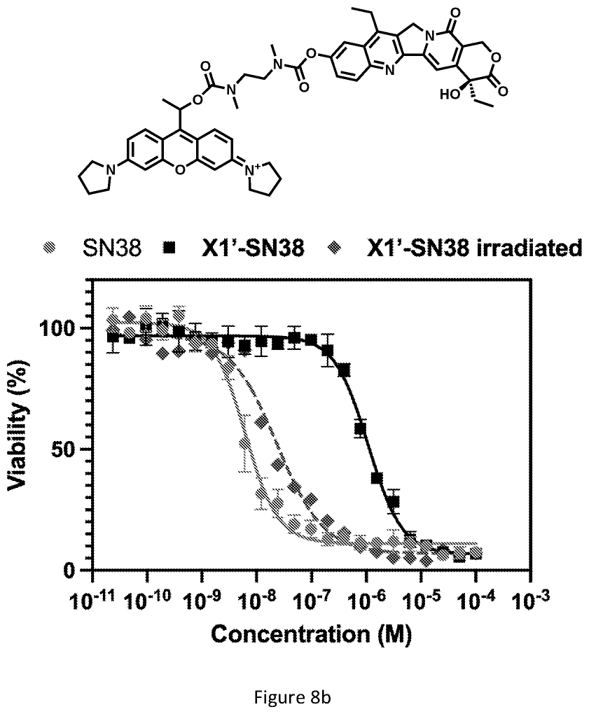

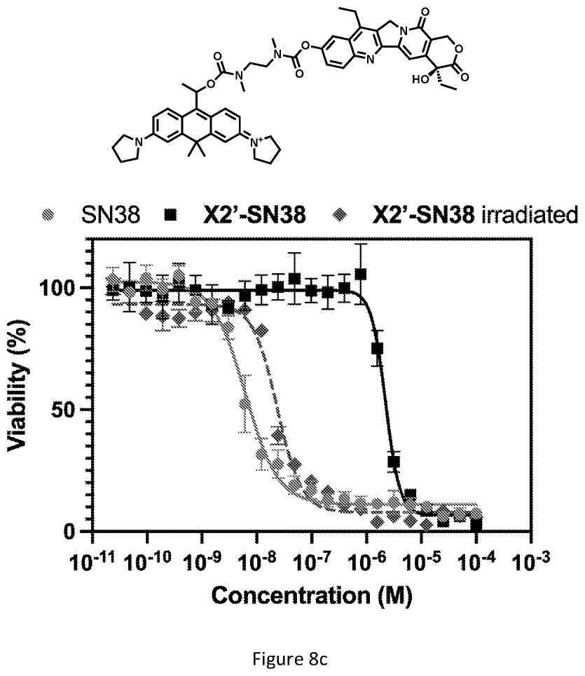

To determine phototoxic properties of compounds X1′-SN38 and X2′-SN38, standard MTT assays were performed on SK—OV-3 cells. To irradiate multiple specimens, an LED panel was custom-made for a 48-well plate consisting of 24 LEDs using green (4 W input power, λₘₐₓ=549 nm half-width: 16 nm, output power: 72 mW), orange (4 W input power, λₘₐₓ=605 nm half-width: 10 nm, output power: 140 mW) and red (4 W input power, λₘₐₓ=658 nm half-width: 10 nm, for Q=CMe₂ output power: 210 mW) LED irradiation. The board was specifically designed for the plates and offer water cooling to minimize heat shock to cells. Every second well can be irradiated with an individual LED. First, the cytotoxicity of the free active agent, SN38 was determined to be EC50 ^{˜}6 nM. Treatment of cells with the photoactivatable SN38 derivatives for 72 hours in the absence of light (without washing) produced moderate cytotoxic effects, sigificantly lowering the effectiveness of the active agent. This confirms that SN38 is a suitable candidate for light-activated prodrug applications. Brief irradiation of the cells with red light (60 s) after treatment with compound 10 for 90 min resulted in almost complete restoration of the activity of SN38 (EC50 for X2′-SN38 after irradiation: ^{˜}24 nM), providing an excellent photoindex of 92, almost an order of magnitude higher than current state of the art ruthenium-based PACT agents³⁰. Reducing the irradiation time down to 10 s still produced significant phototoxicity in higher concentrations (see FIG. 8a for compound X2′-SN38) even assuming incomplete uncaging as a result of the substantial dark stability of compound X2′-SN38. This effect is particularly important for further in vivo applications where lower irradiance can create significantly decreased uncaging yields.

For the complete uncaging of compound X1′-SN38, a higher irradiation time was required with orange LEDs, however, this compound still produced a significant phototoxic effect with a photoindex of 64.

TABLE 3
Concentration and light irradiation dependent cell
viabilities of the target conjugate X2′-SN38

| Irradiation time | 50 nM | 100 nM | 200 nM |
|---|---|---|---|
| X2′-SN38 DARK | 95% | 91% | 90% |
| SN38 DARK | 29% | 27% | 30% |
| 10 s | 79% | 44% | 42% |
| 20 s | 52% | 39% | 33% |
| 30 s | 46% | 35% | 33% |
| 45 s | 39% | 28% | 25% |
| 60 s | 38% | 30% | 30% |
| 90 s | 35% | 27% | 24% |
| 120 s | 39% | 33% | 31% |
| 180 s | 36% | 29% | 24% |
| 240 s | 35% | 28% | 26% |
| 300 s | 33% | 26% | 23% |

TABLE 4
Cytotoxicity of the photoactivatable SN38 derivatives

| | | | |
|---|---|---|---|
| | Dark EC50 | EC50 values | |
| Compound | values | after irradiation | Photoindex |

| | | | | |
|---|---|---|---|---|
| SN38 | 6.2 | nM | — | |
| X1′-SN38 | 4.0 | μM | 63 nM | 64 |
| (green) | | | | |
| X1′-SN38 | 1.1 | μM | 23 nM | 45 |
| (orange) | | | | |
| X2′-SN38 | 2.2 | μM | 24 nM | 95 |

For compound X2′-SN38, red light irradiation was applied (60 s), for compound X1′-SN38, green light irradiation was applied (15 min) as well as orange light (5 min).

### REFERENCES

(1) Welleman, I. M.; Hoorens, M. W. H.; Feringa, B. L.; Boersma, H. H.; Szymanski, W. Photoresponsive Molecular Tools for Emerging Applications of Light in Medicine. Chem. Sci. 2020. https://doi.org/10.1039/DOSC04187D.
(2) Klán, P.; Šolomek, T.; Bochet, C. G.; Blanc, A.; Givens, R.; Rubina, M.; Popik, V.; Kostikov, A.; Wirz, J. Photoremovable Protecting Groups in Chemistry and Biology: Reaction Mechanisms and Efficacy. Chem. Rev. 2013, 113 (1), 119-191. https://doi.org/10.1021/cr300177k.
(3) Ellis-Davies, G. C. R. Caged Compounds: Photorelease Technology for Control of Cellular Chemistry and Physiology. Nat. Methods 2007, 4 (8), 619-628. https://doi.org/10.1038/nmeth1072.
(4) Yu, H.; Li, J.; Wu, D.; Qiu, Z.; Zhang, Y. Chemistry and Biological Applications of Photo-Labile Organic Molecules. Chem. Soc. Rev. 2010, 39 (2), 464-473. https://doi.org/10.1039/b901255a.
(5) Gautier, A.; Gauron, C.; Volovitch, M.; Bensimon, D.; Jullien, L.; Vriz, S. How to Control Proteins with Light in Living Systems. Nat. Chem. Biol. 2014, 10 (7), 533-541. https://doi.org/10.1038/nchembio.1534.
(6) Hansen, M. J.; Velema, W. A.; Lerch, M. M.; Szymanski, W.; Feringa, B. L. Wavelength-Selective Cleavage of Photoprotecting Groups: Strategies and Applications in Dynamic Systems. Chem. Soc. Rev. 2015, 44 (11), 3358-3377. https://doi.org/10.1039/C5CS00118H.
(7) Sheehan, J. C.; Wilson, R. M.; Oxford, A. W. Photolysis of Methoxy-Substituted Benzoin Esters. Photosensitive Protecting Group for Carboxylic Acids. J. Am. Chem. Soc. 1971, 93 (26), 7222-7228. https://doi.org/10.1021/Ja00755a017.
(8) Park, C.-H.; Givens, R. S. New Photoactivated Protecting Groups. 6. p-Hydroxyphenacyl: A Phototrigger for Chemical and Biochemical Probes 1, 2. J. Am. Chem. Soc. 1997, 119 (10), 2453-2463. https://doi.org/10.1021/ja9635589.
(9) Olson, J. P.; Banghart, M. R.; Sabatini, B. L.; Ellis-Davies, G. C. R. Spectral Evolution of a Photochemical Protecting Group for Orthogonal Two-Color Uncaging with Visible Light. J. Am. Chem. Soc. 2013, 135 (42), 15948-15954. https://doi.org/10.1021/ja408225k.
(10) Dunkel, P.; Ilaš, J. Targeted Cancer Therapy Using Compounds Activated by Light. Cancers (Basel). 2021, 13 (13), 3237. https://doi.org/10.3390/cancers13133237.
(11) Agarwal, H. K.; Janicek, R.; Chi, S. H.; Perry, J. W.; Niggli, E.; Ellis-Davies, G. C. R. Calcium Uncaging with Visible Light. J. Am. Chem. Soc. 2016, 138 (11), 3687-3693. https://doi.org/10.1021/jacs.5b11606.
(12) Farley, S.; Laguerre, A.; Schultz, C. Caged Lipids for Subcellular Manipulation. Curr. Opin. Chem. Biol. 2021, 65, 42-48. https://doi.org/10.1016/j.cbpa.2021.04.012.
(13) Mangubat-Medina, A. E.; Ball, Z. T. Triggering Biological Processes: Methods and Applications of Photocaged Peptides and Proteins. Chem. Soc. Rev. 2021, 10403-10421. https://doi.org/10.1039/dOcs01434f.
(14) Vickerman, B. M.; Zywot, E. M.; Tarrant, T. K.; Lawrence, D. S. Taking Phototherapeutics from Concept to Clinical Launch. Nat. Rev. Chem. 2021, 5 (11), 816-834. https://doi.org/10.1038/s41570-021-00326-w.
(15) Weinstain, R.; Slanina, T.; Kand, D.; Klán, P. Visible-to-NIR-Light Activated Release: From Small Molecules to Nanomaterials. Chem. Rev. 2020, 120 (24), 13135-13272. https://doi.org/10.1021/acs.chemrev.0c00663.
(16) Sharma, M.; Friedman, S. H. The Issue of Tissue: Approaches and Challenges to the Light Control of Drug Activity. ChemPhotoChem 2021, cptc.202100001. https://doi.org/10.1002/cptc.202100001.
(17) Bojtár, M.; Kormos, A.; Kis-Petik, K.; Kellermayer, M.; Kele, P. Green-Light Activatable, Water-Soluble Red-Shifted Coumarin Photocages. Org. Lett. 2019, 21 (23), 9410-9414. https://doi.org/10.1021/acs.orglett.9b03624.
(18) Bojtár, M.; Németh, K.; Domahidy, F.; Knorr, G.; Verkman, A.; Kállay, M.; Kele, P. Conditionally Activatable Visible-Light Photocages. J. Am. Chem. Soc. 2020, 142 (35), 15164-15171. https://doi.org/10.1021/jacs.0c07508.
(19) Rubinstein, N.; Liu, P.; Miller, E. W.; Weinstain, R. Meso-Methylhydroxy BODIPY: A Scaffold for Photo-Labile Protecting Groups. Chem. Commun. 2015, 51 (29), 6369-6372. https://doi.org/10.1039/C5CC00550G.
(20) Goswami, P. P.; Syed, A.; Beck, C. L.; Albright, T. R.; Mahoney, K. M.; Unash, R.; Smith, E. A.; Winter, A. H. BODIPY-Derived Photoremovable Protecting Groups Unmasked with Green Light. J. Am. Chem. Soc. 2015, 137 (11), 3783-3786. https://doi.org/10.1021/jacs.5b01297.
(21) Peterson, J. A.; Wijesooriya, C.; Gehrmann, E. J.; Mahoney, K. M.; Goswami, P. P.; Albright, T. R.; Syed, A.; Dutton, A. S.; Smith, E. A.; Winter, A. H. Family of BODIPY Photocages Cleaved by Single Photons of Visible/Near-Infrared Light. J. Am. Chem. Soc. 2018, 140 (23), 7343-7346. https://doi.org/10.1021/jacs.8b04040.
(22) Nani, R. R.; Gorka, A. P.; Nagaya, T.; Kobayashi, H.; Schnermann, M. J. Near-IR Light-Mediated Cleavage of Antibody-Drug Conjugates Using Cyanine Photocages. Angew. Chem. Int. Ed. 2015, 54 (46), 13635-13638. https://doi.org/10.1002/anie.201507391.
(23) Yamamoto, T.; Caldwell, D. R.; Gandioso, A.; Schnermann, M. J. A Cyanine Photooxidation/β-Elimination Sequence Enables Near-infrared Uncaging of Aryl Amine Payloads. Photochem. Photobiol. 2019, 95 (4), php.13090. https://doi.org/10.1111/php.13090.
(24) Gorka, A. P.; Nani, R. R.; Schnermann, M. J. Harnessing Cyanine Reactivity for Optical Imaging and Drug Delivery. Acc. Chem. Res. 2018, 51 (12), 3226-3235. https://doi.org/10.1021/acs.accounts.8b00384.
(25) Toupin, N.; Steinke, S. J.; Nadella, S.; Li, A.; Rohrabaugh, T. N.; Samuels, E. R.; Turro, C.; Sevrioukova, I. F.; Kodanko, J. J. Photosensitive Ru(II) Complexes as Inhibitors of the Major Human Drug Metabolizing Enzyme CYP3A4. J. Am. Chem. Soc. 2021, 143 (24), 9191-9205. https://doi.org/10.1021/jacs.1c04155.
(26) Jakubaszek, M.; Goud, B.; Ferrari, S.; Gasser, G. Mechanisms of Action of Ru(li) Polypyridyl Complexes in Living Cells upon Light Irradiation. Chem. Commun. (Camb). 2018, 54 (93), 13040-13059. https://doi.org/10.1039/c8cc05928d.
(27) Shrestha, P.; Dissanayake, K. C.; Gehrmann, E. J.; Wijesooriya, C. S.; Mukhopadhyay, A.; Smith, E. A.; Winter, A. H. Efficient Far-Red/Near-IR Absorbing BODIPY Photocages by Blocking Unproductive Conical Intersections. J. Am. Chem. Soc. 2020, 142 (36), 15505-15512. https://doi.org/10.1021/jacs.0c07139.
(28) Janeková, H.; Russo, M.; Ziegler, U.; Štacko, P. Photouncaging of Carboxylic Acids from Cyanine Dyes with Near-Infrared Light**. Angew. Chem. Int. Ed. 2022, 61 (33). https://doi.org/10.1002/anie.202204391.
(29) Bonnet, S. Why Develop Photoactivated Chemotherapy? Dalt. Trans. 2018, 47 (31), 10330-10343. https://doi.org/10.1039/C8DT01585F.
(30) van Rixel, V. H. S.; Ramu, V.; Auyeung, A. B.; Beztsinna, N.; Leger, D. Y.; Lameijer, L. N.; Hilt, S. T.; Le Dévédec, S. E.; Yildiz, T.; Betancourt, T.; Gildner, M. B.; Hudnall, T. W.; Sol, V.; Liagre, B.; Kornienko, A.; Bonnet, S. Photo-Uncaging of a Microtubule-Targeted Rigidin Analogue in Hypoxic Cancer Cells and in a Xenograft Mouse Model. J. Am. Chem. Soc. 2019, 141 (46), 18444-18454. https://doi.org/10.1021/jacs.9b07225.
(31) Šebej, P.; Wintner, J.; Müller, P.; Slanina, T.; Al Anshori, J.; Antony, L. A. P.; Klán, P.; Wirz, J. Fluorescein Analogues as Photoremovable Protecting Groups Absorbing at ˜−520 Nm. J. Org. Chem. 2013, 78 (5), 1833-1843. https://doi.org/10.1021/jo301455n.
(32) Šolomek, T.; Wirz, J.; Klán, P. Searching for Improved Photoreleasing Abilities of Organic Molecules. Acc. Chem. Res. 2015, 48 (12), 3064-3072. https://doi.org/10.1021/acs.accounts.5b00400.
(33) Kitamura, K.; Kawaguchi, M.; leda, N.; Miyata, N.; Nakagawa, H. Visible Light-Controlled Nitric Oxide Release from Hindered Nitrobenzene Derivatives for Specific Modulation of Mitochondrial Dynamics. ACS Chem. Biol. 2016, 11 (5), 1271-1278. https://doi.org/10.1021/acschembio.5b00962.
(34) Alouane, A.; Labruère, R.; Le Saux, T.; Schmidt, F.; Jullien, L. Self-Immolative Spacers: Kinetic Aspects, Structure-Property Relationships, and Applications. Angew. Chem. Int. Ed. 2015, 54 (26), 7492-7509. https://doi.org/10.1002/anie.201500088.
(35) Wu, L.; Burgess, K. Synthesis and Spectroscopic Properties of Rosamines with Cyclic Amine Substituents. J. Org. Chem. 2008, 73 (22), 8711-8718. https://doi.org/10.1021/jo800902j.
(36) Grimm, J. B.; Sung, A. J.; Legant, W. R.; Hulamm, P.; Matlosz, S. M.; Betzig, E.; Lavis, L. D. Carbofluoresceins and Carborhodamines as Scaffolds for High-Contrast Fluorogenic Probes. ACS Chem. Biol. 2013, 8 (6), 1303-1310. https://doi.org/10.1021/cb4000822.
(37) Pastierik, T.; Šebej, P.; Medalová, J.; Štacko, P.; Klán, P. Near-Infrared Fluorescent 9-Phenylethynylpyronin Analogues for Bioimaging. J. Org. Chem. 2014, 79 (8), 3374-3382. https://doi.org/10.1021/jo500140y.
(38) Bachman, J. L.; Pavlich, C. I.; Boley, A. J.; Marcotte, E. M.; Anslyn, E. V. Synthesis of Carboxy ATTO 647N Using Redox Cycling for Xanthone Access. Org. Lett. 2020, 22 (2), 381-385. https://doi.org/10.1021/acs.orglett.9b03981.
(39) Pal, S.; Mukherjee, M.; Sen, B.; Mandal, S. K.; Lohar, S.; Chattopadhyay, P.; Dhara, K. A New Fluorogenic Probe for the Selective Detection of Carbon Monoxide in Aqueous Medium Based on Pd(0) Mediated Reaction. Chem. Commun. 2015, 51 (21), 4410-4413. https://doi.org/10.1039/C5CC00902B.
(40) Slanina, T.; Shrestha, P.; Palao, E.; Kand, D.; Peterson, J. A.; Dutton, A. S.; Rubinstein, N.; Weinstain, R.; Winter, A. H.; Kln, P. In Search of the Perfect Photocage: Structure-Reactivity Relationships in Meso-Methyl BODIPY Photoremovable Protecting Groups. J. Am. Chem. Soc. 2017, 139 (42), 15168-15175. https://doi.org/10.1021/Jacs.7b08532.

1. Compound represented by formula (Xa), 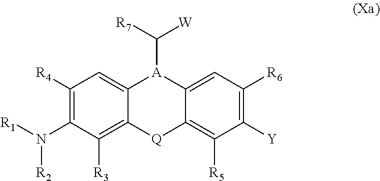
wherein
A is either C⁺ or CH,
when A is C⁺, the compound comprises a counter anion;
R₁ and R₂ are each selected independently from hydrogen, C₁-C₆ alkyl, C₃-C₆ cycloalkyl, C₂-C₆ alkenyl, C₆-C₁₀ aryl; or
R₁ and R₂, together with the nitrogen atom, to which they are attached, form a 3-10-membered heterocycloalkyl ring containing said N atom, and optionally containing a further heteroatom selected from N, O and S;
Y is selected from NR₈R₉ or OR₁₀, wherein
R₈ and R₉ are each independently selected from hydrogen, C₁-C₆ alkyl, C₃-C₆ cycloalkyl, C₂-C₆ alkenyl and C₆-C₁₀ aryl; or
R₈ and R₉, together with the nitrogen atom, to which they are attached, form a 3-10-membered heterocycloalkyl ring containing said N atom, and optionally containing a further heteroatom selected from N, O and S;
R₁₀ is selected from hydrogen and OH-protecting group selected from C₁-C₆ alkoxymethyl, C₁-C₆ alkyl, C₃-C₆ cycloalkyl, C₂-C₆ alkenyl, aryl, arylmethyl, acetyl, benzoyl, tetrahydropyranyl, tetrahydrofuranyl, tetrahydrothiofuranyl, and C₁-C₆ alkyl or aryl-substituted silyl;
R₃, R₄, R₅, R₆ are each selected independently from H, halogen, C₁-C₆ alkyl, C₃-C₆ cycloalkyl or C₃-C₆ cycloalkenyl, or
R₃ and R₂, R₄ and R₁, R₅ and R₉, and/or R₆ and R₈ together with the intervening atoms, may form a 5-7-membered non aromatic, saturated or unsaturated heterocycle containing a N atom, and optionally containing a further heteroatom selected from N, O and S,
R₇ is C₁-C₆ alkyl, C₂-C₆ alkenyl, C₃-C₆ cycloalkyl, C₆-C₁₀ aryl or (C₆-C₁₀ aryl)(C₁-C₆ alkyl);
Q is selected from NH, NR₁₁, O, C(R₁₁)₂, Si(R₁₁)₂, P=O(Ph) or P=O(OPh) or P═O(O⁻), wherein
each R₁₁ is independently selected from C₁-C₆ alkyl, C₃-C₆ cycloalkyl, C₂-C₆ alkenyl, C₆-C₁₀ aryl and (C₆-C₁₀ aryl)(C₁-C₆ alkyl);
W is either
OH,
halogen, or
an oxycarbonyl linker precursor moiety or a self-immolative linker precursor moiety,
wherein
in the meaning of R₁ to R₁₁ each instance of alkyl and alkenyl is optionally substituted with one or more substituent(s) independently selected from the group of halogen, hydroxyl and CF₃,
in the meaning of R₁ to R₁₁ each instance of cycloalkyl is optionally substituted with one or more substituent(s) independently selected from the group of C₁-C₄ alkyl, halogen, hydroxyl and CF₃,
the 3-10-membered heterocycloalkyl formed by NR₁R₂ or NR₈R₉ is optionally substituted with one or more substituent(s) independently selected from the group of C₁-C₄ alkyl, halogen, hydroxyl and CF₃,
the 5-7-membered non aromatic, saturated or unsaturated heterocycle formed by R₃ and R₂, R₄ and R₁, R₅ and R₉, and/or R₆ and R₈ together with the intervening atoms is optionally substituted with one or more substituent(s) independently selected from the group of C₁-C₄ alkyl, halogen, hydroxyl and CF₃,
in the meaning of R₁, R₂, R₇-R₉ and R₁₁ each instance of aryl is optionally substituted with one or more substituent(s) independently selected from the group of C₁-C₄ alkyl, halogen and CF₃,
in any protonation state thereof, or salts thereof.

2. The compound of claim 1, wherein
R₁ and R₂ are each selected independently from hydrogen and C₁-C₆ alkyl; or
R₁ and R₂, together with the nitrogen atom, to which they are attached, form a 4-6-membered heterocycloalkyl ring containing said N atom, and optionally containing a further heteroatom selected from N and O;
Y is NR₈R₉, wherein
R₈ and R₉ are each independently selected from hydrogen and C₁-C₆ alkyl; or
R₈ and R₉, together with the nitrogen atom, to which they are attached, form a 4-6-membered heterocycloalkyl ring containing said N atom, and optionally containing a further heteroatom selected from N and O;
R₃, R₄, R₅, R₆ are each selected independently from H and C₁-C₆ alkyl; and
R₃ and R₂, R₄ and R₁, R₅ and R₉, and/or R₆ and R₈ together with the intervening atoms, may form a 6-membered non aromatic, saturated or unsaturated heterocycle containing a N atom and optionally containing a further heteroatom selected from N and O, said heterocycle being optionally substituted with 1-6 C₁-C₄ alkyl;
in any protonation state thereof, or salts thereof.

3. The compound of claim 1, wherein
R₁ and R₂ are each selected independently from hydrogen and C₁-C₆ alkyl; or
R₁ and R₂, together with the nitrogen atom, to which they are attached, form a 4-6-membered heterocycloalkyl ring containing said N atom, and optionally containing a further heteroatom selected from N and O;
Y is OR₁₀, wherein
R₁₀ is selected from hydrogen and OH-protecting group selected from C₁-C₆ alkoxymethyl, C₁-C₆ alkyl, C₃-C₆ cycloalkyl, C₂-C₆ alkenyl, aryl, arylmethyl, acetyl, benzoyl, tetrahydropyranyl, tetrahydrofuranyl, tetrahydrothiofuranyl, and C₁-C₆ alkyl or aryl-substituted silyl;
R₃, R₄, R₅, R₆ are each selected independently from H and C₁-C₆ alkyl; and
R₃ and R₂, and/or R₄ and R₁ together with the intervening atoms, may form a 6-membered non aromatic, saturated or unsaturated heterocycle containing a N atom and optionally containing a further heteroatom selected from N and O, said heterocycle being optionally substituted with 1-6 C₁-C₄ alkyl;
in any protonation state thereof, or salts thereof.

4. The compound of claim 3, wherein
Y is OR₁₀, wherein R₁₀ is hydrogen; and
A is C⁺
in any protonation state thereof, or salts thereof.

5. The compound of claim 4 in its deprotonated state, represented by formula (Xd) 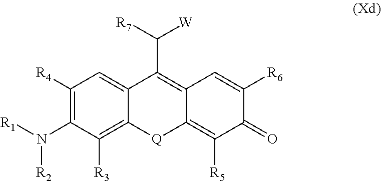
or salts thereof.

6. The compound of claim 1, wherein Q is selected from O and C(R₁₁)₂, wherein
R₁₁ is selected from C₁-C₆ alkyl groups;
or salts thereof.

7. The compound of claim 1, wherein R₇ is C₁-C₆ alkyl, C₃-C₆ cycloalkyl or (C₆-C₁₀ aryl)(C₁-C₆ alkyl);
or salts thereof.

8. The compound of claim 1, wherein W is either
OH, or
halogen,
or salts thereof.

9. The compound of claim 1, wherein W is either
an oxycarbonyl linker precursor moiety selected from: 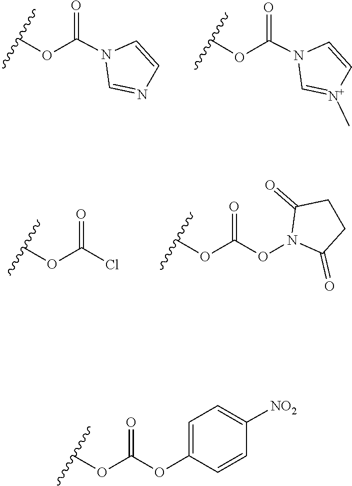
or a self-immolative linker precursor moiety selected from: 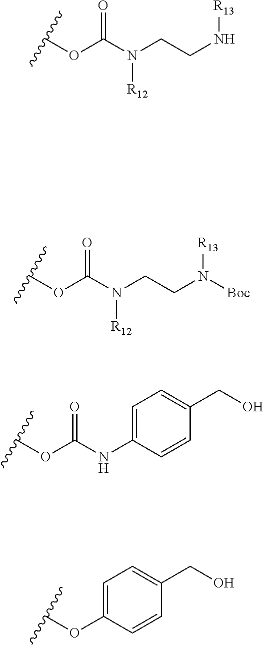
wherein R₁₂ and R₁₃ are independently selected from hydrogen, C₁-C₆ alkyl, C₃-C₆ cycloalkyl, C₂-C₆ alkenyl; or R₁₂ or R₁₃, together with the nitrogen atom to which they are attached and with one or both of the carbon atoms of the ethylene moiety, form a 4-6 membered heterocycloalkyl containing said N atom, and optionally containing a further heteroatom selected from N, O and S; and
the wavy line ( [symbol] ) represents the attachment point to the rest of the molecule, or salts thereof.

10. The compound of claim 1, wherein when A is C⁺ and the counter anion is selected from Cl⁻, Br⁻, I⁻, HCOO⁻, AcO⁻, CF₃COO⁻, BF₄⁻, PF₆⁻, methanesulfonate, sulfate, phosphate or citrate.

11. The compound of any of claim 1, having one of the following formulas: 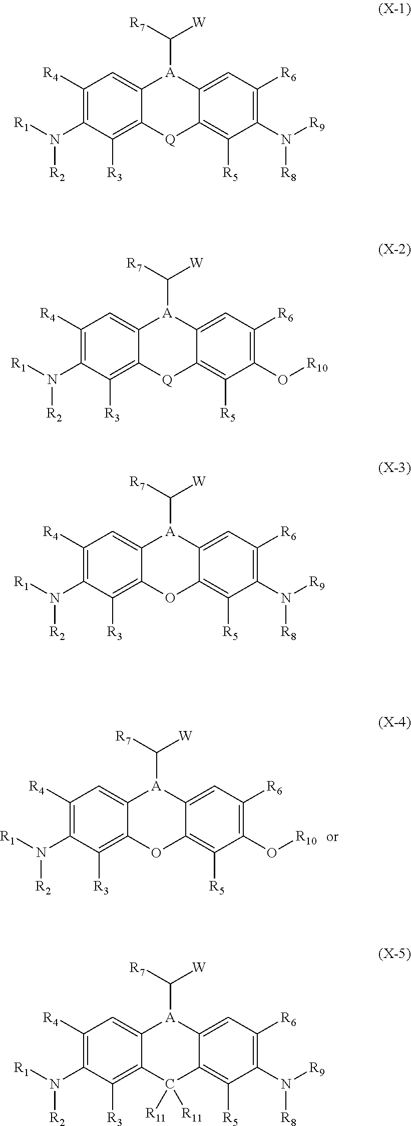
wherein the substituents are as defined in claim 1.

12. The compound of claim 1, having one of the following formulas:

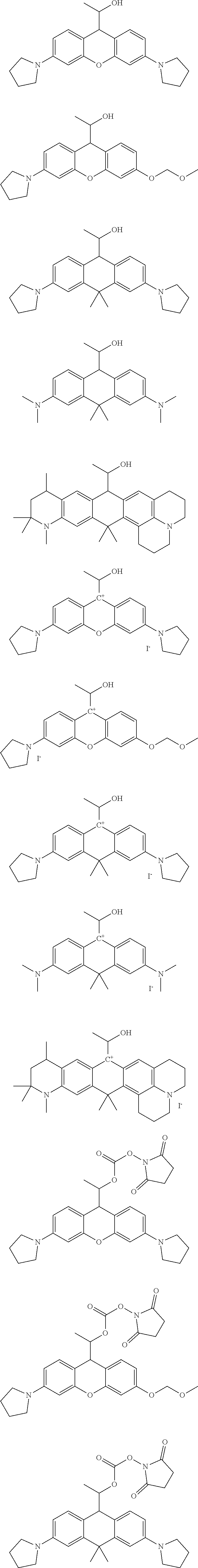

13. A precursor for a photoremovable protecting group of formulas (Xa′) or (Xd′): 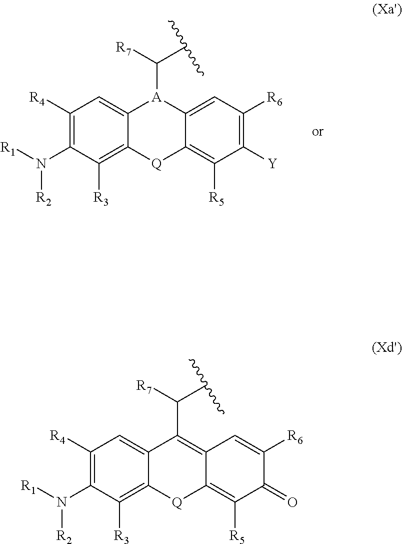
wherein A is C⁺ and the group comprises a counter anion,
Y, Q, and R₁ to R₇ are as defined in claim 1;
comprising the compound of claim 1.

14. A photoactivatable conjugate comprising a photoremovable protecting group derived from a compound of claim 1, represented by formulas (Xa′) or (Xd′): 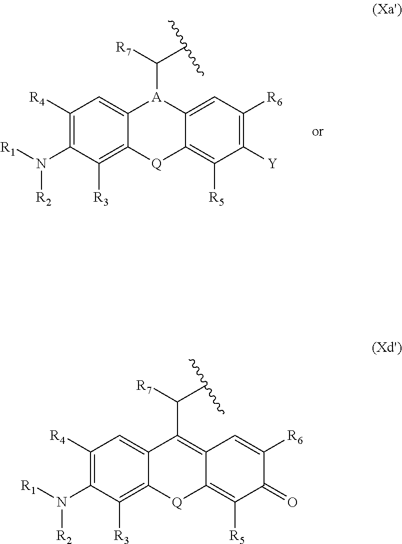
wherein A is C⁺ and the compound comprises a counter anion,
Y, Q and R₁ to R₇ are as defined in claim 1, and
the wavy line ( [symbol] ) represents the attachment point to the rest of the molecule,
or a pharmaceutically acceptable salt thereof.

15. A photoactivatable conjugate or a pharmaceutically acceptable salt thereof according to claim 14, which is represented by formula X′-L-Z′, wherein
X′ has the following formula: 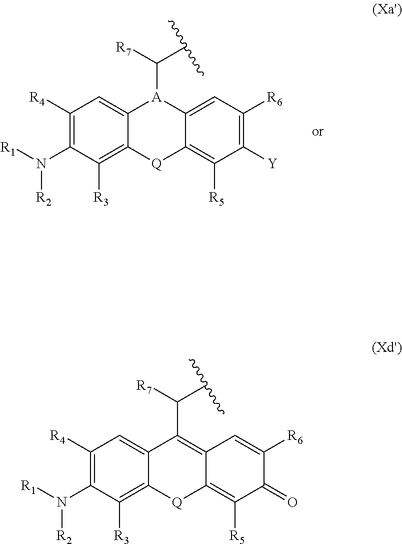
wherein A is C⁺ and the compound comprises a counter anion,
Y, Q, and R₁ to R₇ are as defined in claim 14,
the wavy line ( [symbol] ) represents the attachment point to L,
L is a covalent bond or a linker, having one of the following formulas: 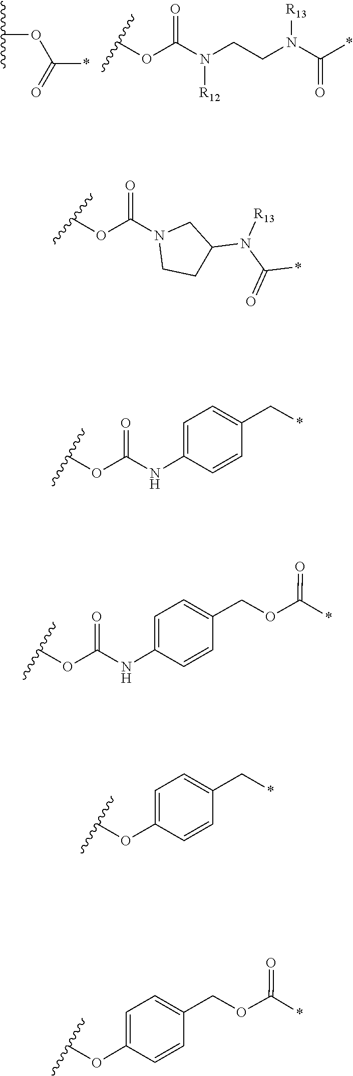
wherein,
R₁₂ and R₁₃ are independently selected from hydrogen, C₁-C₆ alkyl, C₃-C₆ cycloalkyl, C₂-C₆ alkenyl; or R₁₂ or R₁₃, together with the nitrogen atom to which they are attached and with one or both of the carbon atoms of the ethylene moiety, form a 4-6 membered heterocycloalkyl containing said N atom, and optionally containing a further heteroatom selected from N, O and S;
the wavy line ( [symbol] ) represents the attachment point to X′ and the asterisk
( [symbol] ) represents the attachment point to Z′;
Z′ is selected from a moiety derived from an active pharmaceutical agent by chemical derivatization.

16. The photoactivatable conjugate or a pharmaceutically acceptable salt thereof according to claim 15, wherein Z′ is a moiety derived from a cytotoxic chemotherapeutic agent or inhibitor used for the destruction of cancer cells.

17. The photoactivatable conjugate or a pharmaceutically acceptable salt thereof according to claim 15 wherein Z′ is a moiety derived from nucleoside analogs such as cytarabine or fluorouracil, antifolates such as methotrexate, topoisomerase inhibitors such as camptothecin, irinotecan or SN38, anthracyclines such as doxorubicin, daunomycin or mitoxantrone, taxanes such as paclitaxel or docetaxel, vinca alcaloids such as vincristine and vinblastine, alkylating agents such as chlorambucil, melphalan or cyclophosphamide, platinum compounds such as carboplatin or cisplatin, or targeted antineoplastic agents such as tyrosine kinase inhibitors ibrutinib or dasatinib, histone deacetylase inhibitor vorinostat, or other agents such as methylprednisolone, retinoids, thalidomide.

18. The photoactivatable conjugate or a pharmaceutically acceptable salt thereof according to claim 15, selected from: 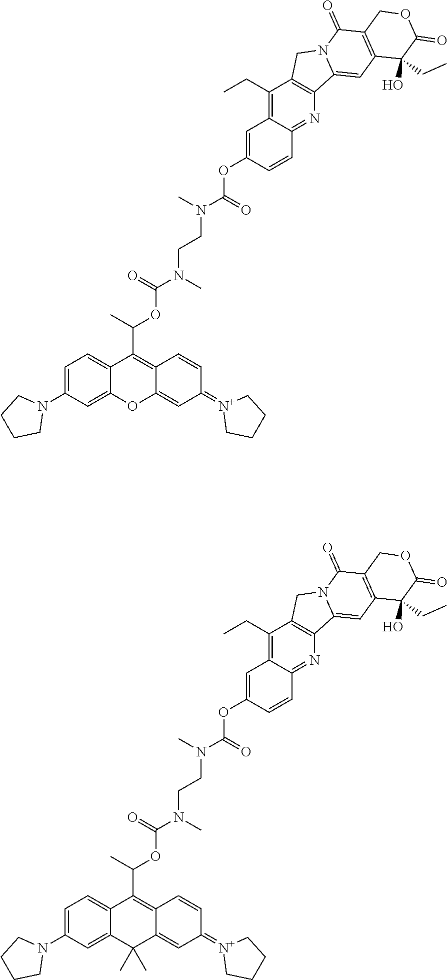 [SMILES: CCc1c2c(nc3ccc(OC(=O)N(C)CCN(C)C(=O)OC(C)c4c5ccc(=[N+]6CCCC6)cc-5oc5cc(N6CCCC6)ccc45)cc13)-c1cc3c(c(=O)n1C2)COC(=O)[C@]3(O)CC ; CCc1c2c(nc3ccc(OC(=O)N(C)CCN(C)C(=O)OC(C)C4=C5C=CC(=[N+]6CCCC6)C=C5C(C)(C)c5cc(N6CCCC6)ccc54)cc13)-c1cc3c(c(=O)n1C2)COC(=O)[C@]3(O)CC] 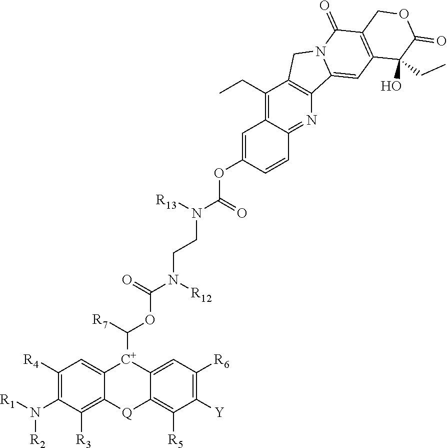
wherein the compound comprises a counter anion, Y, Q, and R₁ to R₇ and R₁₂ and R₁₃ are defined in claim 15,
or a pharmaceutically acceptable salt thereof.

19. Pharmaceutical composition comprising the photoactivatable conjugate or a pharmaceutically acceptable salt thereof according to claim 14 together with one or more pharmaceutically acceptable excipient.

20. A method of photoactivated therapy, which comprises administering a photoactivatable conjugate or a pharmaceutically acceptable salt thereof according to claim 14 to a subject in need thereof, followed by irradiation with visible light having a wavelength >550 nm in a spatiotemporally controlled fashion in the subject.

21. The method according to claim 20, wherein the photoactivated therapy comprises the treatment of a disorder of a subject, which disorder is highly localizable in the body of the subject, and the locus of the disorder is irradiated using the visible light with a wavelength of >550 nm.

22. The method according to claim 21, wherein the disorder, which is highly localizable in the body of the subject, is cancer.

23. A method for photoreleasing an active agent Z from a photoactivatable conjugate of a formula X′-L-Z′ according to claim 15, comprising irradiating a biological sample selected from cell cultures or xenograft tumor models containing X′-L-Z′ with a wavelength >550 nm so as to release the active agent Z, wherein
X′, L and Z′ is as defined in claim 15 and Z is the parent compound of Z′.

24. Process for the preparation of a compound according to claim 1, including:
a) reacting a compound of formula (1): 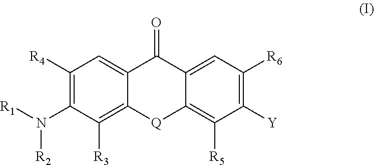
with a Grignard reagent of formula R₇—CH₂MgHal, wherein Hal is halogen, or with a compound of formula R₇CH₂Li, and converting the resulting adduct to an olefin derivative using first a proton source and then subsequent deprotonation using a strong base, to obtain a compound of formula (II): 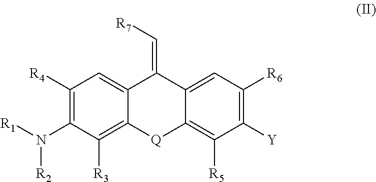
subjecting the compound of formula (II) to hydroboration-oxidation reaction for the anti-Markovnikov addition of a hydroxyl group and a hydrogen to the olefin bond to obtain a compound of formula (Xa) wherein A=CH and W═OH: 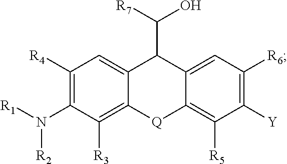
or
b) reacting a compound of formula (I): 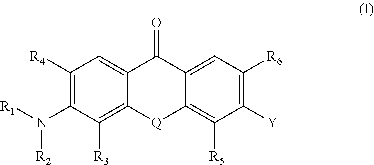
with a deprotonated vinyl ether derivative and hydrolysing the resulting vinyl ether appended compound to obtain a compound of formula (III): 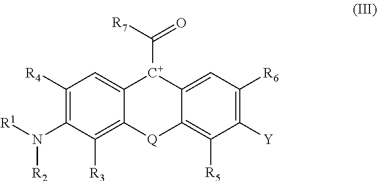
reducing the compound of formula (III), to obtain a compound of formula (Xa) wherein A=CH and W═OH: 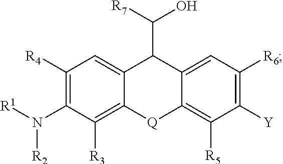
or
c) reacting a compound of formula (I): 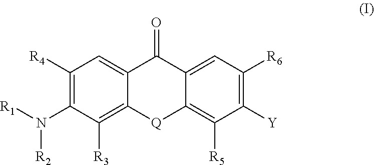
with a Grignard reagent of formula R₇—CH₂MgHal, wherein Hal is halogen, or with a compound of formula R₇CH₂Li, and converting the resulting adduct to an olefin derivative using first a proton source and then subsequent deprotonation using a strong base, to obtain a compound of formula (II): 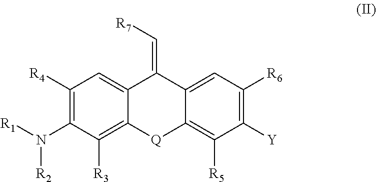
reacting the compound of formula (II) with N-halogenosuccinimide in the presence of water for the addition of HO-Hal to the olefin bond, then eliminating a hydroxide ion, to obtain a compound of formula (Xa) wherein A=C⁺ and the compound comprises a counter anion, designated as “Anion⁻”, and W=halogen: 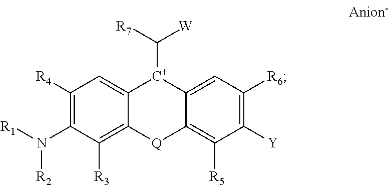
and optionally
d) converting the compounds of formula (Xa) wherein W═OH to compounds of formula (Xa) wherein W=oxycarbonyl linker precursor moiety using carbonic acid-derived activation reagents,
and optionally
e) converting the compounds of formula (Xa) wherein W=oxycarbonyl linker precursor moiety to compounds of formula (Xa) wherein W=self-immolative linker precursor moiety by reaction with a self-immolative linker precursor compound (e.g. with either N-Boc-N,N′-dimethylethylenediamine in the presence of a base, or 4-hydroxymethylaniline in the presence of a base and 1-hydroxybenzotriazole),
and optionally
f) oxidizing the compounds of formula (Xa) wherein A=CH using chloranil and subsequent anion replacement to compounds of formula (Xa) wherein A=C⁺,
wherein the substituents are as defined in claim 1.
